(19) United States
(12) Patent Application Publication
(10) Pub. No.: US 2026/0185109 A1
(43) Pub. Date: Jul. 2, 2026

(54) SYSTEMS AND METHODS FOR QUANTIFICATION AND MANIPULATION OF GENOME GEOMETRY FOR CELLULAR REPROGRAMMING AND COMPUTATION

(71) Applicants: Northwestern University, Evanston, IL (US); Ann and Robert H. Lurie Children's Hospital of Chicago, Chicago, IL (US)

(72) Inventors: Vadim Backman, Evanston, IL (US); Luay M. Almassalha, Evanston, IL (US); Igal G. Szleifer, Evanston, IL (US); Marcelo A. Carignano, Evanston, IL (US); Kyle Lauchlin MacQuarrie, Chicago, IL (US)

(21) Appl. No.: 19/438,416

(22) Filed: Dec. 31, 2025

Related U.S. Application Data
(60) Provisional application No. 63/740,751, filed on Dec. 31, 2024.

(51) Int. Cl. C12N 15/67; C12N 9/10; C12N 9/12

(52) U.S. Cl. CPC C12N 15/67; C12N 9/1007; C12N 9/1241; C12N 2830/46

(57) ABSTRACT

Disclosed are compositions, systems, and methods of regulating gene transcription in a cell that include forming, stabilizing, and preventing decay of chromatin packing domains in the cell. Methods of forming chromatin packing domains include forming nascent chromatin packing domains that transition to mature packing domains.

### CROSS REFERENCE TO RELATED APPLICATIONS

This application claims priority to U.S. Provisional Application No. 63/740,751, filed Dec. 31, 2024. The entire contents of which are incorporated herein by reference.

### GOVERNMENT RIGHTS

This invention was made with government support under grant numbers CA268084, CA228272, CA261694 awarded by The National Institutes of Health and grant number EFMA-1830961 awarded by the National Science Foundation. The government has certain rights in the invention.

### BACKGROUND

Even as the tools to probe genome structure and function have rapidly advanced, the conceptual framework around structure-function has converged on the dichotomy between open (transcriptionally active, low-density, euchromatin rich, A-compartment) and closed (transcriptionally repressed, high-density, heterochromatin rich, B-compartment) states¹⁻⁸. There are several prominent models centered around this framework: loop extrusion as a barrier element of epigenetic spreading^{9, 10}, self-attraction of nucleosome markers producing segregation¹¹⁻¹⁴, hierarchical functional assemblies^{5, 15, 16}. The central goal of all these models is explaining how the partitioning of the 2-meters long human genome into nuclei several-micron in diameter produces structures that can effectively regulate the nuclear processes relevant for cell function: transcription, replication, and DNA repair.

At the level of transcription, these models all face a formidable challenge: dichotomizing the genome into 2 distinct groups simply does not account for the patterns in expression that are observed when chromatin is induced to transform from one state to the other^{6, 7, 17-19}. Specifically, inhibiting heterochromatin enzymes does not result purely in transcriptional activation¹⁷⁻¹⁹. Related to this paradox are the complexities observed in compartments and sub compartments in high-throughput conformation capture (Hi-C). Ever since the early descriptions, compartments defied a pure delineation into active and inactive nucleosome post-translational modifications^{6, 7}. For example, as observed in Rao et al⁷, repressive histone markers such as H3K9me3 can be as strongly correlated with transcriptionally active A2 sub-compartments as transcriptionally active markers such as H3K4me3⁷. Likewise, the correlation of H3K9me3 is comparable to those of euchromatin marks in B sub-compartments⁷. Analogous to these limitations, gene expression cannot be predicted solely on the basis of the combinatorial presence of histone marks or accessibility at a gene loci²⁰⁻²². These limitations become more pronounced when considering that inhibition of loop extrusion by RAD21 depletion has muted impacts on gene transcription even as H3K9me3 increases as measured by chromatin immunoprecipitation sequencing (ChIP-Seq)²³. Compounding this problem, the loss of RAD21 has no discernable impact on the amount of heterochromatin within spatially resolved chromatin aggregates observed on super resolution imaging²⁴. Collectively, these findings demonstrate that ensemble connectivity features alone may not translate into space-filling conformations due to potential differences between genome topology and chromatin conformation. Whereas topological properties such as topologically associated domains (TADs) are ensemble properties of thousands of cells^{6, 7}, chromatin conformation describing the chromatin polymer at the level of an individual cell is not merely the observed connections. Therefore, integrating the concepts of transcription, accessibility, nucleosome modification, and loop extrusion into a cohesive system remains elusive.

### SUMMARY

An aspect of the disclosure is a method of regulating gene transcription in a cell by forming one or more chromatin packing domain in the cell, stabilizing the one or more chromatin packing domain in the cell, and preventing decay of the stabilized chromatin packing domain. Forming a chromatin packing domain includes forming a nascent chromatin packing domain, stabilizing the nascent chromatin packing domain, and preventing decay of the chromatin packing domain.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F. Packing domains are the predominant supra-nucleosome structure independent of the cell line. (FIG. 1A) High-resolution mean projection from ChromSTEM in A549, BJ, and HCT-116 cells with a representative domain tomogram from A549 cell. Scale bars 200 nm, domain size 200×200 nm. FIGS. 1B-1E Analysis of structural properties of domains for these distinct cell types demonstrates heterogeneity of domain structures by cell type. (FIG. 1B) Scaling of chromatin packing ranges between 2-3 in all cell types. (FIG. 1C) Domain radius typically range from 50-200 nm between all cell lines (FIG. 1D) Variations in chromatin volume concentration within domains is observed. (FIG. 1E) Quantification of chromatin packing efficiency. (FIG. 1F) Representative spatial distribution of density from domain interiors toward their periphery demonstrates a conserved, power-law geometry with a decay to the average nuclear density at the periphery. Domain boundary for blue (68 nm), black (86 nm), and green (72 nm).

FIGS. 2A-2E. Stochastic returns and excluded volume influence domain interactions with remodeling enzymes. (FIG. 2A) Schematic representation of modeling frameworks in chromatin. A random walk and a confined random walk are cases of nucleosomes with fixed distances. In both cases, the produced structure results in a limiting case of chromatin domains with a D=2 (random walk) and D=3 (confined random walk, or fractal globule). Forced attractions can produce more complex structures, but the discrete partitions results in two separated structures (low density—A, high density—B states). Stochastically forced returns that depend on the distance between the nucleosomes produces corrugated, mass-fractal structures that resemble ChromSTEM resolved domains. These have a continuous decrease in density from high density cores (red) to intermediate conditions (yellow) and finally to outer zones (blue) prior to transition to interdomain space. (FIG. 2B) Representative chromosome fragment from SR-EV demonstrating the formation of chromatin packing domains due to the intersection of stochastic return events and the excluded volume of monomers (nucleosomes). (FIG. 2C) The molecular mass in kilodaltons verses the radius of gyration, Rg, in nanometers predicted from AlphaFold configurations organized by enzyme function (red-heterochromatin, yellow-transcription factors, blue-euchromatin). Euchromatin enzymes have a radius that is approximately twice the size of heterochromatin enzymes, p-val<0.001. (FIG. 2D) Simulated protein penetration relative to the average penetration as a function of size demonstrating preferential localization of larger enzymes to low density regions (CVC<0.1) and small molecules minimally impacted by higher CVC. (Black—1 nm radius, small molecule. Red—3 nm radius, heterochromatin protein. Yellow—4.5 nm radius, transcription factors. Blue—6 nm radius, euchromatin protein). (FIG. 2E) Molecule size results in differential localization as a function of domain CVC in SR-EV configurations with increased relative concentration of smaller molecules to domain interiors (3 nm heterochromatin vs 6 nm euchromatin enzyme shown).

FIGS. 3A-3D. A phenomenological model of domain self-assembly. (FIG. 3A) Visual schematic of domain structures within the nucleus demonstrating their intersection with RNA polymerase, cohesion, and nucleosome modifiers. Nascent domains and mature domains represent temporally evolving processes due to the intersection of nucleosome remodeling with return/loop mediating processes. Proposed 3-rule framework for domain assembly, stabilization, and function. Rule 1) The process of returns creates local density variations resulting in nascent domain formation. Rule 2) The excluded volume properties of domains and nucleosome remodeling enzymes results in preferential localization of heterochromatin remodeling enzymes to the interior of domains. Rule 3) Transcription depends non-monotonically on local crowding and requires optimal zone configurations to accelerate. (FIG. 3B) Model predictions of the effect of transcriptional activation on the total number of observed loops (blue), entropic loops (green), and polymerase-mediated loops (purple) over time after transcriptional initiation. The negative frequency of entropic loops denotes the loss of entropically mediated loops over time as transcriptionally mediated loops and total loops increase. (FIG. 3C) Model predictions of the change in chromatin volume concentration overtime following initiation of transcriptional reactions at 1 hour with resulting accumulation of heterochromatin within the domain interior. Inhibition of transcription results in the converse phenotype with the decrease in density and loss of heterochromatin formation. (FIG. 3D) Consequence of transcriptional activity on polymer scaling, D, within domains after initiation at 1 hour demonstrating the maturation of domain structures with transcriptional activation (D 2.2→2.8).

FIGS. 4A-4G. Nascent domains are formed from transcriptionally mediate or cohesin-generated returns. (FIGS. 4A-C) Analysis of native loop domains upon depletion of RAD21 (FIG. 4A), depletion of RNA polymerase II (FIG. 4B), and transcription inhibition with 4 mm ActD (FIG. 4C) demonstrating the loss of loop anchors with these perturbations. RAD21 and Pol-II depletion was achieved by 5-Ph-IAA treatment over 6 hours. (FIGS. 4D-E) Representative packing domains (200 nm×200 nm) from RAD21 depleted cells (FIG. 4D) and 4 mm ActD treated cells FIG. 4 (E) showing porous structure and high-density cores are maintained in mature domains. Cross-sectional analysis of domains by size and packing efficiency demonstrates a disproportionate loss of low efficiency, small domains with inhibition of transcription and RAD21 depletion. (FIG. 4F) Live-cell PWS nanoscopy in RAD21 depleted HCT-116 cells at 4 hours demonstrating no impact on average chromatin scaling, D and a decrease in fractional moving mass, consistent with impaired domain formation but retention of overall higher-order structure. (FIG. 4G) Live-cell PWS nanoscopy of ActD treated BJ fibroblast cells demonstrating a decrease in D and decrease FMM consistent with the increase in decaying domains (large, low packing efficiency) and impaired domain formation.

FIGS. 5A-5I. Domains spatially couple heterochromatin, euchromatin, and active RNA polymerase. (FIG. 5A) Multiplex SMLM demonstrating the spatial localization of heterochromatin (H3K9me3, magenta), euchromatin (H3k27ac, yellow), and active RNA polymerase II (Serine 2 phosphorylated, Pol2-PS2, blue). This shows the complex spatial organization of chromatin into unified domain structures with heterochromatin cores (red) supporting Pol2-PS2 within an ideal domain functional zone (gray). (FIGS. 5B-D) Multiplexed SMLM demonstrating the impact of inhibition of EZH2 (GSK343), HDACs (TSA), and transcription (ActD) on domain structure. Although mature domain structures remain, the disruption of transcription results in loss of H3K9me3 cores and their dissociation from active RNA polymerase. TSA mediated HDAC inhibition results a loss of heterochromatin and euchromatin marks with the most pronounced decrease observed in the nuclear interior. (FIG. 5E) Quantification of H3K9me3 core size upon HDAC inhibition, EZH2 inhibition and transcriptional inhibition demonstrating a decrease in core size in all conditions imaged in Hela cells above. (FIG. 5F) Quantification of Pol2-PS2 distribution upon HDAC inhibition, EZH2 inhibition and transcriptional inhibition demonstrating a decrease in total Pol2-PS2 upon disruption of heterochromatin formation in Hela cells above. (FIG. 5G) Quantification of the frequency of Pol2-PS2 observed near a surrounding H3K9me3 core in the above conditions. At baseline ˜60% of Pol2-PS2 in HeLa cells is spatially associated with domains and this is lost both with HDAC inhibition and disruption of transcription by ActD. (FIG. 5H) Analysis from ChIP-Seq of the correlation between chromosome wide (a global measure) of heterochromatin markers with Pol2 serine-5 phosphorylated (Pol2-Ps5) isoform (initiated transcription). (FIG. 5I) Linear proximity analysis of constitutive heterochromatin (H3K9me3) and euchromatin (H3K4me3) as a function of Pol2-Ps5 density on gene bodies. Findings are consistent with H3K27me3 associating with nascent domains and H3K9me3 with mature domains. In contrast, H3K4me3 distance increases as a function of Pol2-Ps5 density likely due localization in the outer zone.

FIGS. 6A-6H. Divalent ion chelation results in domain collapse. (FIG. 6A) Nuclear volume increases with chelation via BAPTA (Calcium) and APDAP (Magnesium) within 1 hour. (FIG. 6B) Representative multiplexed SMLM with Pol2-PS2 (blue) and H3K9me3 (magenta) in HCT-116 cells with and without chelation of divalent ions (calcium, magnesium) via BAPTA treatment at 1 hour. (FIG. 6C) Quantification of divalent chelation on H3K9me3 domains demonstrating a global loss of domain interiors. (FIGS. 6D-6E) Quantification of Pol2-PS2 demonstrating an increase in dissociation upon chelation. (FIGS. 6F-6H) Live-cell PWS nanoscopy of BAPTA treated HCT-116 cells at one hour demonstrating a decrease in D (FIG. 6G) and decrease FMM (FIG. 6H) consistent with domain collapse and inhibition of domain formation.

FIGS. 7A-7H. Inhibition of heterochromatin enzymes paradoxically suppresses transcription in situ due to impairment of ideal conditions. (FIGS. 7A-7F) Representative multiplexed SMLM of nascent RNA measured by EU synthesis (blue) and H3K9me3 (magenta) in HCT-116 in controls compared to inhibition of EZH2 (FIG. 7B) and HDACs (FIG. 7C) demonstrating the profound loss of synthesis with inhibition of heterochromatin enzymes. (FIGS. 7G-7H) Quantification of the effect of heterochromatin enzyme suppression on RNA synthesis throughout the nucleus in comparison to adjacent to the nuclear border demonstrating loss of transcription independent of the nuclear region in HCT-116 cells. Note that the average CVC in HCT-116 cells is ˜0.2-0.35 on ChromSTEM above, indicating that at baseline these cells are near physiochemical conditions at baseline. HDAC inhibition and EZH2 inhibition can still increase local transcription for initially high density regions and globally in cell lines with a higher initial CVC (>0.35).

FIGS. 8A-8K. Domain assembly occurs during myogenic differentiation and depends on chromatin volume concentration. (FIGS. 8A-8C) Transformation of the myogenic regulator, Myog, chromatin loci during development demonstrating the loss of loops (Rule 1) during development with accompanying increase in heterochromatin adjacent to the gene body (Rule 2) and acceleration of transcription (Rule 3). (FIGS. 8D-8F) Transformation of the fast-twitch myosin heavy chains (Myh 1-2) chromatin loci during myoblast differentiation with loss of adjacent loop (Rule 1) with accompanying increase in heterochromatin adjacent to the gene body (Rule 2) and amplified transcription (Rule 3) of structural myogenic proteins. (FIG. 8G) Representative configuration from SR-EV of chromosome 17 (scale bar 200 nm) with color coding representing the coordination number (CN) representing the number of nucleosomes in contact. A CN of less than 5 represents a outer zone-density configuration, 6-7 optimal transcriptional configuration, and greater than 7 representing an interior configuration. (FIG. 8H) Generated configuration from SR-EV of Myh1 representing quantified CN of exon segments with introns color coded in yellow (scale bar 40 nm). Remarkably, exon segments are frequently in outer zone or ideal configurations. (FIG. 8I) Configuration from (FIG. 8H) with quantified CN of intron segments with exons color coded in green (scale bar 40 nm). Inverse to exons, intronic elements are frequently found in domain interior configurations. (FIG. 8J) Average coordination number as a function of CVC with lower densities shifting toward outer zone configurations. (FIG. 8K) Average coordination number of Myh1 exons per nucleosome as a function of the exon segment at CVC of 0.16 consistent with localization of coding elements into ideal transcriptional densities depending highly on CVC.

FIG. 9. Packing domains are power-law structures. Log-log plot of mass verses domain radius demonstrating power-law scaling between 10 nm and 74 nm in A549 cells. The corresponding D value calculated is 2.54.

FIGS. 10A-10F. Analysis of chromatin loop domains with transcriptional inhibition and depletion of RAD21. (FIGS. 10A-10C) Representative insulation plots from control cells compared to RAD21 depleted, RNA Polymerase II depleted, and treated with actinomycin D demonstrating loss of loop strength globally. (FIGS. 10D-10E) Analysis of loop size and distribution demonstrating an increase in large, weak loops upon transcriptional inhibition. (FIG. 10F) Domain analysis from ChromSTEM tomogram in control fibroblast compared to Actinomycin D treated fibroblasts demonstrating loss of nascent domains and an increase in decaying domains.

FIG. 11. Distance analysis from heterochromatin domain boundary for serine-2 phosphorylated RNA polymerase and H3K27ac. Heterochromatin domain cores were identified on multiplexed single molecule localization microscopy using DBScan. Localizations of active RNA polymerase Pol-II PS2 and euchromatin were then calculated relative to the distance from the domain boundary in nm. Shaded area represents 75% percent of the localized events with full distribution shown. Both features localize adjacent to domain boundaries within 2-5× the radius of the observed domain core.

FIGS. 12A-12C. Spatial analysis of (FIG. 12A) H3K9me3, (FIG. 12B) H3K27AC, and (FIG. 12C) PolII-PS2 in multiplexed single molecule localization microscopy. Analysis of chromatin marks and active polymerase in DMSO controls, GSK343 treated, and TSA treated cells showing the central loss of euchromatin marks with inhibition of heterochromatin enzymes. The central loss of heterochromatin markers is accompanied by the loss of euchromatin marks consistent with domain coupling.

FIGS. 13A-13B. Live cell PWS nanoscopic imaging of cells treated with GSK343 and TSA compared to controls. (FIG. 13A) On inhibition of EZH2 (GSK343 treatment), there is a decrease in D and a decreased in FMM consistent with the impairment of domain maturation processes. (FIG. 13B) On HDAC inhibition (TSA treatment) there is a larger decrease in D and FMM consistent with the disruption of mature domains and domain formation in live cells.

FIG. 14. Analysis of myosin heavy chain loci in myoblasts. Myh1 and Myh2 are the predominant skeletal muscle myosin heavy chains with an absence of H3K9me3, H3K4me3, and low-levels of expression of these genes in immature myoblasts.

FIG. 15. Analysis of myosin heavy chain loci in myotubes. Analysis of Myh1 and Myh2 in myotubes (mature muscle cells) showing an accumulation of H3K9me3 and H3K4me3 with amplification of gene transcription. Note that heterochromatin foci predominantly form at non-coding segments of within or adjacent to these genes.

FIG. 16. Table listing respective enzymes, size, mass, the location of the PDB structure and name. Accompanying estimated radius of gyration (nm) compared to the mass in Daltons. Abbreviations: C—Class, EU—Euchromatin, HC—Heterochromatin, TF—Transcription Factor P—Polymerase.

FIG. 17. Table listing ENCODE data for each experiment file, method, source, marker, and type.

FIGS. 18A-18F. (FIG. 18A) Synthesized RNA (gray, bottom) on exons is concurrently associated with heterochromatin core (H3K9me3) deposition within non-coding regions within genes (introns) and adjacent bodies (intergenic space) across multiple cell/tissue types from each germ layer. (FIG. 18B) Translation of linear genetic information into a packing domain for transcription to efficiently occur on a ‘ideal zone surface’. The linear components are assembled on the surface of the volume, generating a nearly linear path for the polymerase along the reaction surface. (FIG. 18C) Comparison of mass-fractal domain organization with unconstrained loops. A 100 Kbp loop assembly would span nearly the radius of the nucleus. (FIGS. 18D-18E) Limiting cases of mass fractals with D=2 and D=3, which have uniform density distributions. Reactions in the limiting case of D=3 would have to occur at the domain boundary instead of an ideal reaction zone. (FIG. 18F) Simplified model of conformational self-assembly of packing domains. Sustained transcription generates a nascent domain which mature from the combination of divalent ions and heterochromatin remodeling enzymes positioning along the density gradient to generate maturation. The mature domain produces an ideal reaction zone for sustained RNA synthesis.

FIGS. 19A-19G. (FIG. 19A) Comparison of RYR1 and MYH1 gene bodies at scale. The proportion of RYR1 composed of introns is not linearly proportional to the exon content. (FIG. 19B) Histogram of protein coding genes in the human genome showing that the median fraction is ˜10% coding with a subset of genes that are almost completely exon. (FIG. 19C) Plot of the ratio of exon (surface)/intron (volume) compared to the gene length of human genes. The constant, m, reflects the information density with n set to 1. (FIG. 19D) Randomization of exon/intron segments results in the statistically grounded null hypothesis of no relationship between length and exon/intron ratios with a fraction approaching the median of 0.1. (FIG. 19E) Intron length is a power-law of exon length for protein coding genes with values of m and γ as reported. (FIG. 19F) Schematic representation of gene composition in relation to m and γ, indicating that high γ indicates more non-coding volumetric elements are present within a segment. (FIG. 19G) Relationship between γ and D depends on the proportion of the exons making up the surface where β=1 indicates the entire exon contents are on a hard surface.

FIGS. 20A-20I. (FIGS. 20A-20B) Analysis of genes differentially expressed in human organs with each location chosen to represent a different germ-line origin (esophagus-endoderm, muscle—mesoderm, cerebellum—ectoderm). Genes involved in end-organ function are power-law assemblies. (FIGS. 20C-20D) Analysis of Yamanaka factors (YFs+) in comparison to HOX genes and other transcription factors. YFs+ and HOX genes are generally deviations from power-law assemblies favoring exon density whereas most transcription factors in humans are power-law. (FIGS. 20E-20I) Comparative analysis of gene composition as a function of body plan complexity. There is a gradual transition from exon-rich (E/I>1) from S. cerevisiae into 1:1 distribution in C. elegans that then transition to primarily power-law compositions as complexity increased.

FIGS. 21A-21D. (FIGS. 21A-B) Analysis of non-protein coding transcribed elements including miRNA, lncRNA, pseudogenes, etc within the human genome displaying comparable assembly properties to human protein coding genes. (FIGS. 21C-D) Subgroup analysis of HOX-associated (HOX-As) long non-coding RNA including (HOTAIR, HOTTIP etc) compared to other lncRNA such as (BDNF-AS, PVT1, TSIX, XIST, MALATI etc.). Overall, frequently transcribed but non-protein coding genes organize in a similar fashion to their protein coding counterparts.

FIGS. 22A-22E. (FIG. 22A) Schematic representation of the organization of two genes on a chromosome folded into two separate domains separated by a linker or hinge element. (FIG. 22B) Analysis of the structure of chromosomes 1-6 in the positive strand orientation showing power-law assemblies of noncoding junk (volumetric) elements scaling as a power-law of coding (ideal zone surface) elements. (FIG. 22C) Random repositioning of a coding segment with it's adjacent unit of junk results in the generation of SA/V elements whereas the random repositioning of exons alone generates very large clusters. These findings are consistent with the functional coupling of an exon-junk unit as the minimal geometric unit. (FIGS. 22D-E) Analysis of chromosome SA/V compositions considering only protein coding (FIG. 22D) or non-protein coding (FIG. 22E) positions. Overall, genes remain grouped as SA/V compositions. All analysis of composition was performed with hinge elements set at 200 bp or smaller for FIG. 22B-D in the positive read orientation. Similar findings were observed for all chromosomes in both orientations.

FIGS. 23A-23G. Comparative analysis of chromosome geometry across organism complexity in eukaryotic species. S. cerevisiae organizes into linear compositions (Junk ˜1.5*Exon) length whereas organisms with complex organs primarily generate SA/V configurations. Notably, C. elegans and D. melanogaster contain a mix of linear assemblies as well as SA/V with a marked transition occurring in D. rerio onward. For simplicity of comparison across genomes, γ was held constant at 2.2 and only m was varied. For simplicity due to the variable number of chromosomes in each species, only the first 6 representative chromosomes from each species are shown, all of the results were verified to hold for all chromosomes for that species in both orientations.

FIGS. 24A-24J. (FIGS. 24A-C) Analysis of the composition of loops and topologically associated domains of chromosome 16 in HCT-116 cells on ChiaPET (FIG. 24A) and on Micro-C (FIGS. 24B, 24C). Overall, the majority of loops generated by either Polr2a or CTCF have a constant amount of volumetric DNA relative to the coding proportion. Likewise, TADs and Micro-C defined loops have similar composition in either strand orientation. The constant proportion of volumetric DNA to exon would suggest that these represent features along a surface. (FIGS. 24D-G) Analysis of the overlap in position between an observed hinge (<200 bp) with enhancers (D), H3K27ac (FIG. 24E), active RNA polymerase II (FIG. 24F) and CTCF (FIG. 24G) compared to hinge elements generated by randomly repositioning exons in all chromosomes in HCT-116 cells. Statistical testing for all comparisons was performed and the p-value was less than 0.01. (FIG. 24H) MYH1 locus in myoblasts (top) compared to differentiated myotubes (bottom) demonstrating loss of two DNAse peaks in non-coding segments (arrows) with concordant increase in H3K9me3 in intergenic regions. DNAse peaks were scaled between 0-3, all other peaks between 0-5. (FIG. 24I) Analysis of the distance between the median distance of active RNA-polymerase II and the nearest H3K9me3 in induced pluripotent GM23338 cells demonstrating decreased distance with increased amount of active Pol II on the gene body. R2=0.95 for H3K9me3 and 0.99 for H3K4me3 vs Pol II Ps5. (FIG. 24J) Analysis of the per-chromosomal density of H3K27me3 and H3K4me3 within the esophageal mucosa of 4 patients. The amount of heterochromatin within chromosomes correlates with the degree of euchromatin across multiple human samples R²=0.749.

FIGS. 25A-25D. Steric exclusion of antibodies results in non-linear association between nucleosome modification and DNA density in situ. (FIG. 25A) Two color single-molecule localization microscopy of EdU and BrdU staining of DNA. EdU staining utilizing Click-it chemistry with a small molecule probe (˜2 nm) results in differential localization compared to antibody staining of DNA with BrdU (antibody ˜10 nm). Inset demonstrates staining behavior of antibody (purple) around the outside of a high-density domain (yellow) with comparable emission events to regions devoid of DNA (green) as identified based on EdU penetration. (FIGS. 25B-25C) Two color single-molecule localization microscopy of EdU compared to H3K9me3 (FIG. 25B) and H3K27me3 (FIG. 25C) (both high-density heterochromatin) demonstrating localization in situ depends on size exclusion principles. H3K9me3 and H3K27me3 staining using antibody labeling results in accumulation near domain interior. (FIG. 25D) Two color single-molecule localization microscopy of EdU and H3K4me3 (low density eu-chromatin) showing colocalization as a function of DNA density.

FIGS. 26A-26B. Measurement of chromosomal size in myogenesis. (FIG. 26A) Observed radius of chromosome 2 in immature muscle cells (myoblasts, MB) compared to mature muscle cells (myotubes, MT) demonstrating a radius of ˜1-2 microns in size for Chromosome 2 in mature muscle cells. (FIG. 26B) Observed radius of chromosome 18 in immature muscle cells (myoblasts, MB) compared to mature muscle cells (myotubes, MT) demonstrating a radius of ˜500 nm-1.3 microns in size for chromosome 18 in mature muscle cells. Even accounting for domain swelling due to FISH microscopy preparation, the occupied area for each chromosome would be comparable to a fully extended enhancer-promoter loop of 100 kbp despite orders of magnitude differences in composition.

FIGS. 27A-27D. Variant Isoform analysis and non-coding gene behavior. (FIG. 27A) Analysis of behavior of all protein coding genes demonstrating inverse relationship between surface area to volume ratio verses gene length across all isoforms. (FIG. 27B) Analysis of behavior of non-protein coding genes (long non-coding RNA, pseudogenes, etc) demonstrating inverse relationship between surface area to volume ratio verses gene length independent of transcript products. (FIGS. 27C-D) Subselected long-noncoding RNA sequences (SI Table 3 for selected genes) demonstrates similar power-law behavior as that observed in protein coding genes independent of the RNA product function.

### DETAILED DESCRIPTION

The present disclosure provides compositions, systems, and methods for use in regulating genome structure and function, including regulating gene transcription and chromatin structure in a cell. There is broad consensus that the smallest functional physical structure of chromatin is nucleosomes organized as “beads on a string” or clutches (5-25 nm)²⁵. Above this scale, however, there is limited consensus. Using a variety of methods and contrast agents that include but are not limited to chromatin electron microscopy (ChromEM)²⁵, structured illumination (SIM)^{24, 26, 27}, single molecule photon localization microscopy (SMLM) (28), live cell spectroscopic nanoscopy²⁹, and DNA-paint^{26, 30-32}, supra-nucleosome organization has been described as variably sized structures such as nucleosome clutches comprising a small number of nucleosomes³³, TAD-like domains (200-500 nm)^{30, 31}, nanodomains (100-200 nm)²⁶, chromatin fibers (50-200 nm)³⁴, chromatin domains (100-200 nm)^{24, 35}, and packing domains (50-200 nm)²⁹. “Chromatin packing domain” or “packing domain” refers to a region within a cell nucleus where chromatin is condensed or densely packed and organized.

In this disclosure, an intriguing hypothesis was tested to resolve the paradoxical behavior of transcription upon heterochromatin enzyme inhibition while linking the various structures into a cohesive framework. Specifically, it was expected that the observed physical properties of ChromEM resolved packing domains reflects their structure-function life-cycle with respect to gene transcription: 1) small, low density (nascent) domains are formed by processes such as cohesion-mediated loops and RNA polymerase mediated promoter-promoter/promoter-enhancer interactions; 2) the size of nucleosome remodeling enzymes results in preferential penetration of heterochromatin enzymes in areas of high density within domain cores to mature the structure; and 3) the produced density near domain boundaries provides an improved physical scaffold for RNA synthesis by stabilizing the binding of the polymerase and transcription factors within intermediate densities. The result is that chromatin isn't organized into two distinct groups, but into a unified, dynamic domain forming system.

Collectively, this disclosure demonstrates that: 1) chromatin in vitro assembles into packing domains independent of the cell line; 2) packing domains are heterogeneous with a broad distribution of sizes, density, and packing efficiencies that reflect their function; 3) the act of gene transcription and cohesin-mediated loop extrusion facilitates the formation of nascent packing domains but this is insufficient for maturation to occur; 4) transcription, enzymatic size, density, and divalent ionic concentrations mechanistically maintain domain stability; 5) packing domains do not appear to be the physical manifestation of topologically associated domains (TADs); and 6) Improved gene transcription depends on domain stability. How the disruption of domain self-assembly from physical considerations can have physiologic consequences are shown by modeling how nuclear swelling would disrupt myogenic domains with a focus on sarcopenia (deleterious loss of muscle mass). Given that sarcopenia is independently associated with all-cause adult morality and quality of life, understanding domain transformation can provide a mechanistic link between physical genomic organization, gene transcription, and human disease.

An aspect of the disclosure is methods of regulating gene transcription in a cell by forming one or more chromatin packing domain in the cell, stabilizing the one or more chromatin packing domain in the cell, and preventing decay of the one or more chromatin packing domain. Forming the one or more chromatin packing domain includes forming a nascent chromatin packing domain. A nascent chromatin packing domain may be formed by one or more process including transient contact of chromatin fiber from spatial confinement, loop extrusion, and transcriptionally mediated contact of chromatin fiber.

A chromatin packing domain may be stabilized by maintaining or increasing nucleosome-modifying enzymes in the cell. Exemplary nucleosome-modifying enzymes include heterochromatin enzyme, euchromatin enzyme, polymerase, and cohesin. A chromatin packing domain may also be stabilized by maintaining ion concentration in the domain. Ions in packing domains include calcium and magnesium. In embodiments, calcium and/or magnesium ion are in the domain. Preferably, the ions are at concentrations that stabilize the domain.

In aspects of the disclosure, a stabilized chromatin packing domain contains zones that function in regulating gene transcription. The zones include a high-density inner core zone, an intermediate zone, and low-density outer zone. A high-density inner core zone may include heterochromatin enzyme and/or euchromatin enzyme. In embodiments, heterochromatin enzyme may be the predominant enzyme in a high-density inner core. An intermediate zone may include heterochromatin enzyme and/or RNA polymerase enzyme. A low-density outer zone may include a euchromatin enzyme. A euchromatin enzyme may be the predominant enzyme in a low-density outer zone.

Further, the disclosure demonstrates that exons are geometrically coupled to their adjacent non-transcribed “junk” (introns/intergenic DNA) segments to form power-law surface area-to-volume (SA/V) assemblies oriented with the transcriptional reading frame reflecting the structure of mature packing domains. The inventors show a distinct transition from a linear to a power-law genome in complex, multicellular organisms. Collectively, this indicates a physically-encoded “geometric code” linking transcribed and adjacent non-transcribed units into cohesive physical structures such as packing domains.

In embodiments, forming one or more chromatin packing domain may include modulating a surface-to-volume assembly of chromatin segments. In some embodiments, modulating a surface-to-volume assembly comprises pairing exons with an adjacent volumetric DNA segment. As used in the disclosure, volumetric DNA segments include introns and/or intergenic segments.

As used throughout this disclosure, “junk”, “junk DNA”, and “junk segments” may be used interchangeably to refer to both introns and intergenic segments of DNA and to demarcate DNA not generally transcribed into an RNA product. As disclosed, geometric positioning of non-transcribed junk DNA with exons to produce SA/V assemblies observed as packing domains redefines ‘junk’ into ‘volumetric’ DNA. The redefined volumetric DNA shows a crucial function for the introns/intergenic DNA to support the transcription of complex genes, provide physical durability, and act as a non-mutational system for genomic sampling by optimizing the complexity of genomic space.

The present disclosure is not limited to the specific details of construction, arrangement of components, or method steps set forth herein. The disclosed compositions, systems, and methods are capable of being made, practiced, used, carried out and/or formed in various ways that will be apparent to one of skill in the art in light of the disclosure. The phraseology and terminology used throughout the disclosure is for the purpose of description only and should not be regarded as limiting to the scope of the claims.

The terms “a,” “an,” “the” and similar references in this disclosure, including in the context of the claims, include both the singular and the plural, unless indicated otherwise or the context clearly indicates otherwise. Ordinal indicators, such as first, second, and third, as used in the description and the claims to refer to various structures or method steps, are not meant to be construed to indicate any specific structures or steps, or any particular order or configuration to such structures or steps. All disclosed methods can be performed in any suitable order unless otherwise indicated or otherwise clearly contradicted by context. The use of any and all examples, or exemplary language (e.g., “such as”) is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

The use in this disclosure of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values in the disclosure are merely intended to serve as a shorthand method of referring individually to each separate value falling within the range, unless otherwise indicated, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited in this disclosure are fully incorporated by reference, unless explicitly indicated otherwise. The present disclosure shall control in the event there are any disparities between any definitions and/or description found in the cited references.

### EXAMPLES

The invention can be further understood in view of the following non-limiting examples. The following examples are meant only to be illustrative and are not meant as limitations on the scope of the disclosure, invention or of the appended claims.

### Example 1

In single cells, variably sized nanoscale chromatin structures are observed but it is unknown if these form a cohesive framework that regulates RNA transcription. This disclosure demonstrates that the human genome is an emergent, self-assembling, reinforcement learning system. Conformationally-defined heterogenous, nanoscopic packing domains form by the interplay of transcription, nucleosome remodeling, and loop extrusion. As shown here, packing domains are not topologically associated domains. Instead, packing domains exist across a structure-function life-cycle that couples heterochromatin and transcription in situ, explaining how heterochromatin enzyme inhibition can produce a paradoxical decrease in transcription by destabilizing domain cores. Applied to development and aging, the pairing of heterochromatin and transcription at myogenic genes that could be disrupted by nuclear swelling is shown. In sum, packing domains represent a foundation to explore the interactions of chromatin and transcription at the single cell level in human health.

### Materials and Methods

### Cell Culture

HCT116 cells (ATCC, #CCL-247) and U2OS cells (ATCC, #HTB-96) were grown in McCoy's 5A Modified Medium (#16600-082, Thermo Fisher Scientific, Waltham, MA). HeLa cells (ATCC, #CCL-2) were cultured in RPMI 1640 medium (Thermo Fisher Scientific, Waltham, MA; #11875127). BJ cells were cultured in Minimum Essential Media (ThermoFisher Scientific, Waltham, MA, #11095080). All cell culture media was supplemented with 10% FBS (#16000-044, Thermo Fisher Scientific, Waltham, MA) and penicillin-streptomycin (100 μg/ml; #15140-122, Thermo Fisher Scientific, Waltham, MA). All cells were maintained under recommended conditions at 37° C. and 5% CO2. Cells were allowed at least 24 hours to re-adhere and recover from trypsin-induced detachment. All imaging was performed when the surface confluence of the dish was between 40-70%. All cells in this study were maintained between passage 5 and 20. All cells have been tested for mycoplasma contamination (ATCC, #30-1012K) before starting experiments, and they have given negative results.

### Drug Treatments

AID2 cell lines: HCT116 RAD21-mAID-Clover CMV-OsTIR1(F74G) cells(58, 59) were plated at 50,000 cells per well of a 6-well plate (Cellvis, P12-1.5H-N). To induce rapid and efficient depletion of mAID-fused proteins (RAD21), 5-Ph-IAA (#HY-134653, MedChemExpress), 5-(3,4-dimethylphenyl)-indole-3-acetic acid, 5-(3-methylphenyl)-indole-3-acetic acid and 5-(3-chlorophenyl)-indole-3-acetic acid was dissolved in DMSO to make a 500 mM stock solution, and further diluted with DMSO to working stock solution of 1 mM immediately before the experiment. A final concentration of 1 μM of 5-Ph-IAA was added to HCT116 RAD21-mAID-Clover CMV-OsTIR1 (F74G) cells for 6 hours.

Inhibition of transcription: HCT116 and BJ cells were plated at 50,000 cells either per well of a 6-well plate (Cellvis, P12-1.5H-N). For inhibition of RNA synthesis, actinomycin D(60) (#11805017, Gibco) was dissolved in DMSO to make an 8 mM stock solution, and further diluted with cell media to make a working solution of 80 μM immediately before the experiment. A final concentration of 4 μM was added to HCT116 and BJ cells for 1 hour to completely arrest all polymerases.

Inhibition of EZH2 and HDACs: For EZH2 inhibition, GSK343 (#1346704-33-3, Millipore Sigma) was dissolved in DMSO to make a 10 mM stock solution and added to U2OS cells at a final concentration of 10 μM for 24 hours. For histone deacetylase (HDAC) class 1 and 2 inhibition, Trichostatin A (TSA)(3, 18) (#58880-19-6, Millipore Sigma) was added to HCT116 cells at a final concentration of 300 nM for 24 hours.

Chelation of Divalent Cations: For ionic inhibition, membrane-permeable 1,2-bis(2-aminophenoxy) ethane-N,N,N′,N′-tetraacetic acid tetrakis(acetoxymethyl ester) (BAPTA-AM)(87) (#B6769, Invitrogen) was dissolved in DMSO to make a 10 mM stock solution and added to HCT116 cells at a final concentration of 10 uM for 1 hour to chelate calcium and magnesium ions.

### Live Cell Partial Wave Spectroscopic (PWS) Microscopy Acquisition and Analysis

For live-cell measurements, cells were imaged and maintained under physiological conditions (5% CO2 and 37° C.) using a stage-top incubator (In Vivo Scientific, Salem, SC; Stage Top Systems). The PWS optical instrument was built on a commercial inverted microscope (Leica, Buffalo Grove, IL, DMIRB) supplemented with a Hamamatsu Image-EM CCD camera C9100-13 coupled to an LCTF (CRi Woburn, MA) for hyperspectral imaging. Spectrally resolved images of live cells were collected between 500 and 700 nm with a 2-nm step size. Broadband illumination was provided by an Xcite-120 light-emitting diode lamp (Excelitas, Waltham, MA). The spectral standard deviation (Σ) of the interference scattering originating from chromatin is calculated from the captured images. Variations in the refractive index distribution 2, can be evaluated by the mass density autocorrelation function (ACF) to calculate chromatin packing, scaling D.(29, 36, 62)

### Nuclear Volume Quantification

For nuclear volume quantification, HCT116 cells were treated with ion chelators (BAPTA-AM; APDAP-AM) for 60 minutes, fixed in 4% PFA for 10 minutes, and then stained with 4′,6-diamindino-2-phenylindole (DAPI). Fluorescence images were captured using Olympus IX-71 inverted microscope with a high-efficiency EMCCD camera (Olympus, Tokyo, Japan). The images were then analyzed using FIJI software. The nucleus was identified using binary masks, then the 3D Object Counter plugin segmented the images, the ROIs were added to 3D Manager, and lastly the ROIs were applied to the original z-stack image to determine the volume and intensity of each nucleus.

### Chromatin Electron Microscopy

### ChromEM Staining and Sample Resin Preparation

Cells were first washed with Hank's Balanced Salt Solution, without calcium and magnesium pre-warmed to 37° C. The cells were then fixed with a fixative solution prepared with 2% paraformaldehyde, 2.5% glutaraldehyde, and 2 mM calcium chloride prepared in 0.1M sodium cacodylate buffer at a pH of 7.4, for 30 minutes at room temperature and another 30 minutes at 4° C. Cells were then washed in 0.1 M sodium cacodylate buffer and then placed in a blocking buffer prepared with 10 mM glycine, 10 mM potassium cyanide, and 0.1M sodium cacodylate buffer for 15 minutes.

Nuclear DNA staining was then carried out by staining with 10 μM DRAQ5 (Thermo Fisher) in 0.1% saponin, and 0.1 M sodium cacodylate buffer for 10 minutes, covered and on ice. Then, washed ×3 with blocking buffer for 5 minutes each, followed by submerging in 2.5 mM 3,3-Diaminobenzidine Tetra Hydrochloride (DAB-HCl; Electron Microscopy Sciences, EMS). Photo-oxidation of DAB was conducted under epifluorescence with a Cy5 filter for 5 minutes with a 100× oil objective. After photo-bleaching cells were again washed ×5 for 2 minutes each. Cells were then stained with heavy metal to enhance the DNA density of DAB polymer precipitates using 2% osmium tetroxide prepared with 2 mM calcium chloride, 0.15 M sodium cacodylate buffer, 1.5% potassium ferrocyanide, for 30 minutes. After heavy metal staining, cells were washed ×5 for 2 minutes each with Millipore water.

Samples were then gradually dehydrated and embedded into Durcupan epoxy resin following standard procedures as previously described in Ou et al.(25, 29, 36). The resin-embedded samples were then processed for ultramicrotomy using an ultramicrotome (UC7, Leica) and a 35° Diatome knife, cutting the resin sample block into 120 nm sections, and laid onto slot grids with carbon/Formvar film (EMS). The samples were then coated with gold nanoparticles as fiducial markers on both surfaces.

### ChromSTEM Imaging and Reconstruction

The Hitachi 2300 STEM was used to collect HAADF images throughout a dual tilt-series collection at every 2° increments from −60° to 60. IMOD was used to align the images and reconstruction was done by Tomopy penalized maximum likelihood algorithm. IMOD was then used to combine the tomograms from two individual tilt axes.

After reconstruction, the voxel values were capped to the top and the bottom 0.1% values to remove outliers. The values were then normalized between 0 to 1 for analysis.

### Chromatin Domain Analysis:

Chromatin domains were identified and analyzed following the approach published previously(29, 36). Domain centers were selected as local maxima with prominence >1.5×standard deviation of pixel values on a 2D projected chromatin map of the tomogram after a Gaussian filter with 5-pixel radius followed by CLAHE contrast enhancement.

An 11×11 pixels window was then used to select the center pixel in each domain. The total intensity of chromatin was measured as a function of distance away from the center pixel. The power-law scaling region was identified by MATLAB ‘ischange’ function on log mass vs log radius. The domain size was determined at the point where the power-law scaling is deviated from the fitting by 5% difference, or when the local exponent D reaches 3. For the calculation of packing efficiency A, the elementary chain size Rmin was determined by a 10% difference from the power-law scaling at the lower-end of the power-law scaling region and A was calculated by A=CVC/[(Rdomain/Rmin)^{(D-3)}], where I is average intensity of the domain, R is the radius of the domain, and D is the power-law exponent.

### High Throughput Chromatin Conformation Capture

### Sample Preparation:

Cultured cells were treated with 1 μM 5-PH-IAA (MedChemExpress HY-134653) for 6 hours or with DMSO at an equivalent concentration for 6 hours(58, 59). At 6 hours, cells were harvested at 1.2×10⁶ cells per sample and transferred to a 50 mL Falcon tube, where they were centrifuged for 10 minutes at 450×g at room temperature. Supernatant was removed and cells were resuspended in 20 mL of cold fresh media. At this point, 540 μL of 37% formaldehyde was added to bring the final concentration to 1% formaldehyde and fix the cells for 10 minutes. Upon fixation, the reaction was quenched with 10 mL of 3 M Tris (pH 7.5) for 15 minutes. Cells were then centrifuged for 10 minutes at 800×g and 4° C. After resuspension at the desired concentration, individual samples were snap frozen in liquid N2.

### Hi-C Library Generation:

To wash the nuclei, samples were thawed and resuspended in 50 μL ice cold PB, followed by the addition of 150 μL ice-cold RNase-free water. 50 μL of Buffer C1 (Qiagen Epitect Hi-C Kit) was added to each sample and mixed. Samples were then centrifuged at 2500×g and 4° C. for 5 minutes. The supernatant was aspirated and the nuclear pellet was resuspended in 500 μL of RNase-free water, before being centrifuged again at 2500×g and 4° C. for 5 minutes. Library generation and subsequent steps used proprietary reagents from Qiagen's Epitect Hi-C Kit (Qiagen 59971). Washed nuclei were digested with according to the Epitect Hi-C protocol using a proprietary enzyme cocktail that cut at the GATC motif. Nuclei were end-labeled with biotin followed by ligation for 2 hours at 16° C. Ligated chromatin was then de-crosslinked using 20 μL of Proteinase K solution at 56° C. for 30 minutes and then 80° C. for 90 minutes. Ligated, decrosslinked DNA was purified using a Qiagen column kit (Qiagen 59971) and resuspended in 130 μL EB.

### Library Fragmentation:

Hi-C library samples were fragmented to a median size of between 400 and 600 bp using a Covaris E220 sonicator with a sample size of 130 μL and the settings in Table 1. Samples were purified for fragments between 400 and 500 bp using a Qiagen bead purification size exclusion kit (Qiagen 59971).

TABLE 1
Hi-C Sonication parameters

| Water levels | 12 | |
|---|---|---|
| Peak incident power | 140 | W |
| Duty factor | 10% | |
| Cycles per burst | 200 | |
| Treatment time | 55 | seconds |

### Hi-C Sequencing Library Generation:

Hi-C samples were streptavidin-purified to enrich for properly ligated contact pairs using streptavidin beads and a magnetic bead rack. Beads were first washed in 100 μL of bead wash buffer, resuspended in 50 μL of bead resuspension buffer, and then mixed with 50 μL of Hi-C sample. The mixture was then incubated at room temperature for 15 minutes in a thermal mixer at 1000 RPM. Enriched bead-bound DNA was then end-repaired, phosphorylated, and poly-A tailed using a combined ER/A-tailing solution. The samples were incubated for 15 minutes at 20° C. followed by incubation at 65° C. for 15 minutes. Beads were then washed once with 100 μL of bead wash buffer, washed again with 95 μL of adapter ligation buffer, and resuspended in adapter ligation buffer in preparation for ligation of Illumina adapter sequences. Each sample was mixed with 5 μL of one of 6 Illumina adapter sequences (specified in the Qiagen protocol appendix) and 2 μL of ultralow input ligase. These samples were then incubated for 45 minutes. Following adapter ligation, each sample was washed three times before adding 400 μL of library amplification mixture to the beads. Samples were then distributed equally across 8 wells of a 96-well PCR plate and cycled using the following parameters on an Eppendorf Mastercycler ×50s thermocycler (Table 2).

TABLE 2
Hi-C Thermocycle parameters

| | | |
|---|---|---|
| Time | Temperature | Cycle No. |

| | | | | |
|---|---|---|---|---|
| 2 | min | 98° | C. | 1 |
| 20 | s | 98° | C. | |
| 30 | s | 60° | C. | 7 |
| 30 | s | 72° | C. | |
| 1 | min | 72° | C. | 1 |

| | | | |
|---|---|---|---|
| hold | 4° | C. | hold |

Following sequencing, PCR reactions for each sample were pooled and cleaned using a Qiagen QIAseq library purification kit (Qiagen 59971). Library quality was assessed using a High Sensitivity DNA Assay on an Agilent Bioanalyzer 2100 and a Qubit dsDNA High Sensitivity Assay. Libraries were quantified using a KAPA ROX Low Master Mix qPCR library quantification kit on a QuantStudio 7 Flex instrument. Two samples per lane were sequenced on an Illumina NovaSeq 6000, generating between 400 and 600 million 150 bp paired-end reads per sample.

### Hi-C Data Processing and Analysis:

Act D treated samples and 5-ph-IAA treated samples were aligned to hg38 using Borrow-Wheeler Aligner version 0.7.17 and processed using the SLURM version of Juicer version 1.6 (github.com/aidenlab/juicer)(88) on the Quest HPC provided by Northwestern University. Processing included in the Juicer pipeline included removal of duplicates, exclusion of improperly ligated fragments, and mapping of Hi-C contacts with the GATC motif. Statistics generated for each replicate can be found in the SI. Individual replicates were checked for reproducibility using standard heuristics and combined as a mega map using Juicer's Mega script to increase sample resolution. TAD identification was generated using Juicer's Arrowhead (github.com/aidenlab/juicer/wiki/Arrowhead)(88). Loops were identified using Juicer's HICCUPS (github.com/aidenlab/juicer/wiki/HICCUPS)(88). Compartment eigenvector analysis and Pearson correlation analysis were generated using Juicer's Eigenvector and Pearsons scripts, respectively, or using built in functions in GENOVA(89). Aggregate TAD Analysis and Aggregate Peak Analysis were generated using GENOVA (github.com/robinweide/GENOVA). Contacts were dumped using Hi-C Straw (github.com/aidenlab/straw) and used for downstream analysis. Visualization was also done using GENOVA. All other analyses were custom-generated in R. All code used in this publication is available on Github at: github.com/BackmanLab.

Myoblast and myotube samples were aligned to hg38 and processed using the Next Flow Core Hi-C pipeline (github.com/nf-core/hic/tree/2.1.0). Coolers(90) were generated at 5 kb and 80 kb resolutions from the Hi-C Pro output. Contacts were dumped using Cooltools (github.com/open2c/cooltools)(91) and used for downstream analysis. Contact maps were visualized using GENOVA.

### Single Molecule Localization Microscopy

### Multi-Color SMLM Sample Preparation:

Primary antibodies rabbit anti-H3K9me3 (Abcam), mouse anti-H3K27ac (ThermoFisher), rat anti-RNA Polymerase II (Abcam) and mouse anti-H3K27me3 (Abcam) were aliquoted and stored at −20° C. Secondary antibodies goat anti-rabbit AF647 (ThermoFisher), goat anti-rabbit AF568 (Thermofisher), goat anti-rat AF488 (Thermofisher) were stored at 4° C.

Three label single molecular localization microscopy (SMLM) sample preparation is done via 3 sequential staining processed for the 3 respective targets.
1. Cells were plated on No. 1 borosilicate bottom eight-well Lab-Tek Chambered cover glass at seeding density of 12.5k. After 48 hours, the cells underwent fixation for 10 mins at room temperature with a fixation buffer composed of 3% paraformaldehyde and 0.1% glutaraldehyde in PBS. Samples were then washed in PBS for 5 minutes and then quenched with freshly prepared 0.1% sodium borohydride in PBS for 7 min. Two more wash steps were performed after quenching.
2. Permeabilization was done with blocking buffer composed of (3% bovine serum albumin (BSA), 0.5% Triton X-100 in PBS) for 1 hour and then samples were immediately incubated with rabbit anti-H3K9me3 (Abcam) in blocking buffer for 1-2 hours at room temperature and shaker. Samples were then washed three times with a washing buffer composed of 0.2% BSA, 0.1% Triton X-100 in PBS.
3. Samples were then incubated with the corresponding goat antibody-dye conjugates, anti-rabbit AF647 (Thermofisher) for 40-60 mins at room temperature on the shaker. After incubation, samples were washed two times in PBS for 5 mins on a shaker and then samples were either imaged for first labeled target or incubated overnight in a modified version of the aforementioned blocking buffer (10% goat serum+90% prior composition).
4. After overnight blocking, the samples would then go through the same protocol as in step 3 (primary and secondary antibody incubation) but this time with modified blocking buffer (90% original blocking buffer+10% goat serum) and washing buffer (99% original washing buffer+1% goat serum) for both the second and third targets. The second target primary antibody is a rat anti-RNA Polymerase II (Abcam) and the secondary antibody is goat anti-rat AF488 (Abcam). As described before, there is an overnight blocking step in between labels at 4° C. The third target primary antibody is a mouse anti-H3K27ac (Thermofisher), and the secondary antibody is goat anti-rat AF568 (Abcam). After primary and secondary incubation, the samples are washed three times with PBS and then stored at 4° C.

### Single Molecule Localization Data Analysis:

Acquired data was first processed using the ThunderSTORM ImageJ(92) plugin to generate the reconstructed images for visualization via the average shifted histogram method, as well as the localization datasets. Each localization dataset was corrected for drift and subsequently filtered such that remaining data had an uncertainty of less than or equal to 40 nm. Localization coordinates (x,y) were then used in a Python point-cloud data analysis algorithm which employed the scikit-learn DBSCAN method (parameter choice: 50 nm maximum distance between points and minimum 3 points per cluster) to cluster the heterochromatic localizations. Cluster size was determined by the area of the Convex Hull fit of the clustered marks and then normalized relative to a circular cluster with radius of 80 nm. Sample (heterochromatin, euchromatin and RNAP II) density was measured by counting the number of corresponding STORM markers in concentric rings from the identified cluster center, normalized by ring area. RNAP II association was determined by measuring the number of RNAP II that fall within 5 times expansion of the area outside of the cluster relative to all RNAP II localizations. The outside cluster condition signifies RNAP II not within any analysis area and thus not associated with heterochromatic clusters. Data shown is for concatenation of n>=4 cell replicates with approximately>500 heterochromatic clusters for each nucleus.

### Analysis of Enzyme Size

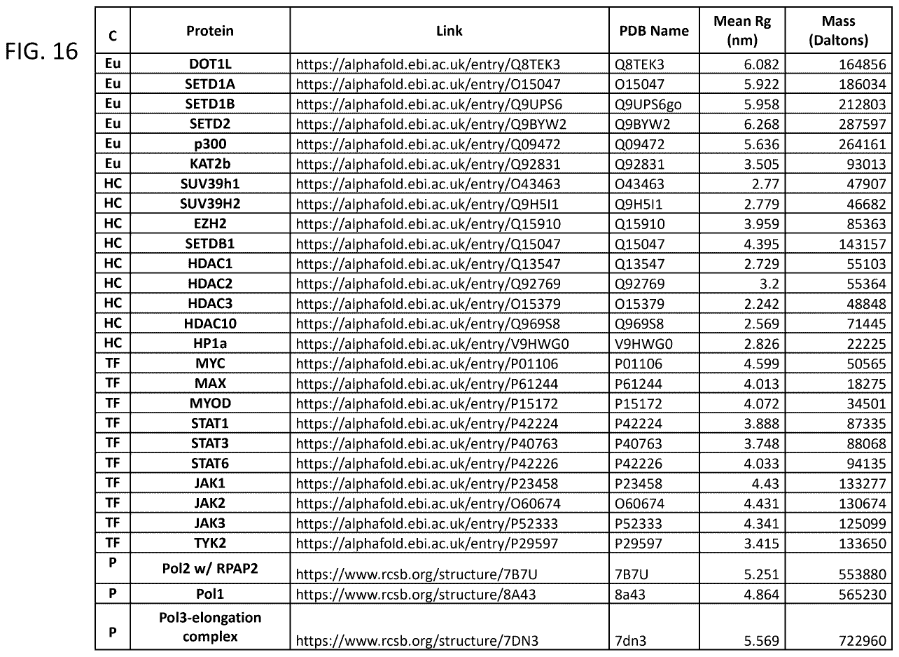

AlphaFold(50) PDB structures of transcription factors, euchromatin, and heterochromatin enzymes were obtained (FIG. 16) and the radius of gyration R_{g} was calculated from the produced atomic structures as the square root of the mean distance from the center of mass. The respective R_{g} was 4.1 nm, 5.5 nm, and 3.0 nm on average for these proteins (Table 3).

TABLE 3
Statistical testing calculated sizes from FIG. 16.

| Radius of Gyration (Angstrom) | | | | | |
|---|---|---|---|---|---|
| | Euchromatin | Heterochromatin | TTEST | TFs | Pol |
| Average | 55.61833333 | 30.52111111 | 0.000515115 | 40.97 | 52.28 |
| STDEV | 10.2878344 | 7.627439188 | | 3.61534999 | 3.53062317 |

* Statistical results from unpaired, two-tailed t-test comparing the radius of gyration of heterochromatin and euchromatin enzyme size (in angstrom). Respective size of transcription factors and RNA polymerases (in angstrom).

Statistical testing was performed on heterochromatin compared to euchromatin enzymes based on the calculated R_{g} with a two-tailed, unpaired T-Test with p-value of 0.0005. No corrections for multiple comparisons were made as these were not performed. For RNA polymerases, PDB structures were obtained from the RCSB and the R_{g} was calculated as above. As these Polymerase structures were in complex with DNA, their mass compared size was not included, however, their average R_{g} was ˜5 nm, slightly below the size of transcription factors.

### Modeling Chromatin Domains and Molecular Dynamics Study of Enzyme Penetration

The SREV configurations were obtained using the procedure described in Carignano (46). using an overall volume fraction φ=0.12 and folding parameter α=1.10 for investigation of enzyme penetration. DNA-sequences from RefSeq were superimposed onto polymer simulations from SR-EV to analyze the structure at the myosin heavy chains as a function of density with φ ranging from 0.08 to 0.20 and a fixed folding parameter α=1.15. The coordination number (CN) was calculated as the number of nucleosomes in contact with the nucleosome of interest with values ranging from 0 (isolated nucleosome) to 12 (in contact with 12 neighbors) representing various degrees of compaction. To understand the accessibility of free proteins on different regions of chromatin as a function of local volume fraction, molecular dynamics simulations of free spherical particles immersed in static SREV configurations were performed where the nucleosomes were represented as oblate Gay-Berne objects(93). To eliminate boundary effects, SREV configurations created in a cubic box of 1300 nm in size and with periodic boundary conditions were used. 10,000 spherical particles were randomly inserted in the system with no overlaps between them and with the model nucleosomes. Four different sphere size were simulated, represented by a Lennard-Jones size parameter σ=3, 6, 9 and 12 nm. The simulations were performed using the LAMMPS(94) molecular dynamics software, under NVT conditions, using a reduce length unit rᵤ=10 nm and a reduced temperature T*=2.5. The interaction parameters, described in standard LAMMPS input notation are the following:

units lj
atom_style ellipsoid

| | |
|---|---|
| set type 1 shape 1.0 1.0 0.5 | # Ellipsoids |
| set type 2 shape 0.3 0.3 0.3 | # Spheres (in this case 3 nm diameter) |

#
# pair_style gayberne gamma upsilon mu cutoff
pair_style gayberne 1.0 1.0 1.0 3.0
#
# pair_coeff ty1 ty2 eps sig (eps_a eps_b eps_c)_i (eps_a eps_b eps_c)_j
pair_coeff 1 1 1.0 1.0 1.7 1.7 3.4 0.0 0.0 0.0
pair_coeff 1 2 1.0 1.0 1.7 1.7 3.4 0.0 0.0 0.0
pair_coeff 2 2 1.0 1.0 1.7 1.7 1.7 0.0 0.0 0.0

### ChIP-Seq Analysis

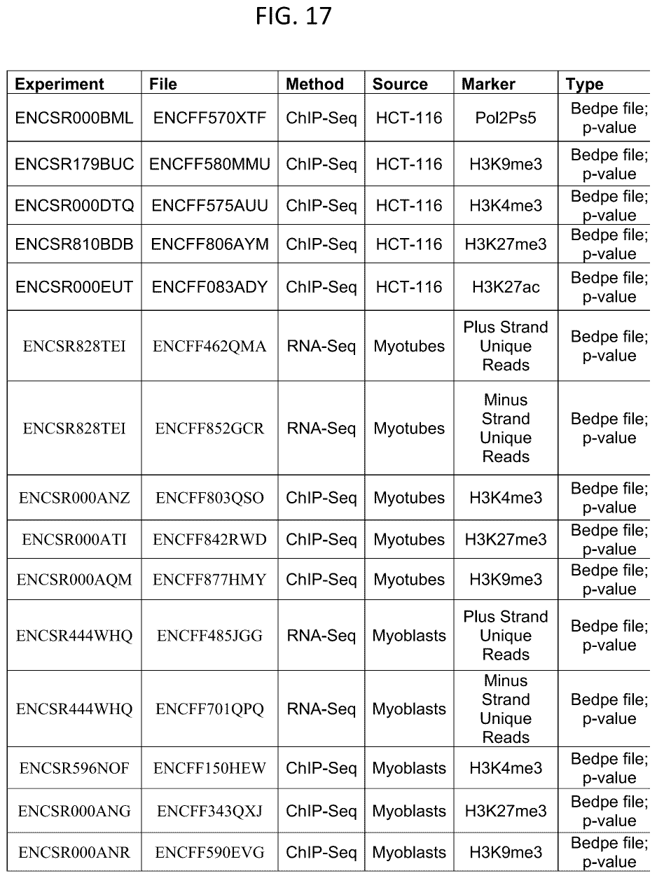

Utilizing data available through ENCODE(67-69) (FIG. 17), the relationship between serine-5 phosphorylated RNA polymerase (PolII-PS5, initiation of transcription)(65) to epigenetic marks was analyzed as follows. RefSeq gene positions were consolidated to a single, longest defined start/stop position of the reported isoforms of the same gene annotation. Following consolidation, the mean location of each peak for the different marks (FIG. 17) were identified such that their p-value was <0.1. Genes were then organized by the cumulative number of PolII-PS5 peaks within each gene from 1 to 15 in HCT-116 cells. The absolute linear distance from each PolII-PS5 to the nearest chromatin mark was then calculated. The cumulative density function of distances was used to identify the average distance as a function of the number of identified peaks with a linear regression fit to the resulting value as shown. With respect to the per chromosome analysis, the total segment length of each mark was calculated and marks were compared to the coverage of PolII-PS5 across the somatic chromosomes with the exclusion of X and Y chromosomes from this analysis due to their distinct mechanisms of heterochromatin formation.

### Phenomenological Loop Domain Formation Model

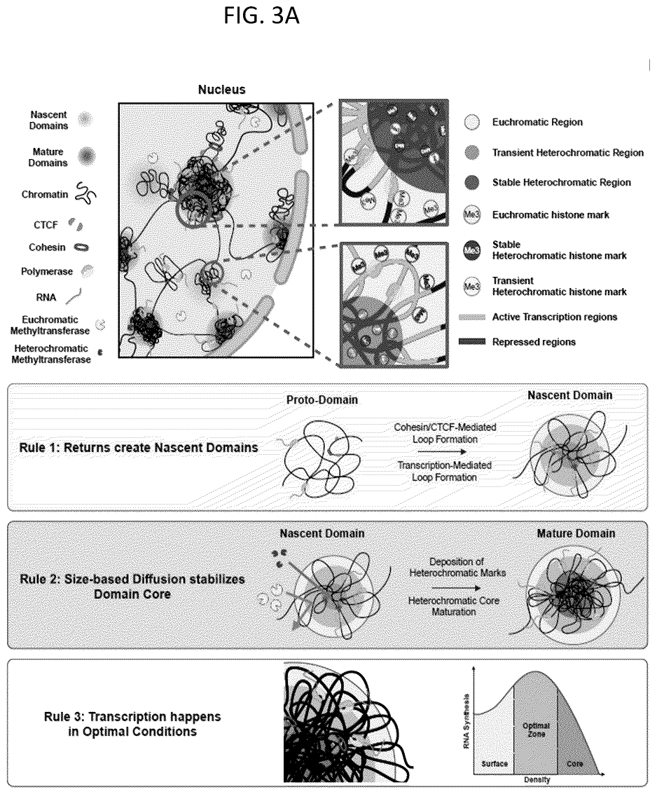

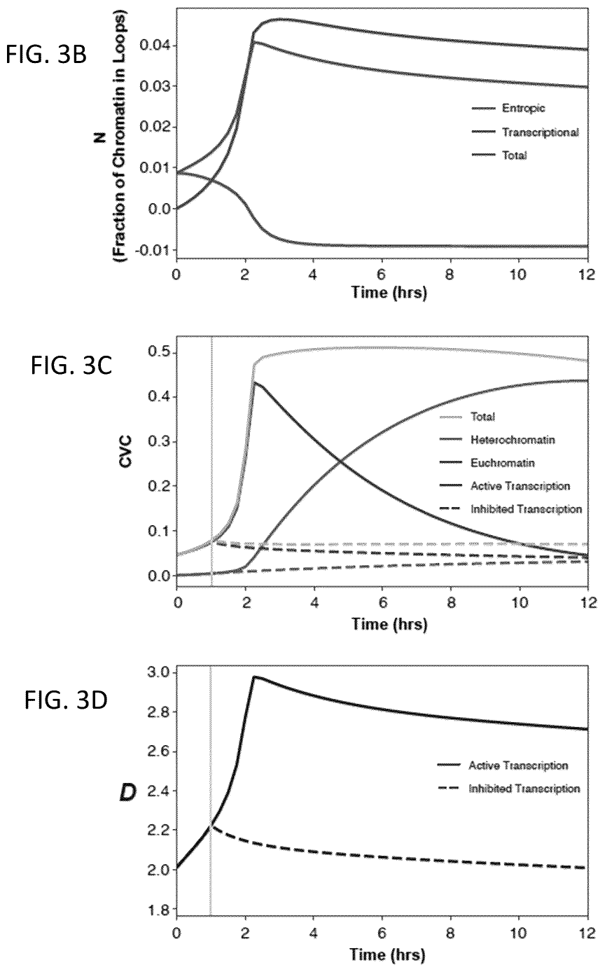

A computational model was developed to describe 3 primary rules that regulate the polymeric structure-function of chromatin domains: (1) Returns (loops) create local areas of high density from long-range confinement (2) nucleosome remodeling enzyme localization depends on their excluded volume interactions with polymeric domains, and (3) transcriptional reactions non-monotonically depend on local crowding conditions from the competition between enzyme diffusivity and the free-energy of the chemical reactions resulting in a zone of improved efficiency (FIG. 3A). For simplicity, transcription is described as the primary generator of returns. One could also include cohesin mediated loop extrusion as a second process, but the consequence is like transcription alone.

First, basic physical properties of a packing domain formed from polymeric folding of chromatin in nuclear space are defined. φ is defined as the chromatin volume fraction (CVC within the main text), the space occupied by chromatin in the theoretical volume. φ=0 denotes a volume free of chromatin and φ=1 represents a fully filled volume occupied by the chromatin polymer. Since chromatin behaves as a power-law polymer at domain length-scales, φ directly relates to folding properties that are infrequently utilized in the context of gene regulation. From prior work in ChromEMT, the smallest element of the chromatin polymer chain, rₘᵢₙ, is ˜10 nm. The density of chromatin within a volume can then be described by from the relationship from rₘᵢₙ by the scaling exponent, D, and the efficiency with polymer fills this space A (the packing efficiency).

### Rule 1:

Within a domain volume, one can consider loops per unit volume as quantizable events (‘loopons’), N, as a function of their spatial and genomic distance. In a relaxed polymer without returns or confinement, the likelihood of two segments of a polymer in contact decays exponentially as a function of the linear genomic separation, X, by the contact-scaling exponent, s. In the context of a crowded nucleus with enzymatically mediated but infrequent looping events, loop events arise stochastically from confinement or from transcription creating quantized events along a segment that decreases the distance. Contact scaling of the segments as a function of genomic distance depends on the frequency of loop events by

$$s \approx ln ( \frac{{N}_{0}}{N} ) / ln ( \frac{X}{{X}_{0}} ) .$$

From imaging experiments and polymer modeling, contact scaling relates to D by s˜1/D (which has been described extensively in Li et al(29)). We consider a fractal random walk polymer approximate for which s˜3/D. Thus, it arises that the effect of loop frequencies in a nuclear volume are quantifiable by:

$$1. D ≅ 3 * ( log ( \frac{X}{{X}_{0}} ) / log ( \frac{{N}_{0}}{{N}_{Opt}} ) ,$$

where X₀ is the base genomic chain length of and N₀ is the base loop volume fraction. It is reasonable to start with a distance ratio (X/X₀) of ˜24 (gene length on average ˜24 kbp, chain length ˜1 kbp) and use N₀˜1 to indicate that each base pair cannot be split between two separate loops. From this relationship, the N_{Opt}, the improved loop capacity for a given set of conditions (ex. monomer-monomer and monomer-solvent interactions) within the volume, can then be calculated. The change in the domain D over time, D(t), depends on the evolution of the total number of loops over time, N(t) and their change over time as follows:

$$2. \frac{d D ( t )}{d t} = \frac{{N}^{′} ( t )}{N ( t )} \times \frac{D ( t )}{log ( N ( t ) / {N}_{0} )}$$

In principle, N can be positive or negative, as it quantizes loop behavior along a chain to describe loop formation and loss. The total N is the sum of the loop volume fraction from enzymatic processes (primarily transcription; Nₜ) and entropic events (N_{Δs}):

$$3. N = {N}_{t} + {N}_{Δ s} .$$

While N is generally positive, Nₜ and N_{Δs} can be negative representing the elimination of previously formed loops. From the reasonable assumption that transcription can only occur when engaged in euchromatic segments, φₑ, active transcription results in the formation of returns that are actively removed via the family of topoisomerases (TOPs). Consequently, loops mediated by transcription evolve by:

$$4. \frac{d {N}_{t} ( t )}{d t} = {ϕ}_{e} ( t ) {Γ}_{POL 2} - {N}_{t} ( t ) {Γ}_{TOP}$$

Here, Γ_{POL2} denotes the rate of transcriptional mediated loop formation and Γ_{TOP} is the rate at which topoisomerase degrade these loops.

The formation and degradation of entropic loops can similarly be incorporated through the interplay between improved loop capacity N_{Opt} and the current loop occupancy N:

$$5. \frac{d {N}_{Δ S}}{d t} = {Γ}_{Δ S} * ( {N}_{Opt} \to N ( t ) ) ,$$

with Γ_{Δs} being the rate of entropic loop formation and decay, which captures the process by which loops evolve independent of protein mediated extrusions. From equations 3-5, the time evolution of loop formation can be modeled and equations 1 and 2 model the formed domain structure.

### Rule 2:

Having described how N intersects with D in domains, the relationship between D, φ, and A can be used to describe the temporal evolution of the space-filling properties of domains. Chromatin is frequently described by the functional modifications into heterochromatin (poorly accessibly, φₕ) and euchromatin (highly accessible, φₑ). The total φ is therefore the sum of euchromatin and heterochromatin in the theoretical volume, φ=φₑ+φₕ. Considering that chromatin volume concentration decreases with radial distance r from the center of a chromatin domain as (r/rₘᵢₙ)^{D-3} and restricting the focus in time during the time-scales of return formation through possible degradation, the temporal evolution of domains and their average volume fraction are quantified by:

$$6. ϕ ( t ) = {ϕ}_{e} ( t ) + {ϕ}_{h} ( t ) = \frac{3}{D ( t )} \times A ( t ) {( \frac{{r}_{min}}{t} )}^{3 - D ( t )} .$$

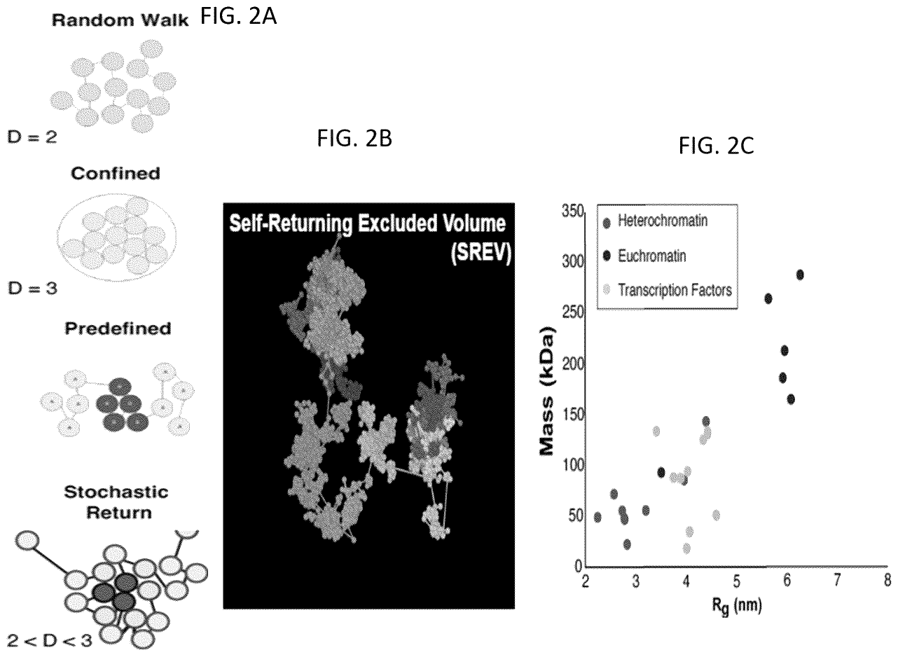

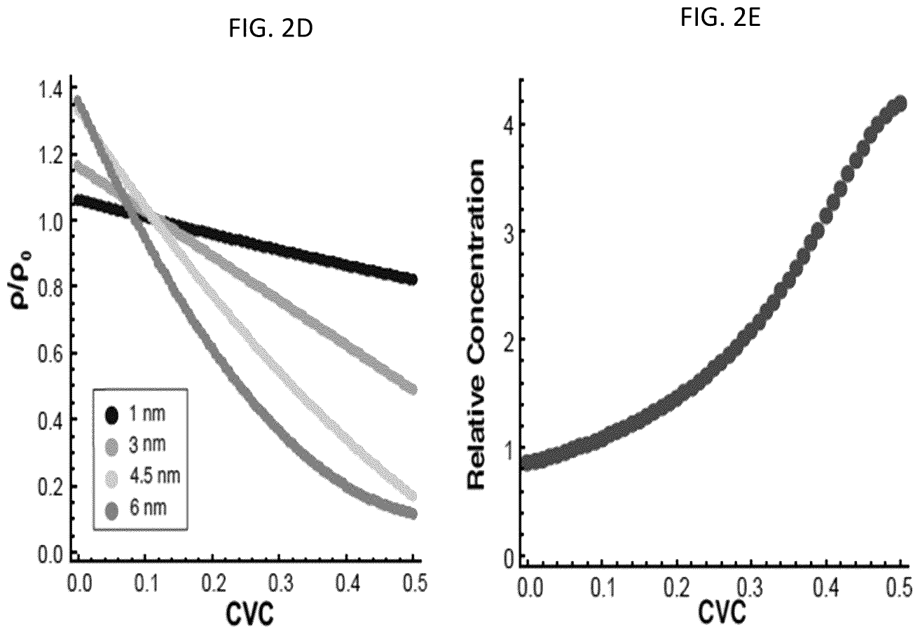

From Eq. 6, the temporal evolution of φ in relation to the polymeric folding of the chromatin in the limiting case of a perfectly space-filling, efficiently packed polymer A=1 and D=3 converges as expected to the value of 1 (fully compacted). Likewise, φ(t) will converge to A(t) in the limiting case of D=3 or to 3*A(t)/D(t) for r=rₘᵢₙ. From ChromEMT data(25), a heterochromatin chain is more tightly packed, therefore A is proportionally related to the fraction of euchromatin and heterochromatin. A of a domain that is fully euchromatic is defined as Aₑ and fully heterochromatic is defined as Aₕ. Experimentally, domains are observed to exist with A=1 and are therefore presumably fully heterochromatic chains result in Aₕ=1. A can then be extrapolated for intermediate fractions of euchromatin and heterochromatin as follows:

$$7. A ( t ) = ( ( {ϕ}_{h} ( t ) + {A}_{e} * {ϕ}_{e} ( t ) ) / ( {ϕ}_{h} ( t ) + {ϕ}_{e} ( t ) ) ) ,$$

where Aₑ is less than 1. Chromatin modifying enzymes have a distinct distribution of sizes that correlates with their post-translational modification of nucleosomes (FIG. 2B). From SR-EV simulations (FIGS. 2C and 2D), enzyme size results in a spatial preference for heterochromatin enzymes localizing within a domain interior whereas larger euchromatin enzymes and transcription factors favor the periphery (FIGS. 2C and 2D).

To model this, a generalized reaction rate equation was created based on prior work studying the interactions of excluded volume with transcription reactions for each reactant group: RNA polymerase (Γ_{POL2}), topoisomerase (Γ_{TOP}), heterochromatic modifiers (Γₕ), and euchromatic modifiers (Γₑ). As an approximation, this can be captured by:

$$8. {Γ}_{x} = {Γ}_{x} ( t ) = {B}_{x} * ϕ * ( 1 - \frac{ϕ}{{σ}_{x}} )$$

Eq. 8 is then used to account for differences in activity based on the likelihood of localization of the enzyme and scaled relative to the size of Pol-II(47). This is modeled by the rate coefficients, Bₓ, to account for differences in protein activity as an initial approximation, as it is expected that some proteins have different rates of activity based on their size in diffusion limited reactions with smaller proteins have a greater Bₓ than larger ones. Naturally, there is a φ at which the protein can no longer diffuse or perform its function due to its size, which is denoted as σₓ. The values of Bₓ and σₓ can then be used for each type of protein type for this model can be found in FIG. 16 and depends on the findings from the SR-EV simulations in FIG. 2.

The change in domain structure from these enzymatic processes is thus quantified by:

$$9. \frac{d {ϕ}_{h} ( t )}{d t} \to \frac{d {ϕ}_{e} ( t )}{d t} = {ϕ}_{e} ( t ) {Γ}_{h} ( t ) - {ϕ}_{h} ( t ) {Γ}_{e} ( t ) ,$$

which captures the change in domain density from the rate of compaction of euchromatin into heterochromatin via heterochromatin modifying proteins (SUV39H1/2 or histone deacetylases) and the rate of decompaction of heterochromatin into euchromatin.

Over time, domain core densities will exclude larger proteins and only allow the transit of small molecules or heterochromatin proteins. As a result, domain interiors become favorable for cross-linking reactions from small proteins such as HP1a. To model this process in relation to the rate of entropic relaxation, it is assumed that HP1a crosslinking is a function of local density by, ps, and counteracts the maximal entropic rate B_{Δs}:

$$10. {Γ}_{Δ S} = {B}_{Δ S} * {e}^{( \frac{- ( {ϕ}_{h}}{Φ * ps} )} .$$

Even as domains have variations in their density radially, the probability distribution function of φ for these molecules in a domain is captured by:

$$11. 〈 {Γ}_{x} ( r ) 〉 = {\int }_{P D} {Γ}_{x} ( ϕ ) \times pdf ( ϕ ) d ϕ .$$

Extending this into the probability distribution function in a power-law domain, it is observed that the

$$pdf ( ϕ ) ∝ {( \frac{{r}_{min}}{r} )}^{3 - D} \times {r}^{2} . \qquad 12.$$

For the purposes of simulations, this can be expanded into the following analytic form:

$$\{ \begin{matrix}〈 {Γ}_{x} 〉 = {B}_{x} * A * D * {( \frac{{r}_{min}}{r} )}^{D} * \\ ( \frac{1}{2 D - 3} * {( \frac{{r}_{min}}{r} )}^{D} - \frac{1}{3 * ( D - 2 )} * \frac{A}{{ϕ}_{c}} * {( \frac{{r}_{min}}{r} )}^{3} ) * ( {( \frac{{r}_{min}}{r} )}^{3 - 3 D} ) + \\ ( \frac{1}{3 * ( D - 2 )} - \frac{1}{2 * D - 3} ) * {( \frac{A}{{ϕ}_{c}} )}^{( ( 3 - 2 * D ) / ( D - 3 ) )}\end{matrix} \} . \qquad 13.$$

Rule 3: Excluded volume within the nucleus impacts the transcription rate such that the resulting relationship of mRNA concentration as a function of φ has a non-monotonic behavior with a peak centered around φ˜0.2 to 0.35 depending on the binding efficiency of transcription reactants, the dissociation constants, and the efficiency of RNA polymerase synthesis. Crowding produces the observed nonmonotonic behavior from two competing processes: (1) crowding without attractions slows molecule diffusion as a function of size and density; (2) excluded-volume facilitates stabilization of intermediary complexes by increasing the entropy of the system. This region of ideal density is defined as the “goldilocks” zone, φ_{GL}:

$${ϕ}_{GL} = A * {( \frac{{r}_{GL}}{{r}_{min}} )}^{D - 3} . \qquad 14.$$

In turn, this can be converted to identify the radius where ideal conditions occur:

$${r}_{GL} = {r}_{min} * {( \frac{{ϕ}_{GL}}{A} )}^{( 1 / ( D - 3 ) )} . \qquad 15.$$

Within this disclosure, the solved equations are as follows:

$$N = {N}_{t} + {N}_{Δ s} ; \qquad 3.$$

$$\frac{{dN}_{t} ( t )}{dt} = {ϕ}_{e} ( t ) {Γ}_{POL 2} - {N}_{t} ( t ) {Γ}_{TOP} ; \qquad 4.$$

$$\frac{{dN}_{Δ s}}{dt} = {Γ}_{Δ s} * ( {N}_{Opt} - N ( t ) ) ; \qquad 5.$$

$$ϕ ( t ) = {ϕ}_{e} ( t ) + {ϕ}_{h} ( t ) = \frac{3}{D ( t )} \times A ( t ) {( \frac{{r}_{min}}{r} )}^{3 - D ( t )} ; \qquad 6.$$

$$\frac{d {ϕ}_{h} ( t )}{dt} - \frac{d {ϕ}_{e} ( t )}{dt} = {ϕ}_{e} ( t ) {Γ}_{h} ( t ) - {ϕ}_{h} ( t ) {Γ}_{e} ( t ) ; and \qquad 9.$$

$${Γ}_{Δ s} = {B}_{Δ s} * {e}^{( \frac{- ϕ}{ϕ * ps} )} . \qquad 10.$$

And the following initial conditions:
D(0)=2.01, Nₜ(0)=0, N_{Δs}(0)=N_{Opt}, and φₕ(0)=0 where N_{Opt} will depend on D. When solving for D_{pd}, Nₜ, N_{Δs}, φₑ, and φₕ, simulations were run until N reached a derivative of zero, indicating that the system had reached a steady state. Remaining parameters are present within Table 4.

TABLE 4
Parameters used in phenomenological
loop domain formation model.

| FIXED | | | |
|---|---|---|---|
| PARAMETERS | DESCRIPTION | VALUE | |
| A_{POL2} | Scaling factor for the max | 2 | |
| | rate of RNA Polymerase II | | |
| A_{TOP} | Scaling factor for the max | 0.5 | |
| | rate of Topoisomerase | | |
| Aₕ | Scaling factor for the max | 1 | |
| | rate of heterochromatic | | |
| | modifiers | | |
| Aₑ | Scaling factor for the max | 1 | |
| | rate of euchromatic modifiers | | |
| A_{Δs} | Scaling factor for the max | 1 | |
| | rate of crosslinking proteins | | |
| σ_{POL2} | Chromatin volume fraction | 0.5 | |
| | limit in which RNA | | |
| | Polymerase II can no longer | | |
| | diffuse | | |
| σ_{TOP} | Chromatin volume fraction | 0.7 | |
| | limit in which Topoisomerase | | |
| | can no longer diffuse | | |
| σₕ | Chromatin volume fraction | 1 | |
| | limit in which | | |
| | heterochromatic modifiers | | |
| | can no longer diffuse | | |
| σₑ | Chromatin volume fraction | 0.5 | |
| | limit in which euchromatic | | |
| | modifiers can no longer | | |
| | diffuse | | |
| ps | Crosslinking strength of the | 0.1 | |
| | PD core | | |
| rₘᵢₙ | The diameter of the smallest | 10 | nm |
| | element in a chromatin chain: | | |
| | a nucleosome | | |
| r | The average radius of a PD | 150 | nm |

| | (experimental value attained | |
|---|---|---|
| | by ChromSTEM) | |
| X/X₀ | Ratio of genomic distance | 24 |
| N₀ | The loop volume fraction for | 1 |
| | the smallest element of the | |
| | chromatin chain | |
| Nₘₐₓ | The optimal loop capacity | 0.0087 |
| | within the volume | |
| φ_{0, e} | Proportion of the chromatin | 0.45 |

chain that is euchromatic

### Results

### Chromatin Packing Domains are the Predominant Supra-Nucleosome Nuclear Structure

The pioneering work by Ou et al(25) described the capacity to proportionally label DNA utilizing a photo-activatable dye (DRAQ5) and Click-chemistry of diaminobenzidine (DAB) that results in specific staining of DNA on electron microscopy (ChromEM)(25). This approach overcomes a major prior limitation in chromatin electron microscopy, as in prior studies the non-specific binding of contrast resulting in binding to chromatin and non-chromatin molecules. Using ChromEM, it was shown that individual DNA fibers and nucleosome assemblies form as disordered nucleosome clutches (5-25 nm)(25). To extend the capabilities of ChromEM technologies to higher order structures, scanning transmission chromatin electron microscopy (ChromSTEM) with high-angle annular dark field tomography was developed. In ChromSTEM tomography, the mass density of DNA is proportional to the intensity with a resolution ˜2 nm. Although ChromSTEM tomography is the only method capable of resolving the ground-truth physical structure of chromatin, the throughput is limited and it's not currently possible to delineate DNA density at specific genes. By pairing ChromSTEM with other modalities as described below some of these limitations can be overcome. Using ChromSTEM tomography, chromatin is demonstrated to form into higher-order structures from these disordered nucleosome fibers (<25 nm) by folding into heterogeneously distributed packing domains (PD, 50-200 nm), and finally converging into space-filling territories (>200 nm)(29, 36). Physically, PDs are heterogeneous power-law chromatin assemblies with a distribution of densities, sizes, and folding properties.

Several properties of packing domains are obtained with ChromSTEM tomography: average density also known as the chromatin volume concentration (CVC), domain size (radius, r), the polymeric filling of chromatin within the domain as quantified by the mass fractal dimension, D, and the packing efficiency (how efficiently nucleic acids fill the domain volume)(36). While domain radius and CVC are relatively intuitive properties (how large and how dense a domain is, respectively), fractal dimension and packing efficiency are not frequently described metrics of chromatin biology. However, these properties are important to translate how the polymeric structure of chromatin intersects with the occupied volume, as described below.

Consider a segment of the genomic length, L, e.g. 100 Kbp, comprised of nucleosome monomers linked together by DNA. Although each nucleosome likely has slight variations in DNA content, for simplicity, it is reasonable to assume that these contain ˜200 bp. This would produce a linear segment of ˜500 nucleosomes. Contact probability between monomers decays as a function of the segment length if no constraints are present with contact scaling S, a metric used in polymer models and experimental methods such as Hi-C(6, 9, 37, 38). This indicates that adjacent nucleosomes on this segment are more likely to be in contact than those further away (e.g., nucleosome 250 is more likely to contact the 240/260 positions than the 100/400 positions). This same segment of chromatin has an accompanying total number of nucleotides, M, that occupies a domain volume with radius, r by the mass-to-distance relationship, M(r)∝Ar^{D}. Packing efficiency, A, is a complementary measurement that ranges between 0 and 1, where 1 indicates that the domain volume is optimally filled(36).

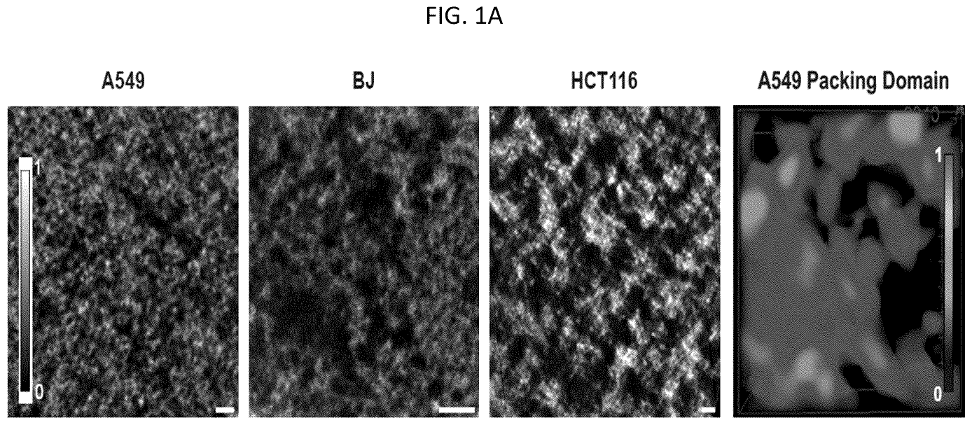

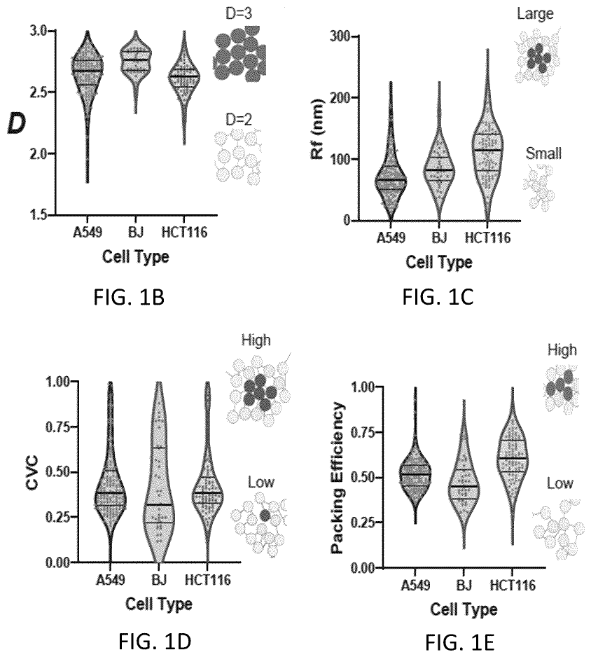

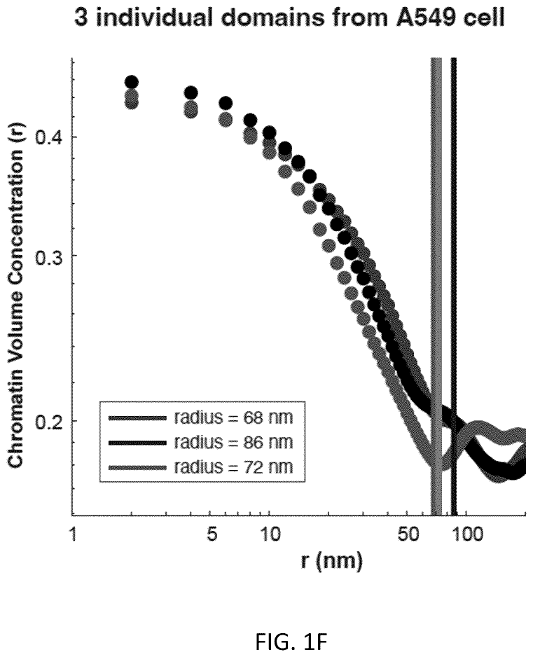

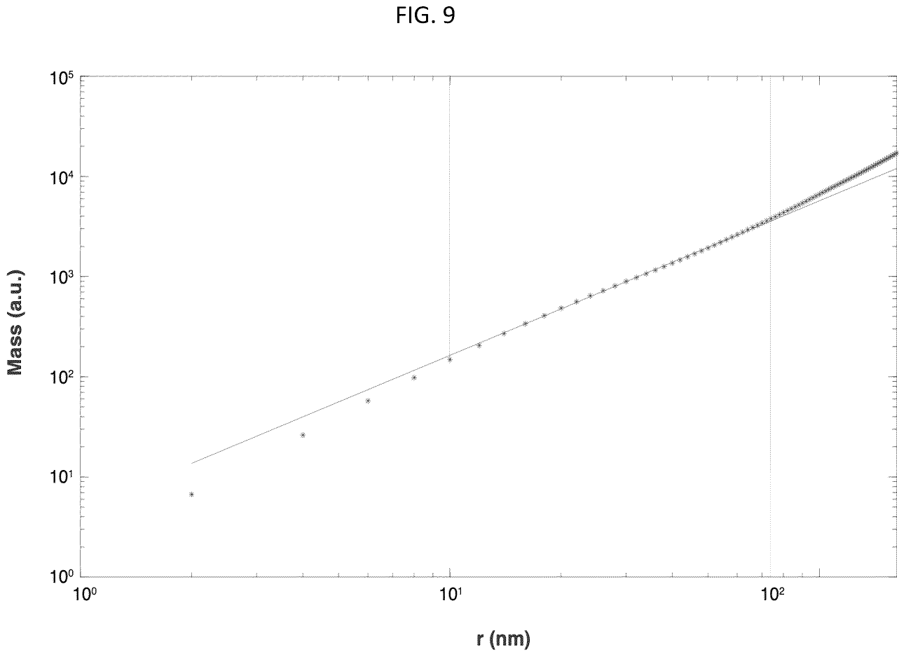

Independent of the cell line (A549, HCT-116, and CRL-2522 fibroblasts), PD formation was observed to occur (FIG. 1A) with distinct distributions in D (FIG. 1B), domain radius (FIG. 1C), chromatin volume concentration (CVC; FIG. 1D), and packing efficiencies (FIG. 1E). In the majority of cases, the domains had D>2. Visually, PDs have a high-density interior that decays to a low-density exterior (FIG. 1F, FIG. 9) in all tested models, until the emergence of the transition to inter-domain areas (FIG. 1F) with CVC decreasing as a function of distance r from the domain center as CVC∝A/r^{3-D}, a relationship that follows from the mass scaling above and confirmed by ChromSTEM. Interestingly, the CVC at domain edges typically approaches ˜20% (FIG. 1F), indicating domains transition not from high density to very low density but toward intermediate physical conditions. A remarkable feature of domains is that they defy discrete binarization into high-density and low-density structures. As a result, domains do not appear to represent assemblies of two distinct chromatin phases but a continuous distribution of states. Consequently, these domains are best defined not by high internal density but by the power law scaling internal conformation of the chromatin polymer, i.e., conformationally defined domains.

Pairing the information of domain size and packing efficiency, it was expected that although ChromSTEM imaging is performed on fixed cells, in principle the cross-section of observed domains provides information on their lifecycle. Specifically, it was expected that high packing efficiency could be consistent with mature domains; large, low packing efficiency domains could be consistent with decaying domains; and small, low packing efficiency domains could be consistent with newly forming (nascent) domains. Collectively, this suggested that domains existed in a unified, dynamic domain forming system, but this could not be delineated with electron microscopy experiments alone. As explored below, pairing domain states with molecular interactions is facilitated by integration of mathematical modeling, polymer simulations, and nanoscale molecular imaging.

### Long Range Chromatin Interactions, Nuclear Density, Excluded Volume, and Ionic Interactions Influence the Structure of Domains.

Since domains did not separate into two dichotomous groups, what processes could produce a continuous distribution of structures was next considered. To do so, polymer modeling was used. The simplest polymer model of a chromatin segment is a random walk (FIG. 2A). Each nucleosome monomer is linked by a fixed distance and two nucleosomes cannot occupy the same space (FIG. 2A). An interesting observation of this model was that, without any other constraints, it can only statistically produce a fractal dimension of D=2. This is a consequence of the Central Limit Theorem: the sum of N steps with independent jump vector distributions having a finite expectation and a variance is normally distributed with the variance proportional to N; this ensures that the spatial extent of the random walk (e.g., the radius of gyration) scales as ˜N^{1/D} with D=2(37). In contrast, most packing domains observed by ChromSTEM have 2<D<3. Several modifications have been attempted to overcome this limitation. One approach is to produce physical confinement, which produces the fractal globule which is a limiting case where D=3 (FIG. 2A)(6, 37). In both approaches, it is not possible to produce the domain states observed on ChromSTEM while maintaining the visually apparent corrugation (gaps between domains, FIG. 1A). An alternative strategy is to apply attractive potentials between different monomer segments (A attracts A, B attracts B) using a priori segment information and would produce dichotomous segments without a continuous mass-density decay (FIG. 2A)(9, 11-13, 38-41).

an alternative approach centered on the concept of a stochastically returning random walk (SRRW) was previously described (42). In SRRW, ‘returns’ are a mathematical concept stating that a segment has a probability, p, of returning to a prior point and a probability 1−p of taking a forward step to a subsequent point. In SRRW, step sizes are not a single fixed distance. Instead, the size of any step was modeled as an inverse power law distribution where short steps were more likely, but long-steps still rarely occurred. Finally, the likelihood of a return or forward step itself depended on the distance traversed in the current step. The longer the current step, the less likely for a return to occur (FIGS. 2A and 2B). The main limitation in SRRW is the segments do not occupy a physical volume (they are dimensionless). In SRRW, therefore, segments cannot interact with one another, chromatin remodeling enzymes, or polymerases. In SRRW, physical attributes such as the nuclear volume, do not impact the nodes and branches(42). This limitation was addressed recently by turning this mathematical framework into a polymer model where each segment was modeled as a nucleosome disk, the so-called stochastic returns with excluded volume (SR-EV) model(42) (FIGS. 2A and 2B). SR-EV retains the mathematical framework of SRRW, but since the segments are now space-filling nucleosomes, it is easier to interpret what steps and returns represent in the context of biological processes and chromatin structure.

In eukaryotic nuclei, a nucleosome can interact with neighboring nucleosomes due to a combination of short and long-range processes. These include the activity of heterochromatin- and euchromatin modifying enzymes, nucleosome-nucleosome bridging (e.g. HP1 crosslinking), cohesin extrusion(23, 43, 44), transcription mediated promoter-promoter (P-P) interactions(28, 41), (3) promoter-enhancer (P-E) interactions(43), and (4) stochastic contacts from confinement of the genome in 3D space(45). The distribution of steps produced by these factors implicitly enters into the model at the level of the steps and returns produced. Some processes (loop extrusion, P-P, and P-E) are unique in that they may produce forced long-returns but it is not necessary to explicitly define the position of forced returns to reproduce the domains observed on ChromSTEM. This stochastically-forced returns excluded volume (SR-EV) has a remarkable degree of quantitative and qualitative agreement with domains observed on ChromSTEM(46) (FIG. 2b), recapturing the distribution of sizes, CVC, and D values(46). As explored below, when pairing these considerations with the physical properties of nucleosome remodeling complexes (heterochromatin enzymes are small, euchromatin enzymes are large) it has profound implications on the role of domains in transcriptional reactions.

It is immediately clear that without pre-defining monomer-monomer attractive forces or fixed loop extrusion as barrier elements, SR-EV creates the domain structures observed above in FIG. 1(46). Two features from SR-EV and ChromSTEM tomography (FIG. 1 and FIG. 2) are immediately evident (1) domains are the predominant chromatin physical structure and (2) they are not produced by dichotomous segmentation into hetero- and eu-chromatin. An approach with dichotomous segmentation would not produce the continuous, mass-fractal behavior of domains (density gradually decaying from the center as an inverse power law of the radial distance∝1/r^{3-D}) with 2<D<3 but would instead create discrete partitions. Instead, stochastically encoded forced returns are needed for domains to form with the experimentally observed geometry. The fact that forced returns are important for domains to form is evident from the Central Limit Theorem for dependent random variables: forced returns result in anticorrelation between steps that might be separated even by a large linear distance N. This results in an attenuated dependence of the variance of the sum of N steps as a function of N. While in the absence of forced returns, the spatial extent of a random walk ˜N^{1/2}, forced returns allow for the ˜N^{1/D} scaling with D>2. In the absence of such a process, mass-fractal domains will not form (FIG. 2a).

Since forced returns may generate nascent domains, it is worth now considering the structure of nucleosome remodeling complexes, cohesin subunits, and RNA polymerases. All of these are known to be multi-subunit protein complexes and as such, they also occupy space in the nucleus. While it is difficult to know if these multi-subunit complexes assemble in situ onto DNA sequences or are pre-assemble within the cytoplasm, as a first approximation the effect of enzymes size in how it interacts with domains was investigated.

Building on the excluded-volume concepts introduced by Matsuda et al(47), Putzel et al(48), Maeshima et al(49), and Miron et al(24) in chromatin, molecular size can define what areas are accessible and inaccessible. This principle is widely used in molecular biology techniques like Western blots where a 1 nm protein can more easily and quickly traverse the gel than a 10 nm protein when subjected to an electric field with equivalent charges. In contrast to a gel, chromatin domains are heterogeneous structures and can undergo chemical reactions with the molecules they interact with (e.g. transcription). Utilizing AlphaFold, protein mass was converted for these nuclear enzymes into approximate protein sizes(50). From the AlphaFold predicted configurations of heterochromatin enzymes, euchromatin enzymes, and transcription factors, the radius of gyration (R_{g}) was calculated of each protein and found that on average, euchromatin enzymes were significantly larger than heterochromatin enzymes (R_{g} of ˜5.5 nm in comparison ˜3 nm) FIG. 2C, p-value <0.001; FIG. 16) with EZH2 (R_{g} of 3.95 nm), which catalyzes the formation of H3K27me3 as an interesting outlier.

Using configurations generated by SR-EV, the relative penetration of a 1.5 nm small molecule, a 3 nm “heterochromatin” protein, a 4.5 nm transcription factor, and a 6 nm “euchromatin” protein was measured as a function of domain density. This intrinsic property when paired with SR-EV configuration results in spatial preferences of larger, “euchromatin” proteins to the domain periphery with preferential localization of smaller “heterochromatin” proteins to the dense domain interiors (FIG. 2d). Indeed, heterochromatin enzymes were much less likely to be found at the periphery of domains, with a fourfold abundance in their relative concentrations at high density interiors compared to euchromatin ones (FIG. 2e). The RNA polymerase core subunit, interestingly, has an R_{g} of ˜5 nm that is comparable to euchromatin enzymes (FIG. 16 and Table 3). It would likely localize in the inter-domain space when it is inactive. However, a relevant attribute of transcriptional machinery is that it operates best with an intermediate degree of crowding. This occurs from competition between the entropic gain of the polymerases remaining bound to DNA during intermediate reactions and the diffusion rates of the reactants. The low chromatin density outside of a domain increases the diffusion rates of transcriptional reactants at the expense of their binding constants; the high-density chromatin cores are inaccessible to most reactants, whereas the “ideal” conditions at the intermediate periphery of a mature domain optimize both rate limiting processes (diffusion, intermediate reaction complex stability) due to the excluded volume effects(47, 51-53). As such, the improved conditions for transcription reactions are not on the outer zone, but within an intermediate ideal physical zone(29, 47, 52-54).

Finally, a consideration that arises from the observation of high-density centers within domains is the regulation of the electrostatic charges from the large concentration of DNA within domain interiors. Functionally, the post-translational modification of histone proteins helps to buffer these charges in conjunction with the local ion concentrations (Na⁺, K⁺, Ca²⁺, Mg²⁺, etc.)(55-57). As such, manipulation of ion concentrations would also be expected to exert an effect on domain stability with the loss of divalent ions (Ca²⁺, Mg²) anticipated to result in domain collapse.

### a Phenomenological Model of Chromatin Domain Self-Assembly

Motivated by these observations, it was expected that a framework based on the domain life-cycle could explain the paradox of why inhibiting heterochromatin enzymes can disrupt transcription. It was postulated that the domain life cycle depended on three rules that intersected with transcriptional reactions. (1) Long steps from a forced return (cohesin, P-P, and E-P interactions) creates nascent domains. This occurs because a local density pocket is formed that results in some preferential positioning of hetero- and euchromatin enzymes but not to the extent observed in mature domains. (2) Domain maturation depends on the preferential localization of enzymes within centers, ideal zones, and peripheries. Maturation is not guaranteed to happen from a nascent domain unless a critical mass is initially reached to produce the preferential position of hetero- and eu-chromatin enzymes. (3) Transcription accelerates at the formed ideal physiochemical zone due to the stabilization of the intermediate complexes (FIG. 3a). Once active transcription is entrenched, domain boundaries arise from the polymerase acting as a barrier in the ideal zone to prevent domain swelling due to its preferential function in this space.

To couple these processes into testable predictions of packing domain structure-function, a mathematical model of these processes was developed. This model pairs the mass-fractal physical properties of domains in their-life cycle with the reaction processes above (see Table 4 model for derivations). Capturing the mass-fractal-like properties of domains is important, as these properties generate a functional ‘interface zone’ that is absent in domain models with solid or condensate structures. In condensate models, the interface is a negligible portion of the structure, reducing its functional capacity. In contrast, the mass-fractal structure on ChromSTEM allows for a large interface area where reactions can occur (FIG. 1).

One can reasonably start with a relaxed segment of chromatin in a confined nucleus without nucleosome modifications as this may reflect a sufficiently large segment of chromatin as the cell exits mitosis. Nascent domains could be formed by 3 processes capable of generating forced returns: transient contacts from spatial confinement, loop extrusion, and transcriptionally mediated contacts. This specifically occurs as these 3 processes produce a small pocket of local density, the size of which depends on the strength of the process (e.g. many P-P interactions in a short distance would produce a larger number nucleosome-nucleosome juxtaposition). The local density distribution produced creates a gradient for enzyme penetration that depends on their size and the excluded volume produced within this region. If this local density gradient is sufficiently high, it results in preferential positioning of heterochromatin enzymes towards the interior and euchromatin enzymes near the outer zone and in the interdomain regions. Gene transcription reactions represent the most interesting case, since these reactions are non-monotonically dependent on local density (peak efficiency ˜0.2-0.35)(29, 47, 53, 54). As a result, transcription can both create domains and benefit from their formation from the resulting creation of an ideal functional ‘interface’ zone in mature packing domains.

One can visualize the model in the context of transcription as follows. Considering a genomic segment ˜100 kbp containing coding and non-coding regions spread out over a 100 cubic nanometer volume. Inefficient transcription reactions (P-P, E-P) and stochastic contacts create a pocket of increased local density consistent with a nascent domain (FIGS. 3a, 3b). If transcription remains ongoing, the density further increases within this pocket producing physical gradient of positions for heterochromatin and euchromatin enzymes due to their size (FIG. 3a). With respect to the polymeric assembly, the model predicts that domains will transition from a weakly assembled nascent domain (D˜2.2) to a stable-state domain (D˜2.8, FIGS. 3a-3c). In conjunction with domain maturation, transcriptional loops stabilize and persist at the domain periphery (FIGS. 3a-3d) with a resulting decrease in entropic loops.

The net integration of these processes is domain self-assembly with a mass-fractal geometry. Transcription initiates the conditions to create a domain, nucleosome-modifying enzymes mature the domains, and transcription benefits from the created zone while preventing further expansion. In the case of inhibition of RNA-transcription, two processes are predicted to occur from the non-monotonic dependence of transcription on local density. 1) A loss of nascent domains (FIGS. 3b-3d) and 2) unconstrained mature domain expansion as heterochromatin enzymes would proceed uninhibited outward. Reciprocally, active RNA-polymerase has an improved density for molecular activity, and from the model, it was expected that the loss of heterochromatin cores could impair transcriptional activity by the loss of its ideal physiochemical conditions (FIG. 3a). As such, even where a gene is accessible, the lack of improved conditions impairs transcription. This integration provides the basis for understanding the mechanistic role of chromatin packing domains, their formation, and function. Instead of heterochromatin and euchromatin being dichotomous partitions, in domain geometry they are an integrated physical and functional unit. Finally, this integration provides the mechanistic explanation of the paradoxically inhibition of transcription with inhibition of heterochromatin enzymes that is tested within the disclosure.

### Transcriptional Inhibition and RAD21 Depletion Result in the Loss of Nascent Domains

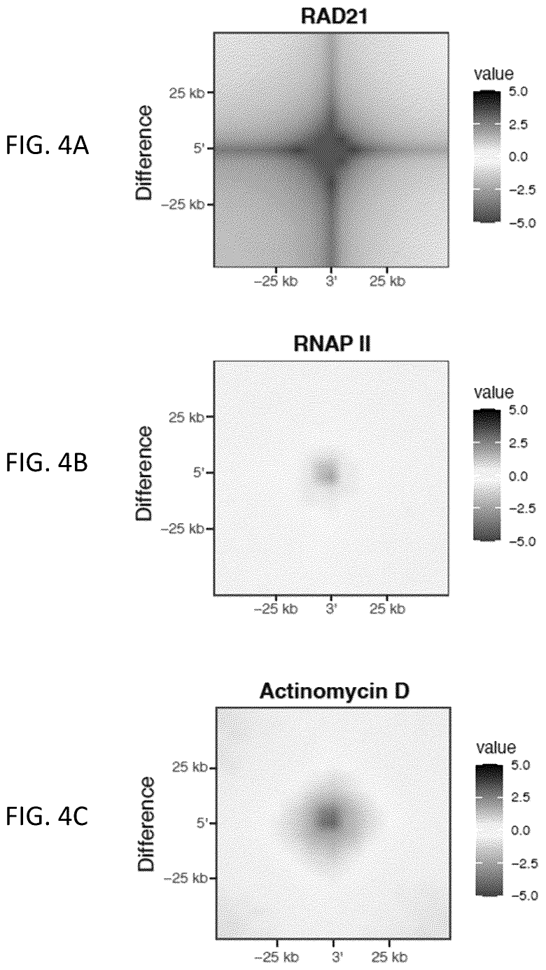

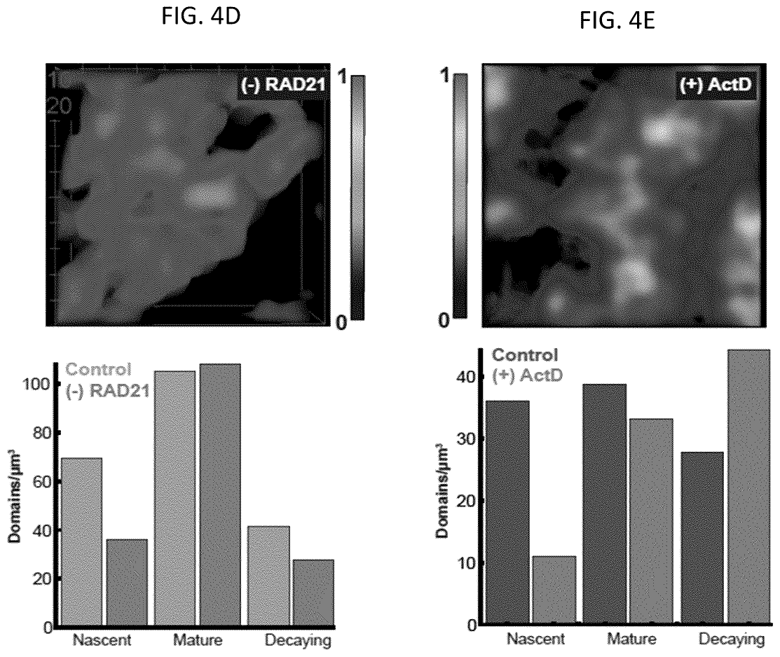

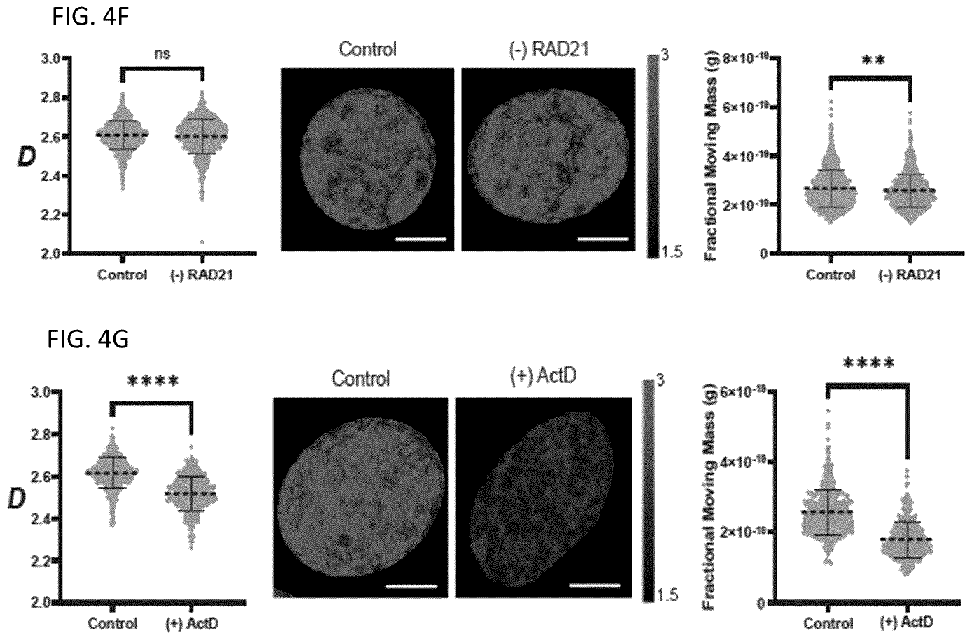

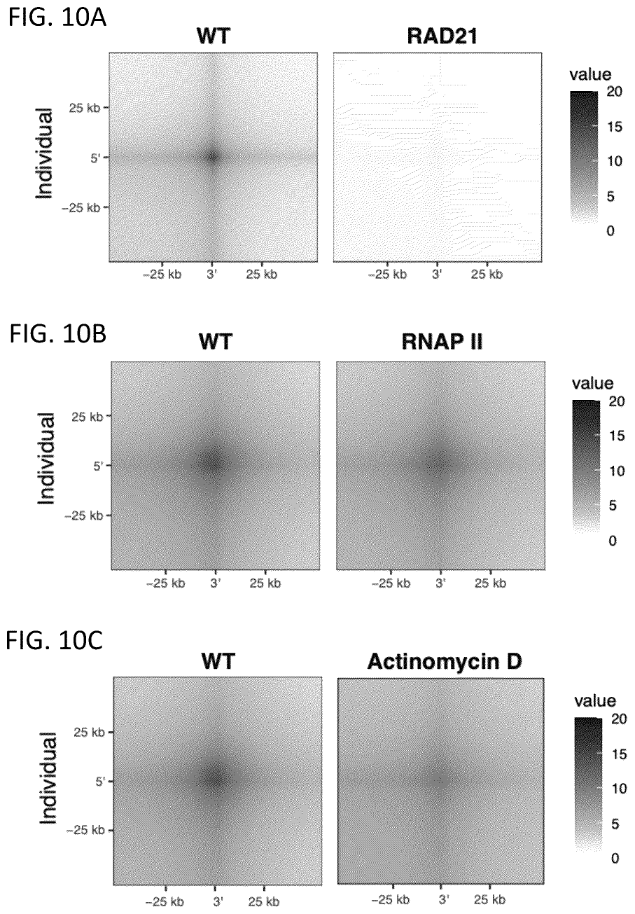

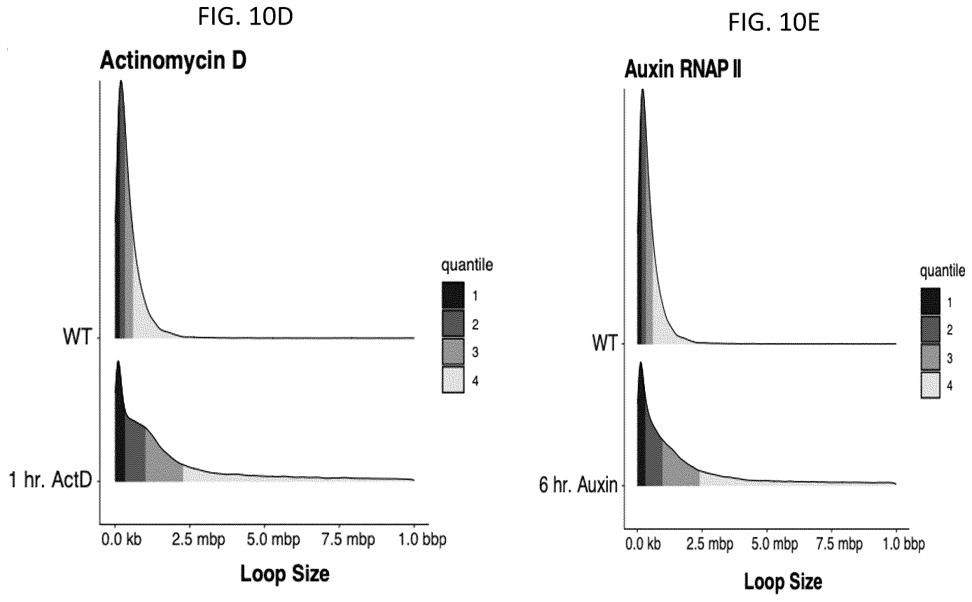

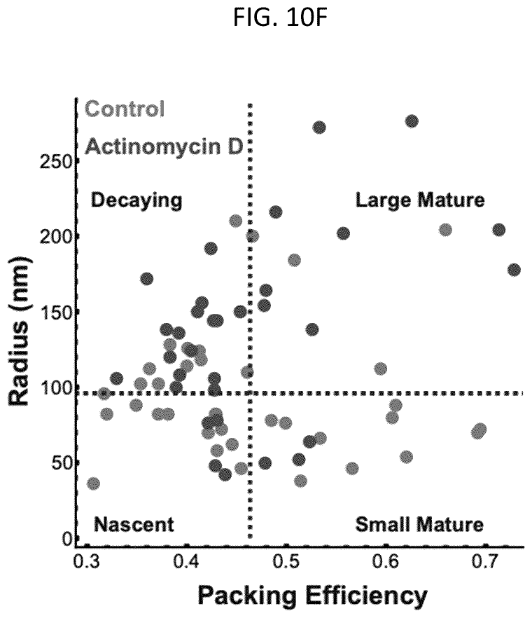

Next, the predictions of this model were tested at the level of nascent domain formation. As transcription and RAD21 loop extrusion produces long-range forced returns and interactions, the model predicts that transcriptional inhibition or RAD21 depletion results in primarily the loss of nascent domains. However, in contrast to RAD21, transcription would uniquely also act as a barrier element to prevent domain swelling by acting continuously along the intermediate zone of mature domains. To test this hypothesis, an RNA-polymerase II auxin-inducible degron-2 (AID2) and a RAD21 AID2 cell line were used, and Hi-C performed on cells with the depletion of these long-range regulators(58, 59). As expected, the frequency of loops decreased at sites where loops were initially found (FIGS. 4a-4b). To ensure the complete disruption of transcription, Actinomycin D (ActD) was then utilized to inhibit the activity of all polymerases within the nucleus(60). On Hi-C with 4 mm ActD treatment, decrease in loop frequency with transcriptional inhibition at the wild-type loci was again observed (FIG. 4c). Both RNA polymerase II depletion and ActD treatment results in an increase in entropic loops as observed in the formation of weak loop foci and the loss of native loops (FIG. 4b, FIG. 4c, FIGS. 10a-10f). To test the effect of this depletion on domain structure in situ, the change in domains observed by ChromSTEM tomography on cells treated with 4 mm ActD and on depletion of RAD21 was analyzed. Consistent with the predictions from the disclosed model, ActD treatment and RAD21 depletion both result in the loss of nascent domains, with a pronounced 69% decrease in nascent domains (FIGS. 4d-4e) upon ActD treatment.

Likewise, as predicted from transcription also acting as a barrier element to stabilize domains expansion, the swelling of a subset of domains into very large structures was observed (FIG. 4d) with low packing efficiency (60% increase) that was not observed on RAD21 depletion. To test if these predictions in domain organization extended into live-cells, live cell Partial Wave Spectroscopic (PWS) microscopy was performed to measure chromatin-average packing scaling Dₙ and the fractional moving mass of chromatin (FMM) in these conditions(29, 61, 62). While ChromSTEM measures the mass-fractal structure in individual domains, Dₙ measured by PWS microscopy is an ensemble average property of domains within a given chromatin region containing multiple domains and proportional to the D of individual domains and their volume fraction within the chromatin region. As long-range tethering would produce small clutches of nucleosomes moving as a paired functional element, a decrease in FMM would be consistent with impaired nascent domain assembly. Consistent with the findings on ChromSTEM tomography, a decrease in Dₙ and FMM on ActD treatment was observed consistent with the loss of domains and impaired formation of nascent domains. Further, ActD treatment on PWS microscopy results in a much larger decrease in Dₙ and FMM compared to RAD21 depletion from the loss of both nascent and mature domain structures resulting in an increase in decaying domains upon transcriptional inhibition (FIGS. 4f-4g). Notably, mature packing domains were maintained upon RAD21 depletion and decaying domains increased with ActD treatment, which were features not observed on Hi-C at the level of change in TADs in either condition.

Consequently, this indicated that packing domains are not merely a physical manifestation of TADs but represented an alternative regulatory framework in individual cells (FIG. 10f). This finding is consistent with prior work with SIM, showing that chromatin nanodomains defined by DNA staining and nucleosome modifications were found to be sub-TAD structures that were unaltered by the depletion of RAD21(24, 26). Due to the resolution limit of SIM(80-100 nm), the challenge of converting the observed SIM signal intensity directly into mass density and the challenge of mapping SIM images into polymeric folding, it would be difficult to probe the life-cycle of domains observed on ChromSTEM exclusively with this modality(24, 26).

### Nucleosome Modifications and Transcription Regulate Domain Stability

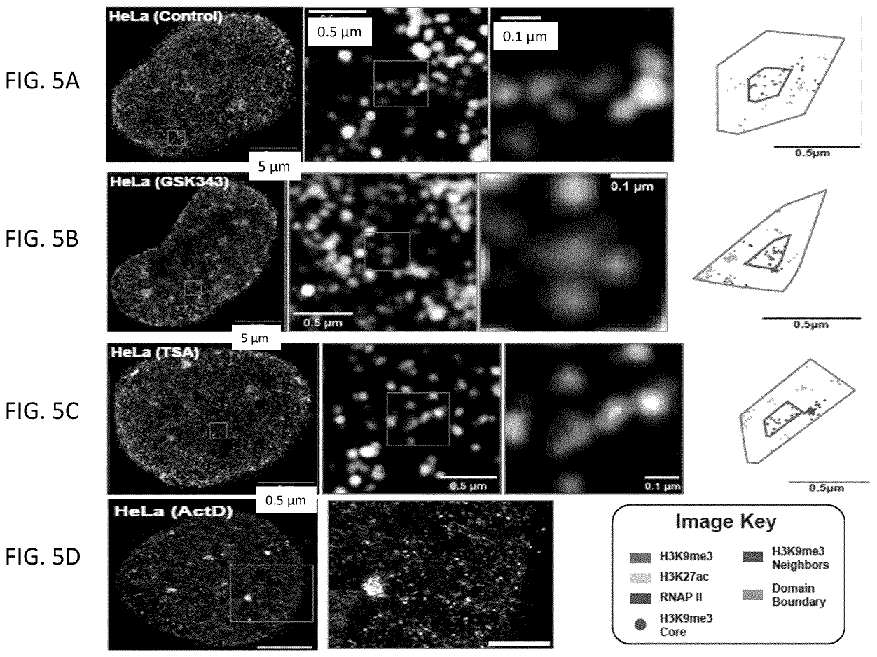

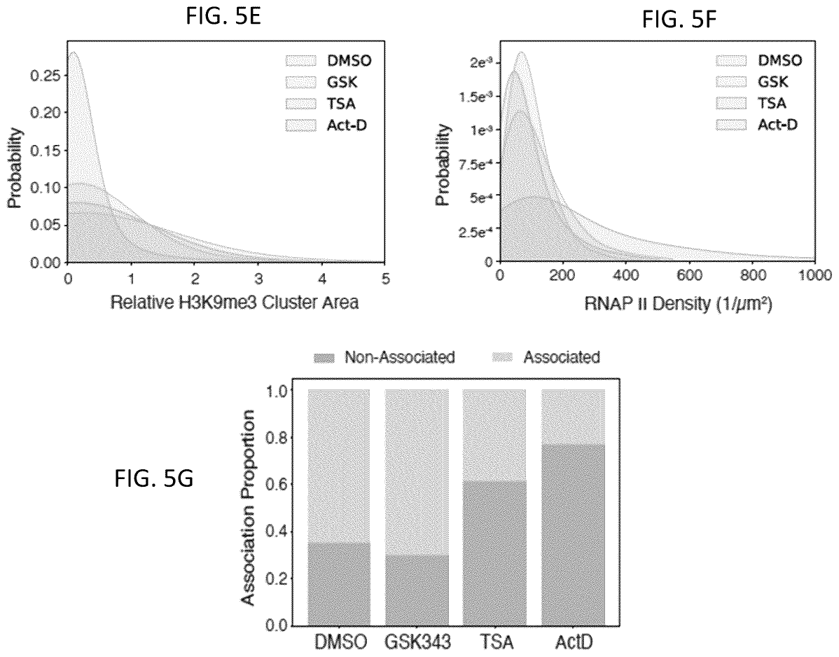

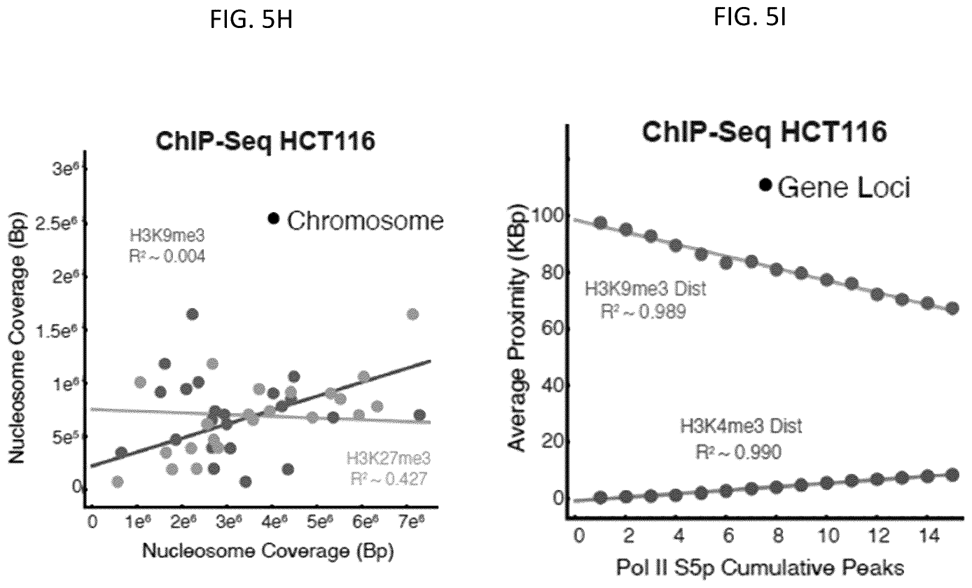

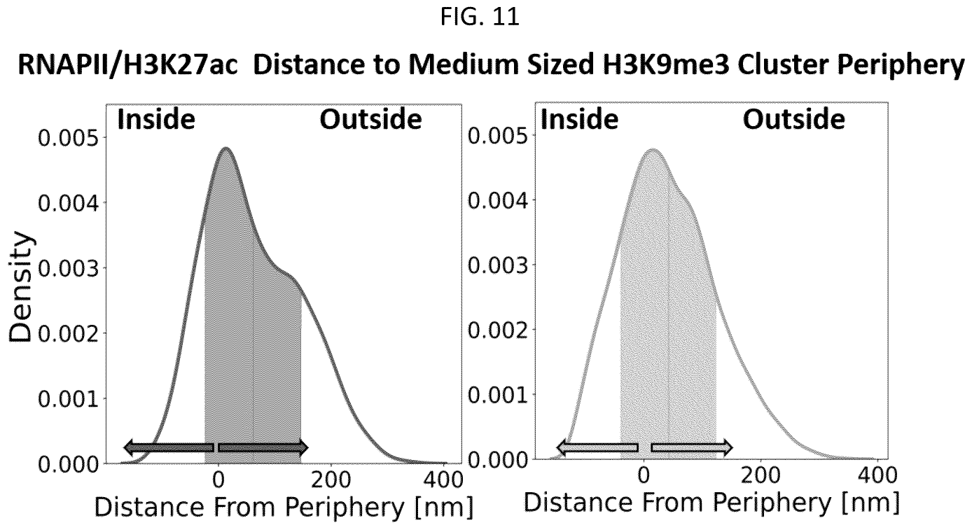

As evidenced from the model and ChromSTEM tomography, the observed PDs may align with the various structures observed by SIM and SMLM if heterochromatin is observed to spatially associate with active RNA polymerase and euchromatin modifications as a unified, folding geometric structure described above (24, 26, 28, 34, 63, 64). For these structures to be related, the distance at which polymerase and euchromatin marks occur at the boundary markers of a well-formed (presumably constitutive) heterochromatin core due to this region corresponds to the densities (˜3-5× the area of the dense interior) for the euchromatic and transcriptional enzymatic complexes (FIG. 1f, FIG. 11). Although improved transcriptional efficiency occurs for polymerases associated with dense domains, transcription must still occur (albeit less efficiently) in areas not associated with mature domains; otherwise nascent domain formation would be rare. As such, it would be expected that a proportion of active RNA Pol-II is decoupled from constitutive heterochromatin and associate either with facultative heterochromatin or proto-domains (FIGS. 1-3). While not explored within this work, this consideration of the distribution of domain sizes, densities, and polymerase activity could have functional consequences depending on more complex cell states (e.g. differentiation, senescence, stem cells, and dormancy); each of which could have an associated polymerase-to-domain landscape. To test this hypothesis of frequent but not exclusive colocalization, 2 and 3-color SMLM of serine-2 phosphorylation of Pol-II (Pol II-Ps2, associated with elongation)(65), with H3K27 acetylation (euchromatin), and H3K9me3 (constitutive heterochromatin was performed (FIG. 5a). To test if heterochromatin cores and active Pol-II are codependent: (1) EZH2 (GSK343, FIG. 5b)(66) was inhibited to disrupt nascent domain maturation (H3K27me3); (2) histone deacetylases (HDACs) were inhibited with trichostatin A (TSA)(57) to disrupt existing mature heterochromatin domains (FIG. 5c), and (3) transcription was disrupted with ActD (FIG. 5d).

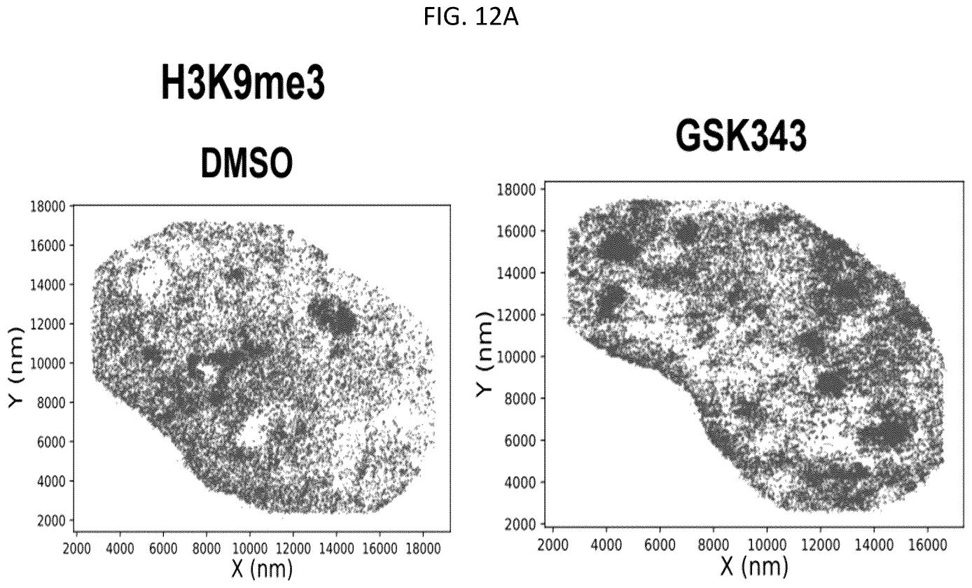

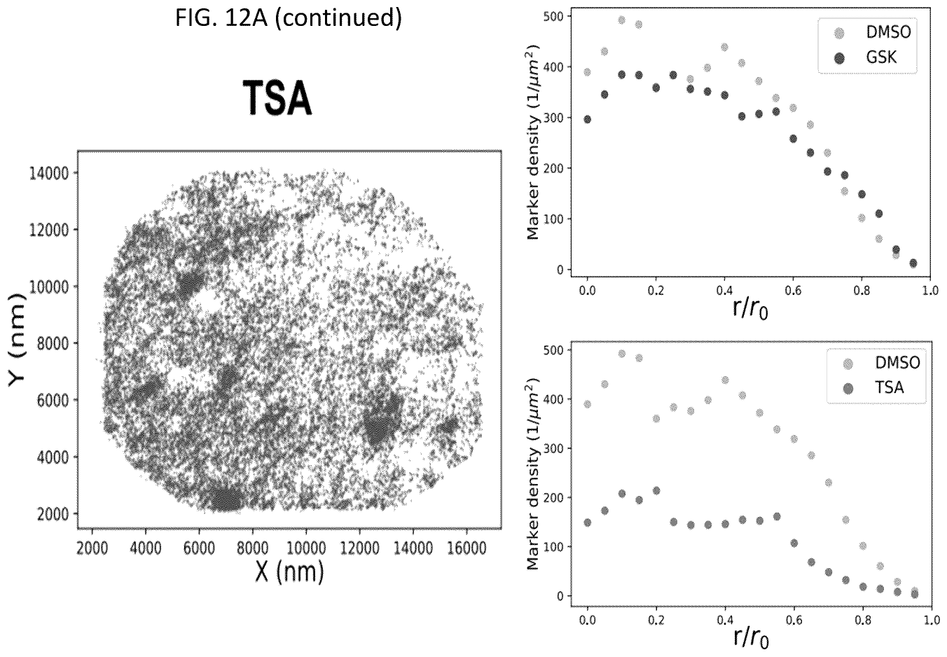

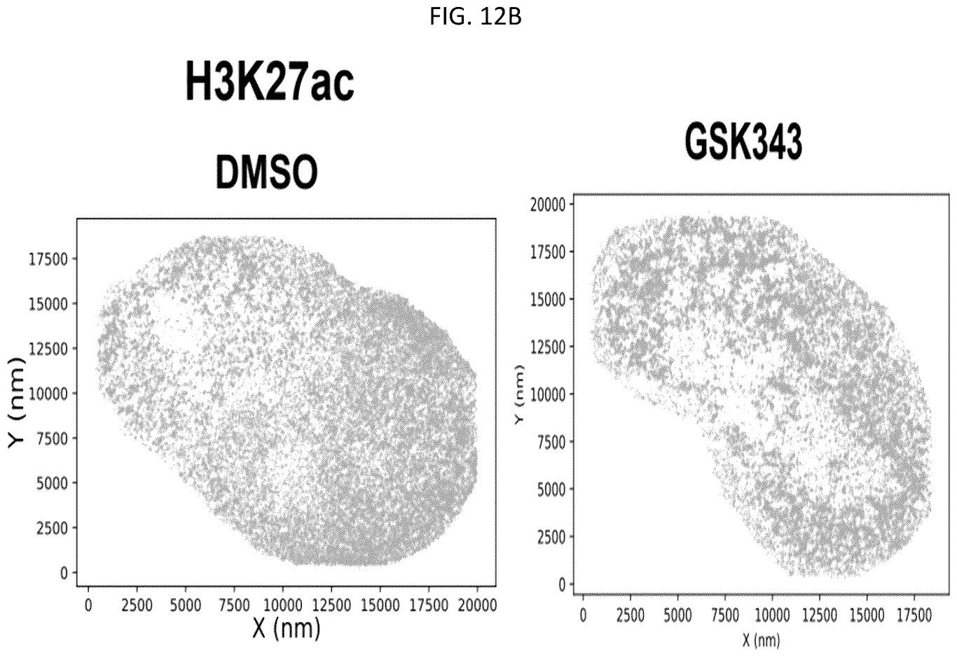

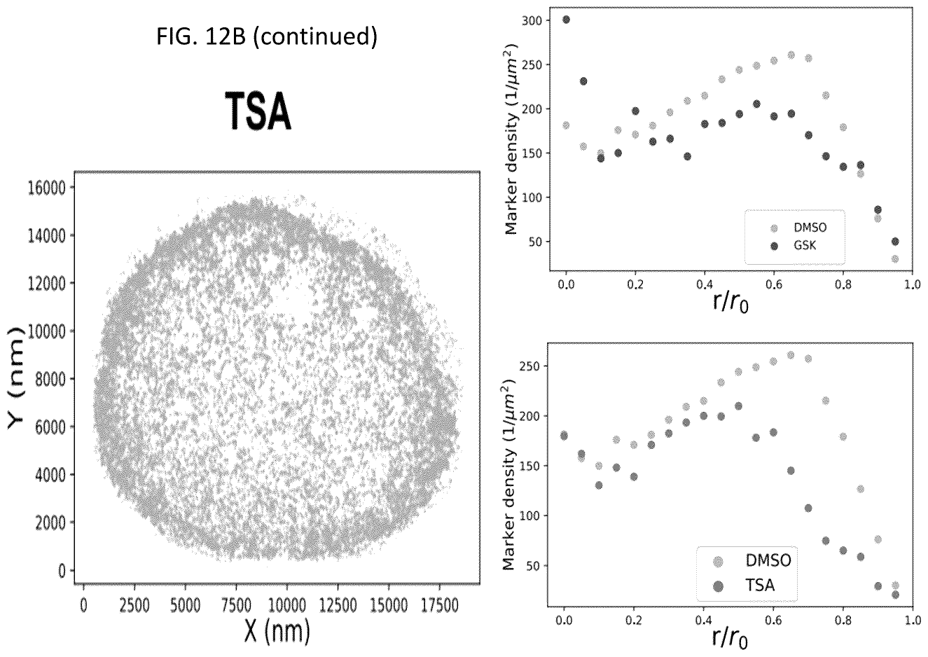

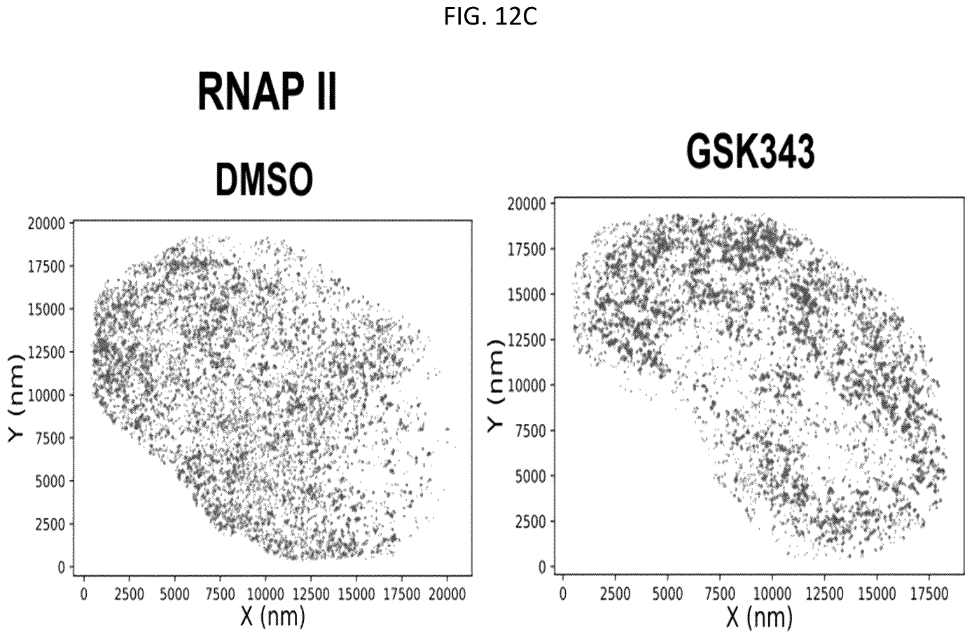

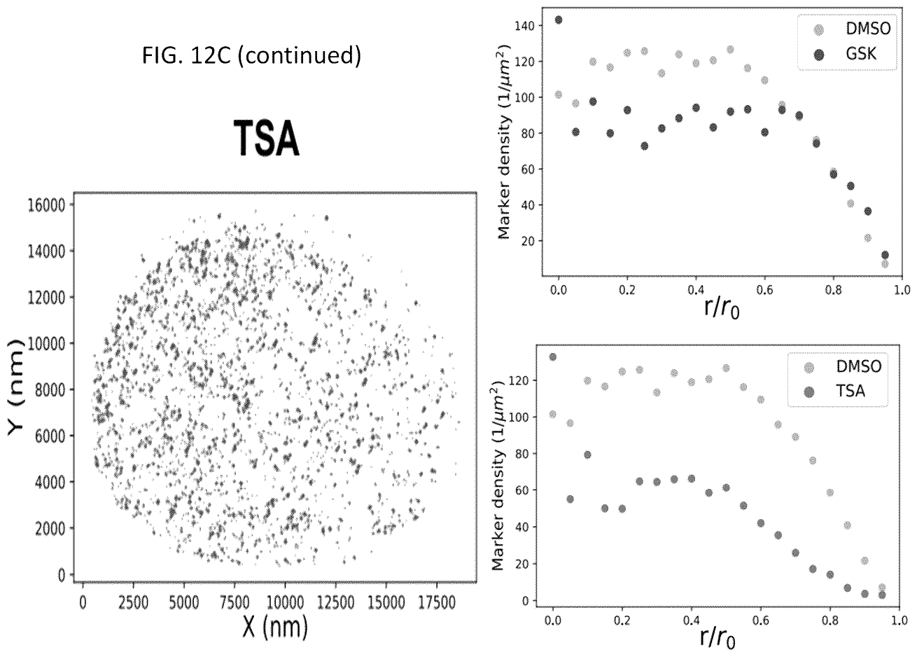

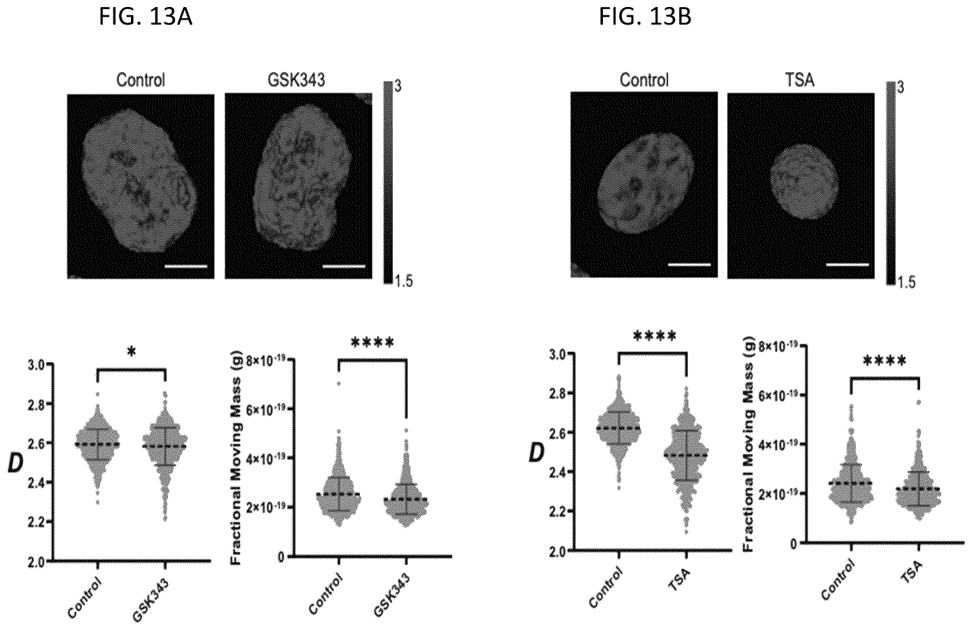

From the model, it is predicted that H3K27me3 depletion would result in the partial loss of H3K9me3 cores indirectly due to the disruption of domain maturation process, with the primary decrease in active Pol-II Ps2 loci occurring outside of H3K9me3 domains (a prediction that contrasts with a model of domains as continuously transitioning in density from H3K4me3 to H3K27me3 surrounding H3K9me3)(24). HDAC inhibition, with the direct disruption of mature domains, would produce several features (1) the depletion of H3K9me3 would result in the loss of H3K27ac and Pol-II Ps2 predominantly within the low-density nuclear interior, (2) there will be a greater loss in RNA polymerase loci in comparison to EZH2 inhibition due to the loss of optimal transcriptional domains, and (3) the remaining Pol-II Ps2 would be mainly decoupled from H3K9me3. In strong agreement with the disclosed model, these specific predictions were indeed observed (FIGS. 5e-g), including the previously unexplainable central elimination of both H3K27ac and H3K9me3 associated with TSA treatment on microscopy (FIGS. 5c, 5e, 5f; FIGS. 12a-12c). ActD mediated transcriptional inhibition resulted in overall decrease in domain number with swelling of the remaining H3K9me3 domains as observed on ChromSTEM (FIG. 4) and also resulted in decoupling between active Pol-II and heterochromatin domains (FIGS. 5d-g) consistent with RNA polymerase facilitating domain assembly and acting as a barrier element to prevent domain expansion. Extended further to live cell nanoscopy, the inhibition of transcription, the impairment of domain formation from EZH2 inhibition with GSK343, and the disruption of mature domains with HDACi via TSA all result in loss of domains as evidenced by a decrease in Dₙ and the impaired ability for domain formation as measured by a decreased FMM(FIG. 13).

Consistent with the findings on ChromSTEM and in the disclosed model, we found both the dissolution of constitutive H3K9me3 as well as the decoupling of Pol-II Ps2 from these structures (FIGS. 5a-g) with the inhibition of heterochromatin and transcription. If this spatial coupling extended into population data of loci for specific genes, it was expected that similar features would be observed on chromatin-immunoprecipitation sequencing (ChIP-Seq) analysis by investigating the distal behavior of these features in 1D. With its role as a scaffold for nascent domain maturation, it was predicted that H3K27me3 levels would correlate with the level of transcription on a long-range basis (e.g. on a per-chromosome analysis) whereas H3K9me3 would primarily couple with polymerase locally. As such, at the kilobase pair to megabase pair range, the model predicts that as RNA polymerase II density increases, the distance to H3K9me3 will decrease due to the increased efficiency of packing from transcriptional reactions. H3K27me3 will in contrast grow further away from genes with increasing level of Pol-II due to the transformation of domains from immature to mature structures. Using publicly available ChIP-Seq data of H3K27me3, H3K9me3, and initiated polymerase (Pol-II Ps5) available through ENCODE(67-69), these predictions hold with a correlation between H3K27me3 and polymerase coverage per chromosome (FIG. 5h). Likewise, as the density of Pol-II Ps2 per gene increases, the distance to the nearest H3K9me3 decreases monotonically (FIG. 5i) from ˜100 Kbp to ˜60 Kbp on average(65).

### Divalent Ions and Domain Stabilization.

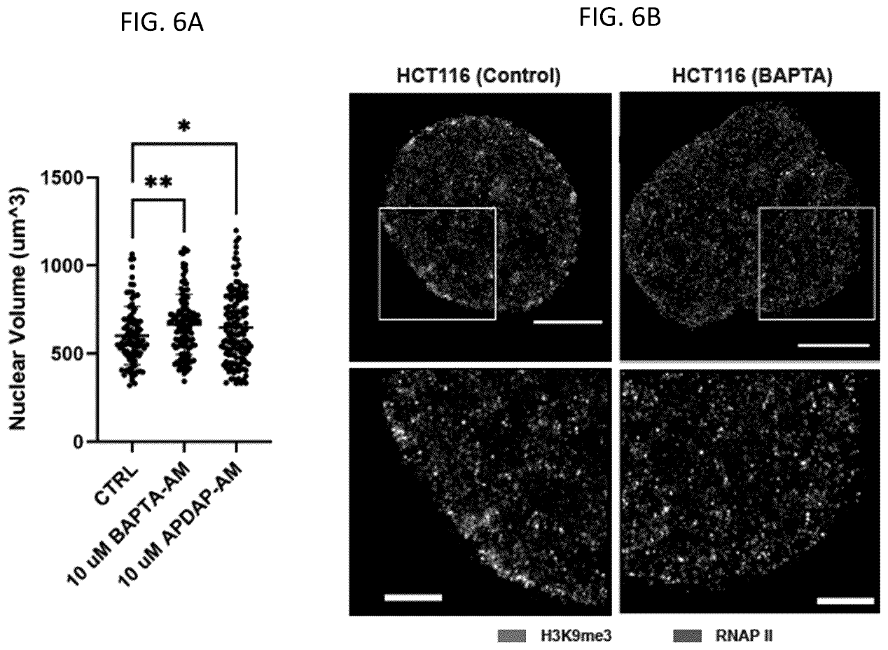

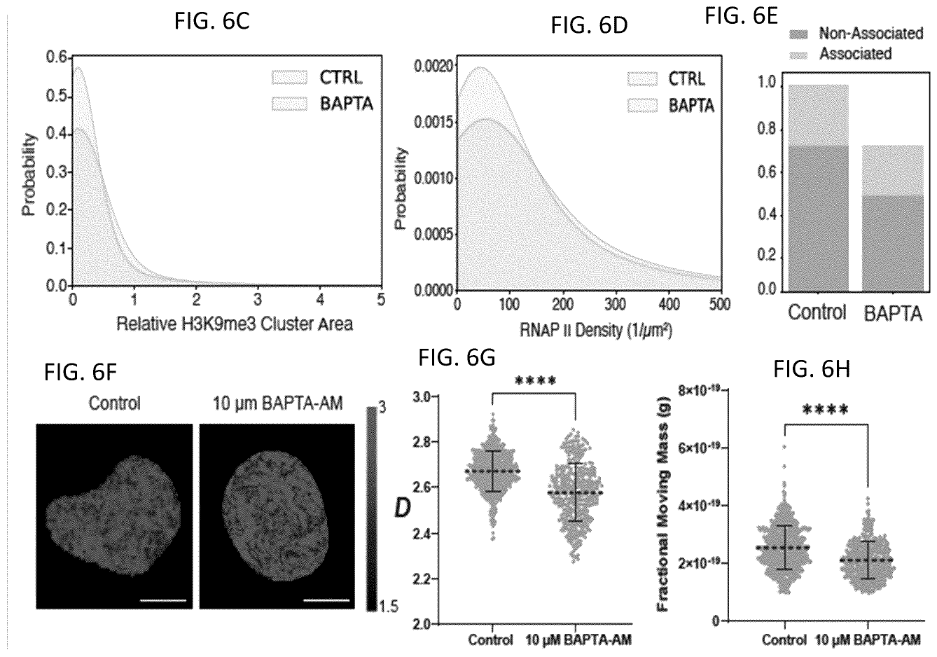

Given the large accumulation of charged polyphosphates within the limited space of the domain interior, it was expected that counter-ions, especially multivalent ions such as magnesium and calcium, would be helpful to maintain domain integrity and nuclear size. In particular, the efficient conversion of nascent domains into mature structures will depend on the local density of DNA (and therefore nuclear volume) and as a result will use substantial charge neutralization to produce domain stability. Consequently, the loss of divalent counter ions could prevent domain maturation and swell mature domains resulting in a loss in the total number of mature domains with increased size of remaining heterochromatic cores while also abating optimal conditions for transcription. On SMLM this would be consistent with the decrease in total number of H3K9me3 cores, an increase in the size of the remaining structures, and a decrease in the Pol-II Ps2 density surrounding the remaining domains. To test this hypothesis, targeted chelation of intracellular divalent cations (Ca²⁺, Mg²⁺) with BAPTA-AM, and multicolor SMLM were performed. On confocal microscopy, nuclear volume increases as expected from chelation of divalent ions (FIG. 6a). Consistent with the hypothesis that domains depend on ionic regulation, the number of domains decreased upon chelation (FIGS. 6b, 6f), with increased size of the H3K9me3 clusters remaining, and a decreased density of Pol-II Ps2 surrounding each cluster (FIGS. 6b-6e). To directly investigate the influence of divalent cations on chromatin organization in living cells, chromatin conformation was measured from the level of packing domains using PWS microscopy. Consistent with the results on SMLM upon BAPTA chelation, a decrease in nuclear Dₙ and FMM was observed, indicating a loss in mature domains and the process of their formation due to divalent cation chelation (FIG. 6f) comparable in magnitude to the effects of TSA-mediated HDAC inhibition (FIG. 13).

### Inhibition of Heterochromatin can Suppress Transcription In Situ

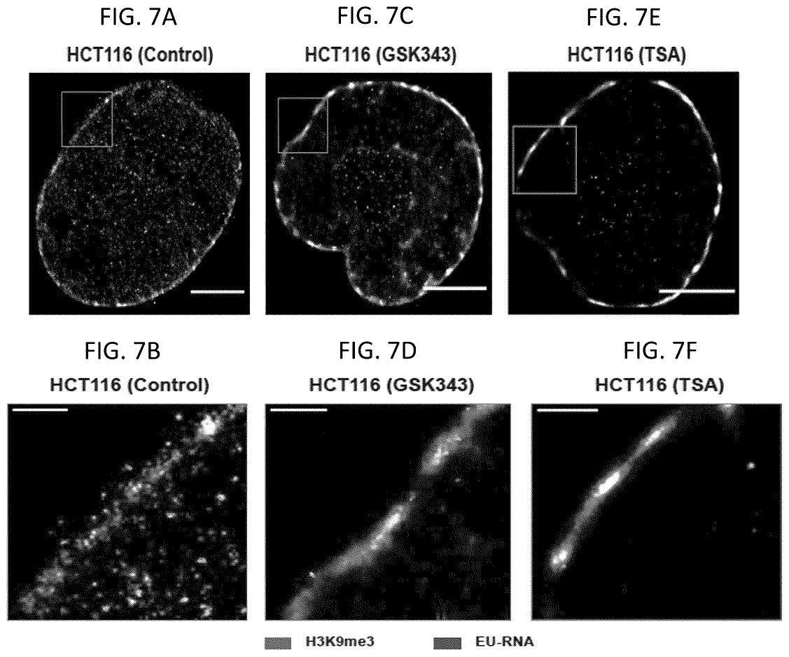

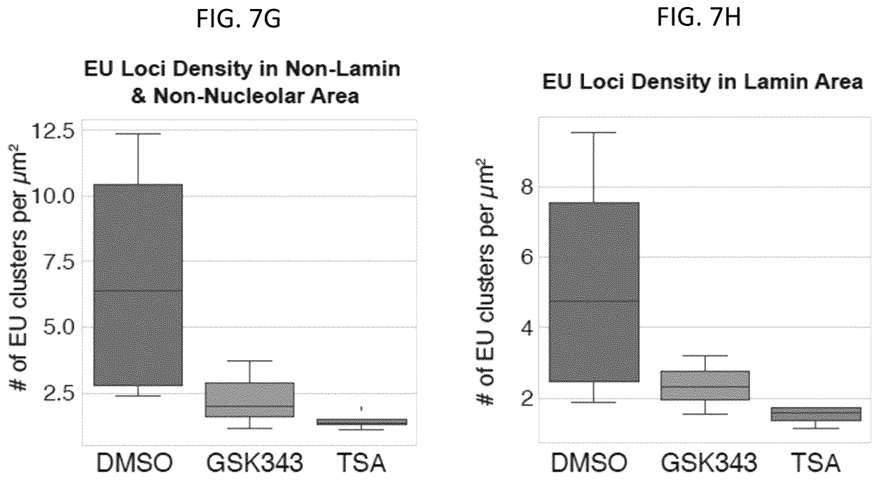

From the model and the experimental results so far, an unexpected prediction is that heterochromatin formation is important for proper transcription to occur (FIG. 3, FIG. 4, and FIG. 5). Further, the efficiency of heterochromatin formation will depend on nuclear chromatin volume concentration (CVC) due to the effect volume has on enzyme distributions; therefore decreased heterochromatin enzymatic activity or an increase in nuclear volume could result in a paradoxical global transcriptional decrease. This process arises from the non-monotonic dependence of RNA polymerase on local crowding conditions in addition to its dependence on molecular features such as the dissociation constant for specific loci, TF concentrations, and TF-sequence binding affinities(29, 47, 53). Consequently, an overly accessible genomic segment without optimal physical conditions would be expected to have inefficient RNA synthesis. To test this prediction, HCT-116 cells were used, whose average CVC was observed to be ˜0.2, and inhibited EZH2 with GSK343 and HDAC with TSA as above. Given the initial CVC, these perturbations would be expected to paradoxically disrupt transcription globally. Using 2-color SMLM pairing H3K9me3 structure with nascent RNA synthesis via EU staining(63) in HCT-116 cells for 1 hour, the resulting change in RNA synthesis with heterochromatin disruption was measured. In DMSO control HCT-116 nuclei, there is the visually apparent association between nascent RNA and H3K9me3, with a distribution of events observed both near the nuclear periphery and further into the interior (FIGS. 7a, 7b). With GSK343-mediated inhibition of EZH2, a striking loss of EU signal is observed visually (FIGS. 7c, 7d) within the non-nucleolar chromatin. As with EZH2 mediated inhibition, TSA treatment produces a marked decrease in the concentration of EU clusters (FIG. 7e, 7f). On spatial analysis, within the non-nucleolar interior it was observed that mRNA synthesis over an hour burst produces ˜7.5 clusters per mm² (FIG. 7g). Quantitively, a decrease in EU foci to 2 clusters per mm² with GSK343 treatment was observed, representing a 60%, and a drop in EU concentration to 1 cluster per mm² in the non-nucleolar chromatin, representing an ˜80% decrease across the whole nucleus with TSA treatment. Near the nuclear border, an initial lower-rate of RNA synthesis was observed as expected, but a similar decrease in the total amount of RNA synthesis across GSK343 and TSA treatment (FIG. 7h). In the context of similar studies demonstrating a paradoxical dependence of RNA synthesis on HDAC function, these results indicates how the disruption of domain geometry can result in the inhibition of gene transcription even as accessibility increases throughout the nucleus(17-19).

### H3K9Me3 Domain Cores Form in Non-Coding Regions of Terminal Myogenic Genes During Differentiation and Depend on Nuclear Volume

Since CVC and heterochromatin are codependent in facilitating RNA synthesis from the perspective of chromatin domains, it was expected that aging-associated nuclear swelling(70, 71) with the resulting loss in heterochromatin(70, 72, 73) could be associated with the transformation in chromatin PDs from the disruption of the self-assembly process. As such, the increased accessibility of the genome in aging can, in addition to the de-repression of stem genes and promotion of increased DNA damage, also result in anergic transcription of necessary genomic locations. At the other end of the developmental spectrum, it is thought that heterochromatin accumulation represses inappropriate lineage-genes to facilitate terminal differentiation(74). A model of these extremes is myogenesis and sarcopenia in aging. Clinically, understanding the regulation of muscle homeostasis has broad implications in human disease as sarcopenia is an independent prognostic marker of all-cause mortality and associated with impaired quality of life(75-77). Likewise, myogenic differentiation is a universal transcriptional network applicable to all vertebrate animals, with distinct roles of myogenic regulatory factors in the induction of stem cells into myoblasts and terminal differentiation as myotubes and muscle fibers(78, 79). With these considerations in mind, myogenic differentiation and nuclear-associated swelling in sarcopenia were investigated as applicable testbeds for the predictive power of the model relevant to development and aging. As demonstrated so far, heterochromatin formation within packing domains ensures proper domain geometry for transcription to occur on the periphery of mature domains. Consequently, the formation of H3K9me3 in noncoding segments would be hypothesized to associate with transcriptional activation by the self-assembly process. In terminal muscle differentiation, myogenin (Myog) is a transcription factor regulating the transition from immature myoblasts into myotubes with its targeted deletion in mice resulting in complete loss of mature skeletal muscle(80, 81). A key target of Myog are the myosin heavy chain genes, which are the primary structural component of skeletal muscle for appropriate mechanical contractility. As such, for terminal differentiation of myoblasts to myotubes to occur, Myog and myosin heavy chain genes are upregulated. From the three physical rules of the model: (1) long range interactions (e.g. a loop) should be evident prior to differentiation, (2) H3K9me3 will accumulate adjacent within a noncoding region, and (3) transcription amplifies in the formed domain with ideal configurations.

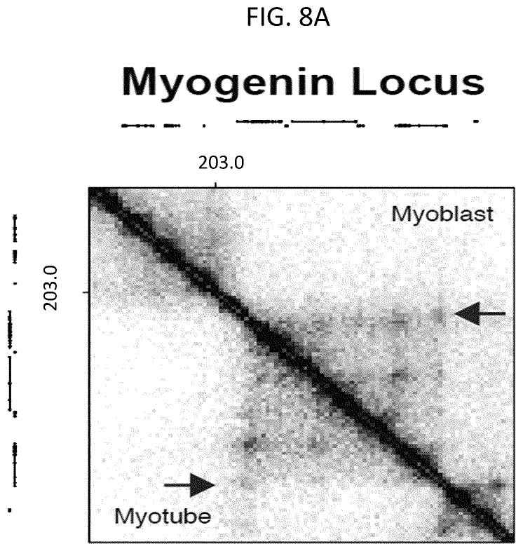

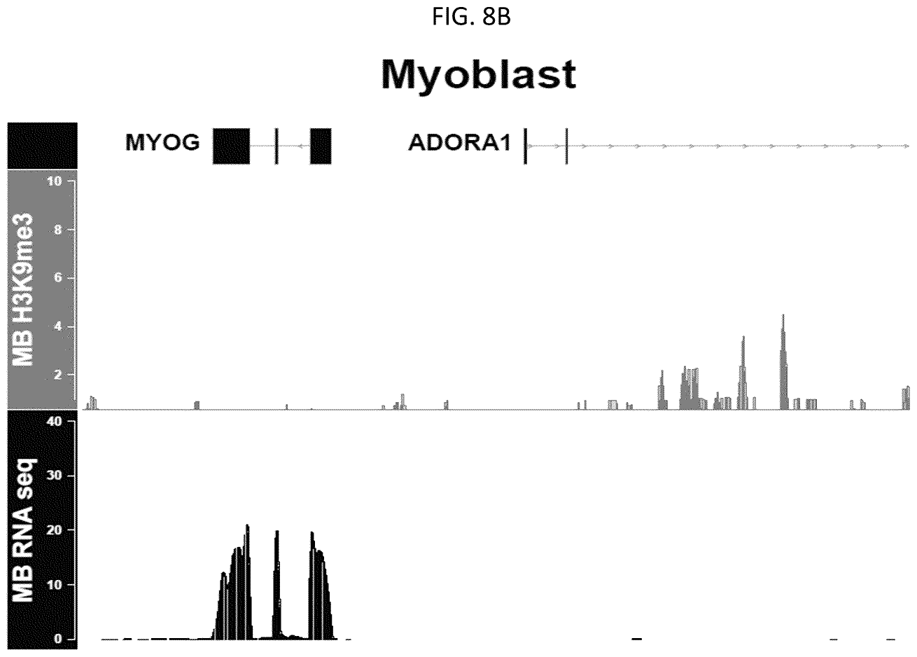

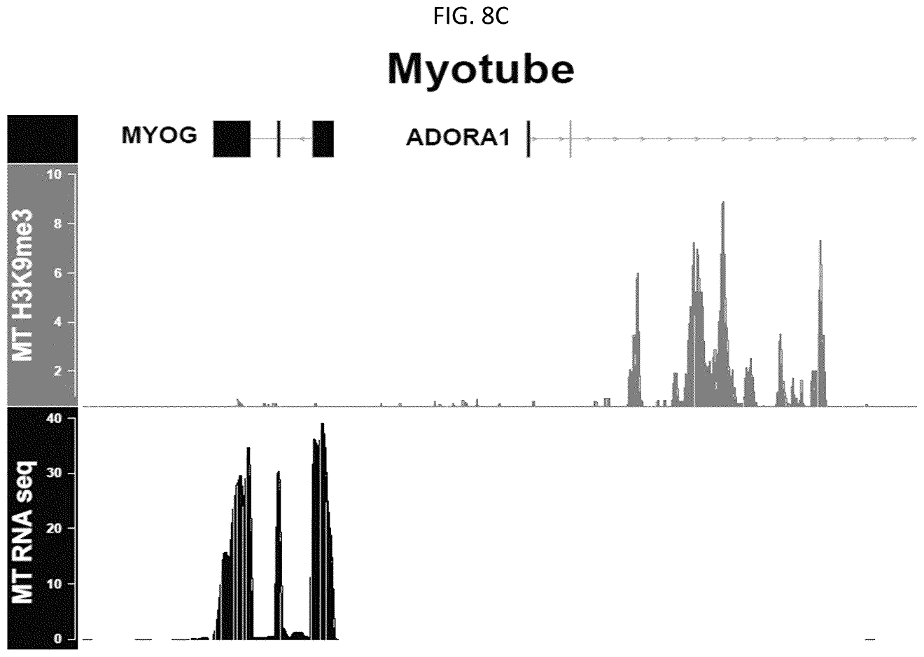

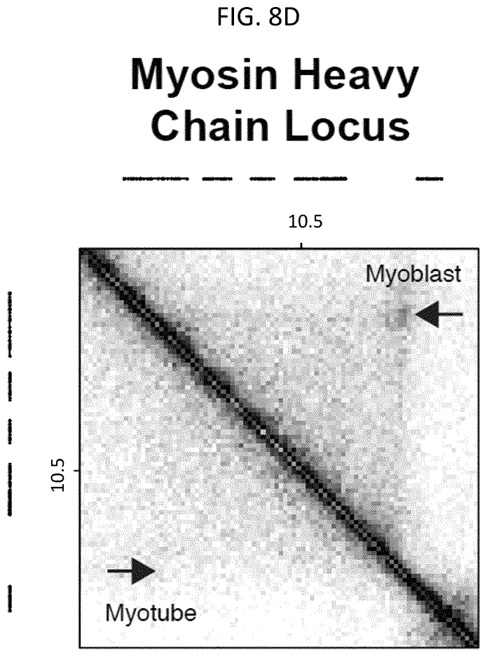

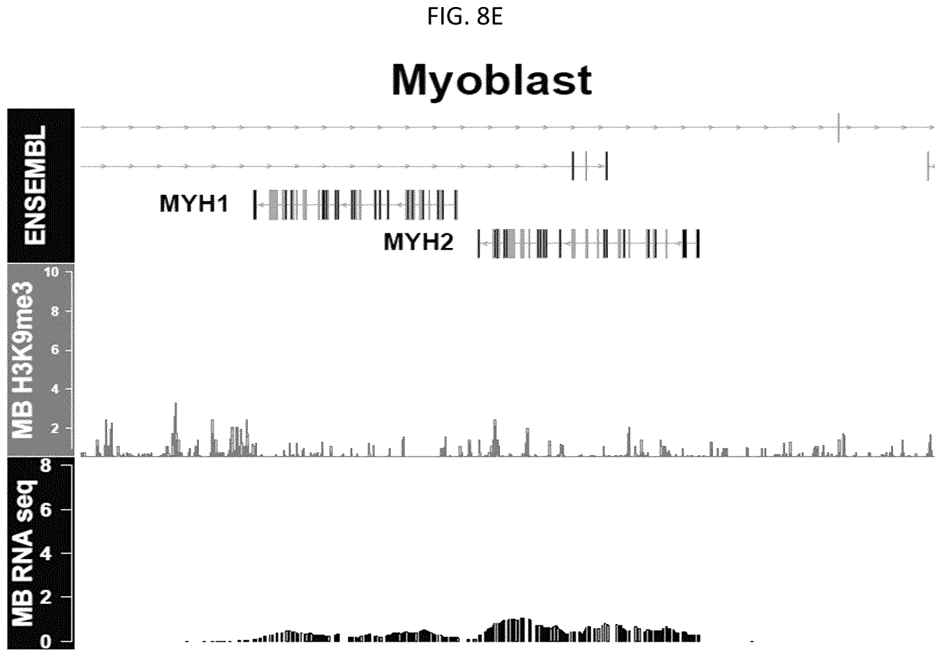

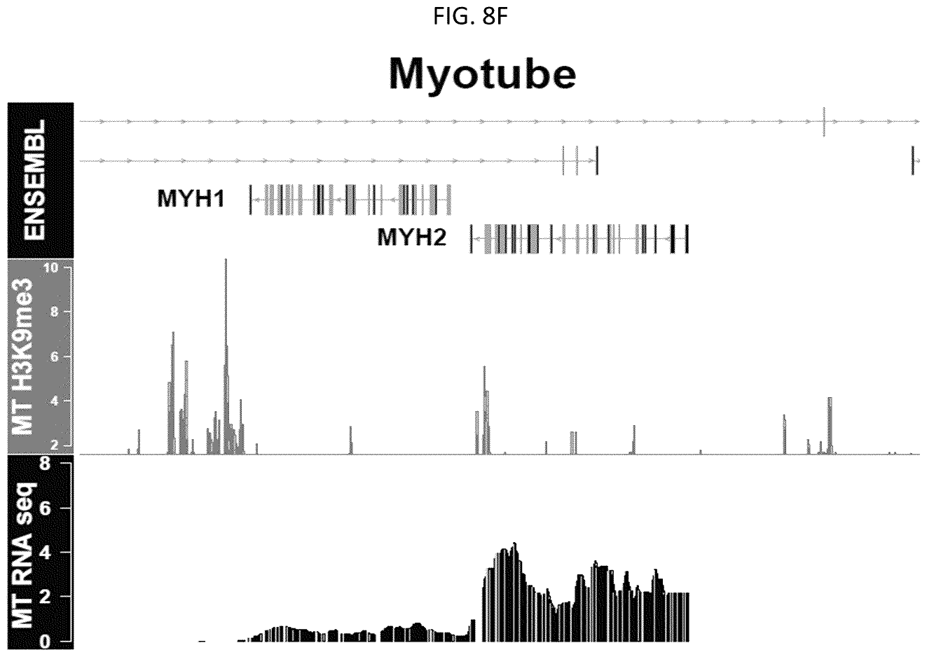

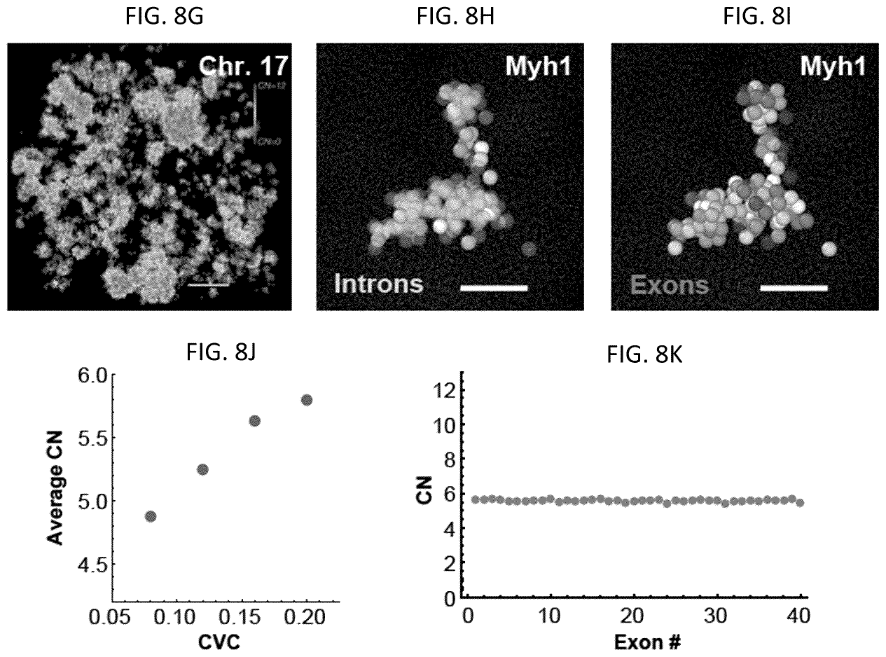

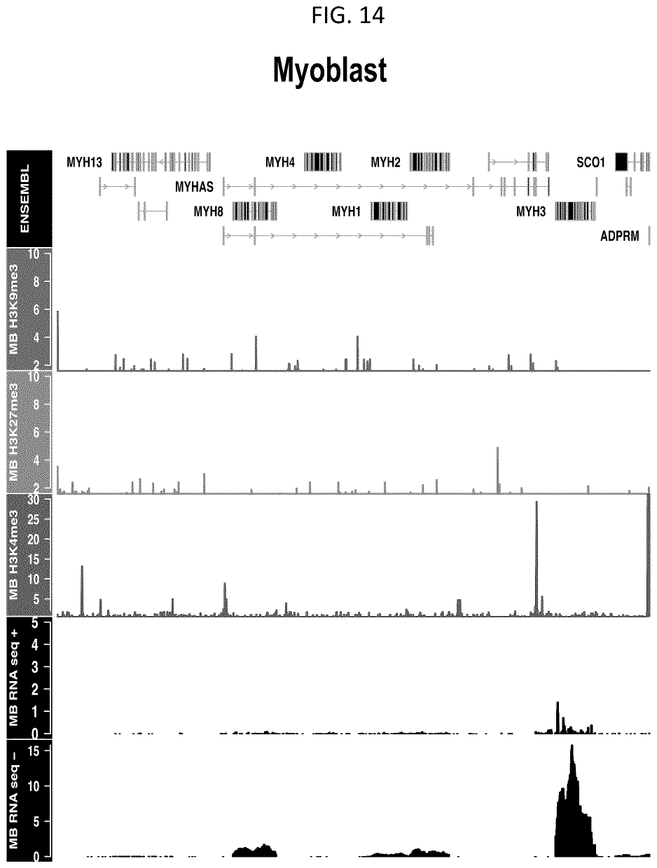

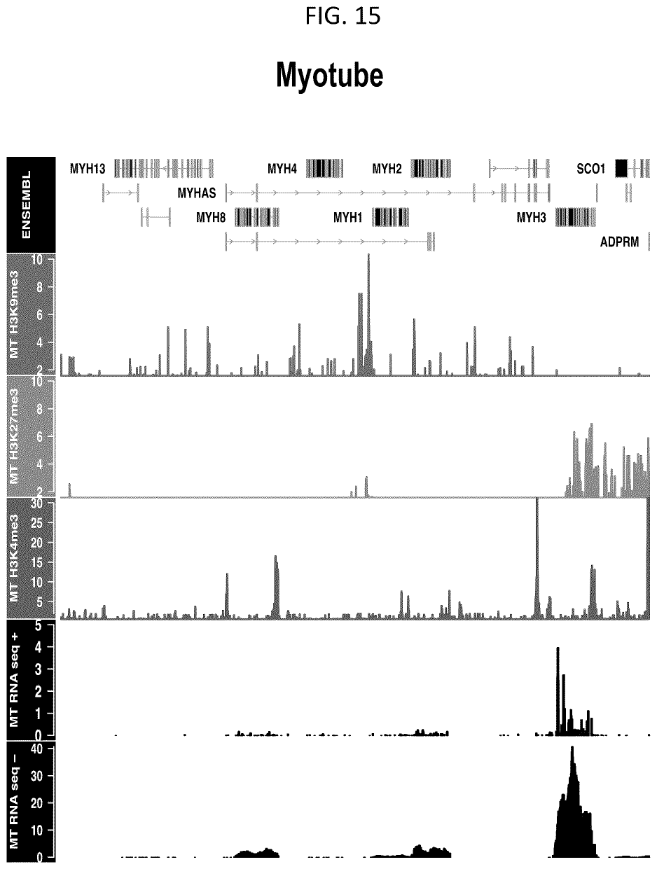

To test this hypothesis, publicly available ChIP-Seq data through the ENCODE consortium(67-69) of H3K9me3, H3K27me3, H3K4me3, and gene expression were utilized and paired with Hi-C of myoblasts and myotubes to examine the process of domain maturation. As expected from the disclosed model, a visually apparent chromatin loop domain dissipates and H3K9me3 accumulates adjacent to myogenin (FIGS. 8a-c) from the transition from myoblasts into myotubes. With respect to the myosin heavy chain loci, the depletion of an adjacent chromatin loop (FIG. 8d) with the accumulation of H3K9me3 in noncoding regions adjacent to myosin heavy chains 1, 2, 4, and 8 was again observed (FIG. 8e, FIGS. 14-15). Indeed, accumulation adjacent to the fast-twitch myosin heavy chains (1 &2) that are associated with adult skeletal muscle function were predominantly observed (FIG. 8f, FIG. 15).

Naturally, sarcopenia would present the inverse of this process at these loci if disruption in chromatin packing domains occurs from aging in muscle cells. To model this process, the effect of nuclear swelling and its result on domain structure were considered from the considerations presented above. In contrast to topological features such as TADs and A/B compartments, packing domains are impacted by nuclear swelling due to rules two and three(45). The focus was on myosin heavy chain 1 (Myh1) as its translated product decreases in aged muscle and therefore may represent sarcopenic transformation(82). Using SR-EV, configurations of chromosome 17 with a fixed probability of long-range returns (alpha=1.15) were generated, and the effect of decrease CVC from 0.2 to 0.08 on the structure of the domain cores at Myh1 investigated (FIGS. 8G-8I). By calculating the coordination number (CN) from SR-EV, the likelihood of a gene segment being within a high-density region (CN>8), ideal region (6-7), and outer zone region (CN<5) could be estimated. Directly from the change in volume, a decrease was observed in the localization of exon elements from ideal conditions (˜contact with 6 nucleosomes) toward accessible but less ideal configuration (contact with ˜4-5 nucleosomes) as nuclear volume increased (FIGS. 8h-j). Unexpectedly and consistent with the model, the exon segments of Myh1 with a CVC of 0.16 generated with SR-EV were on average localized to ideal transcriptional conditions (FIG. 8k). Consequently, domain assembly was both observed at gene locations for muscle differentiation and the transformation of domains could occur from a process such as aging-associated nuclear swelling. In sum, these findings were consistent with a role for domain assembly and maturation, with their degradation providing an additional deleterious consequence of pathological nuclear swelling.

### Discussion

In this work, the structure of chromatin packing domains identified on ChromSTEM tomography (FIG. 1) and their function in regulating gene transcription (FIGS. 2-4) were investigated. Packing domains are heterogeneous, conformationally defined assemblies whose function is tightly integrated with their mass-fractal geometry (FIG. 1f, and FIGS. 4d-g). These domains exist across a conformational life cycle-nascent (poorly packed, small) domains form during transcription and loop extrusion, mature (efficiently packed) domains provide an ideal physical scaffold for transcription, and finally, these domains collapse into decaying structures (large, poorly packed). This progression bidirectionally links chromatin structure with gene regulation, as each stage of the life cycle reflects functional shifts in transcriptional activity (FIGS. 3, 4, and 8).

The disclosed modeling and simulations, coupled with experimental observations from ChromSTEM, multi-color SMLM, and PWS nanoscopy, indicate that nanoscopic chromatin organizations observed by other groups, including nucleosome clutches and heterochromatin domains/nanodomains, are part of the described domain-forming system(24, 26, 28, 33, 34, 63). Importantly, these collective findings demonstrate that connectivity does not necessarily translate into 3-D space filling conformations. Chromatin topology, as an ensemble property of a large number of cells, is not congruent to chromatin conformation. Packing domains are not simply the physical manifestation of TADs. Unlike TADs, which arise in measurements of connectivity, packing domains undergo continuous structural transformations, driven by transcription and chromatin remodeling (FIG. 4). As shown, cohesin (enzyme that generates TADs) functions through generation of loop connections, creating nascent domains. Mature domains persist after RAD21 depletion, indicating that the regulatory function of mature domains is then independent of loop extrusion, including from ionic conditions and heterochromatin enzymes (FIGS. 5-7). Additionally, the formation of nascent domains is separately driven by transcription itself, and disruptions in domain maturation inhibit mRNA synthesis, illustrating the role of chromatin packing in transcription (FIGS. 2, 3, and 8).

The interplay between transcription, heterochromatin remodeling enzymes, ion concentrations, and nuclear density suggests a complex regulatory system that bidirectionally integrates gene expression with chromatin organization. It is these properties that indicate packing domains and transcription are coupled as emergence phenomenon: the intersection of three-rules creates a complex regulatory, self-evolving structure. First, these findings demonstrate that packing domains are self-assembling structures, forming through conformationally defined processes. Second, transcription depends on the deposition of heterochromatin in non-coding regions to maximize the efficiency of gene expression (FIG. 3). Finally, this model explains why disruptions in heterochromatin, as seen in nuclear swelling during muscle aging(72, 82), result in impaired cell function (FIG. 8). Insufficient nuclear density for domain maturation leads to the loss of heterochromatin, compromising transcription and potentially contributing to decreased transcriptional synthesis as nuclei swell in aging.

This domain life cycle model challenges the traditional framework of chromatin regulation(5, 6, 8-11, 37, 38, 83). The simplistic view that dense chromatin (heterochromatin) suppresses gene expression while loose chromatin (euchromatin) facilitates it fails to capture the complexity observed at the nanoscale. Traditional models evolved from low-resolution techniques like wide-field microscopy and confocal microscopy, which could not identify the finer nanoscopic features of chromatin within the nucleus. Instead, they identified high-density (heterochromatin) and low-density (euchromatin) regions at the micron-scale(6)(8). These models influenced correlative interpretations of Hi-C connectivity (A- and B-compartments) and ChIP-Seq segmentation (hetero- and eu-chromatin)(6, 7). However, these approaches lacked the nanoscale information on conformationally defined chromatin packing domain life-cycle that only became available through the advent of ChromEM(25). The findings disclosed herein, rooted in the mass-fractal geometry of packing domains, demonstrate that chromatin regulation involves continuous transitions between dense cores, intermediate zones (active polymerases), and low-density outer zone, forming a unified, dynamic structure. If chromatin were governed solely by self-attractions (A to A, B to B), one would likely observe discrete, functionally independent condensates(9, 10). Instead, this model offers a more cohesive explanation of chromatin behavior, integrating transcriptional regulation with structural dynamics.

Finally, one can consider the implications of these findings to the concepts of transcriptional memory and the manipulation of cell behavior. Features of the packing domain life cycle echo reinforcement learning in computational networks(84). A transcriptional stimulus forms a nascent domain, and sustained signaling (positive reinforcement) with the right physical context matures the structure. This indicates that sustained, strong transcriptional signals will become physically encoded into the physical organization of a packing domain (FIG. 3). Once matured, this domain becomes a fixed physical object akin to a ‘transcriptional memory’, predisposing or preventing alternative configurations that would need the genomic segments allocated to the formed domain in future responses. Encoding transcriptional memory in 3D chromatin domains solves several problems: the analog nature of domains as a source of information and multiple degrees of freedom afforded by the processes that regulate the rate of domain formation for a given level of nascent domain-generating transcriptional activity (e.g., nuclear ionic environment, crowding, availability of histone modifying enzymes) may increase the flexibility of the system. It allows for long-term memory through domain stabilization while retaining the possibility of reprogramming through domain degradation; and the reinforcement learning properties of the system may facilitate, if necessary, cells' continuous responding to changing stressors and stimuli. However, the 3D nature of the information encoding presents a new problem: a 3D structure per se cannot be propagated through cell division. Epigenetic histone modifications may help solve this problem. Although epigenetic modifications as a mechanism to pass transcriptional information through cell division is well established, the prevailing context has centered around epigenetic modifications as the primary and original source of transcriptional information.

The results presented in this work may point out to a possibility of another complementary origin of epigenetic information that may originate from the primary process of chromatin domain formation (new memory=new domain), which subsequently guides the deposition of histone marks. In this context, epigenetic marks can be thought of as projecting the information encoded by 3D chromatin domains into the 1D epigenetically marked DNA sequence. In turn, since epigenetic marks can be heritable through mitosis, the marks may carry with them information about the genomic locations of chromatin domains into daughter cells, thus creating a long-term transcriptional memory, repeating the cycle and solving the problem of the heritance of 3D chromatin domains as elements of transcriptional memory. Paired with information on the location of a mature domain core, tools that allow the deposition of heterochromatin could potentially accelerate the activation of one or several genes.

This addresses a conundrum of epigenetic regulation of transcription is that most epigenetic regulators are gene sequence agnostic, and still, epigenetic modifications may result in reproducible and specific transcriptional outcomes. A part of the answer might be in conformationally defined chromatin domains. Through excluded volume effects, chromatin domains might be able to add gene specificity to otherwise nonspecific epigenetic regulators, as, for instance, they would be expected to act differently inside domain cores versus transcriptionally active ideal zones. In other words, these results suggest a hypothesis that chromatin conformation into domains indues geometric specificity instead of segment specificity to create a regulatory scheme independent of sequence. It might be instructive to distinguish the two types of epigenomic memory, one stored by histone and DNA modifications versus transcriptional memory, which arises in part but not solely due to epigenetic modifications. This work raises new questions of the deleterious consequences of a malformed transcriptional memory. It's possible that a sustained signal in the wrong context could produce physically-encoded programs resulting in pathological cellular states. Moreover, it highlights the potential for sequential stimuli (e.g., TNF followed by IL-12 exposure) to generate different transcriptional outcomes compared to concurrent stimuli (TNF+IL-12 together). By removing or inserting domain cores prior to these processes, it is worth considering if transcriptional memory could be manipulated in different disease contexts. These insights could inform the understanding of disease processes, such as chronic inflammation or cancer, where improper domain maturation locks cells into deleterious transcriptional states(85, 86).

### REFERENCES

1. Y. Shin, C. P. Brangwynne, Liquid phase condensation in cell physiology and disease. American Association for the Advancement of Science [Preprint] (2017). doi.org/10.1126/science.aaf4382.
2. D. Husmann, O. Gozani, Histone lysine methyltransferases in biology and disease. Nature Publishing Group [Preprint] (2019). doi.org/10.1038/s41594-019-0298-7.
3. J. I. Nakayama, T. Hayakawa, Physiological roles of class i HDAC complex and histone demethylase. [Preprint] (2011). doi.org/10.1155/2011/129383.
4. C. Arnould, V. Rocher, F. Saur, A. S. Bader, F. Muzzopappa, S. Collins, E. Lesage, B. Le Bozec, N. Puget, T. Clouaire, T. Mangeat, R. Mourad, N. Ahituv, D. Noordermeer, F. Erdel, M. Bushell, A. Marnef, G. Legube, Chromatin compartmentalization regulates the response to DNA damage. Nature 623, 183-192 (2023).
5. E. H. Finn, T. Misteli, Molecular basis and biological function of variability in spatial genome organization. Science (1979) 365 (2019).
6. E. Lieberman-Aiden, N. L. van Berkum, L. Williams, M. Imakaev, T. Ragoczy, A. Telling, I. Amit, B. R. Lajoie, P. J. Sabo, M. O. Dorschner, R. Sandstrom, B. Bernstein, M. A. Bender, M. Groudine, A. Gnirke, J. Stamatoyannopoulos, L. A. Mirny, E. S. Lander, J. Dekker, Comprehensive Mapping of Long-Range Interactions Reveals Folding Principles of the Human Genome. Science (1979) 326, 289-293 (2009).
7. S. S. P. Rao, M. H. Huntley, N. C. Durand, E. K. Stamenova, I. D. Bochkov, J. T. Robinson, A. L. Sanborn, I. Machol, A. D. Omer, E. S. Lander, E. L. Aiden, A 3D Map of the Human Genome at Kilobase Resolution Reveals Principles of Chromatin Looping. Cell 159, 1665-1680 (2014).
8 M. Falk, Y. Feodorova, N. Naumova, M. Imakaev, B. R. Lajoie, H. Leonhardt, B. Joffe, J. Dekker, G. Fudenberg, I. Solovei, L. A. Mirny, Heterochromatin drives compartmentalization of inverted and conventional nuclei. Nature 570, 395-399 (2019).
9. K. Polovnikov, S. Belan, M. Imakaev, H. B. Brandão, L. A. Mirny, A fractal polymer with loops recapitulates key features of chromosome organization. doi: 10.1101/2022.02.01.478588 (2022).
10. G. Fudenberg, M. Imakaev, C. Lu, A. Goloborodko, N. Abdennur, L. A. Mirny, Formation of Chromosomal Domains by Loop Extrusion. Cell Rep 15, 2038-2049 (2016).
11. J. Nuebler, G. Fudenberg, M. Imakaev, N. Abdennur, L. A. Mirny, Chromatin organization by an interplay of loop extrusion and compartmental segregation. Proceedings of the National Academy of Sciences 115 (2018).
12. S. Fujishiro, M. Sasai, Generation of dynamic three-dimensional genome structure through phase separation of chromatin. Proceedings of the National Academy of Sciences 119 (2022).
13. O. Adame-Arana, G. Bajpai, D. Lorber, T. Volk, S. Safran, Regulation of chromatin microphase separation by binding of protein complexes. Elife 12 (2023).
14. G. Shi, D. Thirumalai, From Hi-C Contact Map to Three-Dimensional Organization of Interphase Human Chromosomes. Phys Rev X 11, 011051 (2021).
15. Q. Szabo, F. Bantignies, G. Cavalli, Principles of genome folding into topologically associating domains. Sci Adv 5 (2019).
16. C. L. Woodcock, R. P. Ghosh, Chromatin Higher-order Structure and Dynamics. Cold Spring Harb Perspect Biol 2, a000596-a000596 (2010).
17. I. M. Baker, J. P. Smalley, K. A. Sabat, J. T. Hodgkinson, S. M. Cowley, Comprehensive Transcriptomic Analysis of Novel Class I HDAC Proteolysis Targeting Chimeras (PROTACs). Biochemistry 62, 645-656 (2023).
18. C. B. Greer, Y. Tanaka, Y. J. Kim, P. Xie, M. Q. Zhang, I.-H. Park, T. H. Kim, Histone Deacetylases Positively Regulate Transcription through the Elongation Machinery. Cell Rep 13, 1444-1455 (2015).
19. O. M. Dovey, C. T. Foster, N. Conte, S. A. Edwards, J. M. Edwards, R. Singh, G. Vassiliou, A. Bradley, S. M. Cowley, Histone deacetylase 1 and 2 are essential for normal T-cell development and genomic stability in mice. Blood 121, 1335-1344 (2013).
20. B. E. Bernstein, T. S. Mikkelsen, X. Xie, M. Kamal, D. J. Huebert, J. Cuff, B. Fry, A. Meissner, M. Wernig, K. Plath, R. Jaenisch, A. Wagschal, R. Feil, S. L. Schreiber, E. S. Lander, A Bivalent Chromatin Structure Marks Key Developmental Genes in Embryonic Stem Cells. Cell 125, 315-326 (2006).
21. N. Liu, M. Fromm, Z. Avramova, H3K27me3 and H3K4me3 Chromatin Environment at Super-Induced Dehydration Stress Memory Genes of Arabidopsis thaliana. Mol Plant 7, 502-513 (2014).
22. Z. Wang, A. G. Chivu, L. A. Choate, E. J. Rice, D. C. Miller, T. Chu, S.-P. Chou, N. B. Kingsley, J. L. Petersen, C. J. Finno, R. R. Bellone, D. F. Antczak, J. T. Lis, C. G. Danko, Prediction of histone post-translational modification patterns based on nascent transcription data. Nat Genet 54, 295-305 (2022).
23. S. S. P. Rao, S.-C. Huang, B. Glenn St Hilaire, J. M. Engreitz, E. M. Perez, K.-R. Kieffer-Kwon, A. L. Sanborn, S. E. Johnstone, G. D. Bascom, I. D. Bochkov, X. Huang, M. S. Shamim, J. Shin, D. Turner, Z. Ye, A. D. Omer, J. T. Robinson, T. Schlick, B. E. Bernstein, R. Casellas, E. S. Lander, E. L. Aiden, Cohesin Loss Eliminates All Loop Domains. Cell 171, 305-320.e24 (2017).
24. E. Miron, R. Oldenkamp, J. M. Brown, D. M. S. Pinto, C. S. Xu, A. R. Faria, H. A. Shaban, J. D. P. Rhodes, C. Innocent, S. de Ornellas, H. F. Hess, V. Buckle, L. Schermelleh, Chromatin arranges in chains of mesoscale domains with nanoscale functional topography independent of cohesin. Sci Adv 6 (2020).
25. H. D. Ou, S. Phan, T. J. Deerinck, A. Thor, M. H. Ellisman, C. C. O'Shea, ChromEMT: Visualizing 3D chromatin structure and compaction in interphase and mitotic cells. Science (1979) 357 (2017).
26. Q. Szabo, A. Donjon, I. Jerković, G. L. Papadopoulos, T. Cheutin, B. Bonev, E. P. Nora, B. G. Bruneau, F. Bantignies, G. Cavalli, Regulation of single-cell genome organization into TADs and chromatin nanodomains. Nat Genet 52, 1151-1157 (2020).
27. J. M. Luppino, D. S. Park, S. C. Nguyen, Y. Lan, Z. Xu, R. Yunker, E. F. Joyce, Cohesin promotes stochastic domain intermingling to ensure proper regulation of boundary-proximal genes. Nat Genet 52, 840-848 (2020).
28. M. V. Neguembor, L. Martin, A. Castells-García, P. A. Gómez-García, C. Vicario, D. Carnevali, J. AlHaj Abed, A. Granados, R. Sebastian-Perez, F. Sottile, J. Solon, C. ting Wu, M. Lakadamyali, M. P. Cosma, Transcription-mediated supercoiling regulates genome folding and loop formation. Mol Cell 81, 3065-3081.e12 (2021).
29. Y. Li, A. Eshein, R. K. A. Virk, A. Eid, W. Wu, J. Frederick, D. VanDerway, S. Gladstein, K. Huang, A. R. Shim, N. M. Anthony, G. M. Bauer, X. Zhou, V. Agrawal, E. M. Pujadas, S. Jain, G. Esteve, J. E. Chandler, T.-Q. Nguyen, R. Bleher, J. J. de Pablo, I. Szleifer, V. P. Dravid, L. M. Almassalha, V. Backman, Nanoscale chromatin imaging and analysis platform bridges 4D chromatin organization with molecular function. Sci Adv 7 (2021).
30. B. Bintu, L. J. Mateo, J.-H. Su, N. A. Sinnott-Armstrong, M. Parker, S. Kinrot, K. Yamaya, A. N. Boettiger, X. Zhuang, Super-resolution chromatin tracing reveals domains and cooperative interactions in single cells. Science (1979) 362 (2018).
31. A. N. Boettiger, B. Bintu, J. R. Moffitt, S. Wang, B. J. Beliveau, G. Fudenberg, M. Imakaev, L. A. Mirny, C. Wu, X. Zhuang, Super-resolution imaging reveals distinct chromatin folding for different epigenetic states. Nature 529, 418-422 (2016).
32. A. Hafner, M. Park, S. E. Berger, S. E. Murphy, E. P. Nora, A. N. Boettiger, Loop stacking organizes genome folding from TADs to chromosomes. Mol Cell 83, 1377-1392.e6 (2023).
33. J. Otterstrom, A. Castells-Garcia, C. Vicario, P. A. Gomez-Garcia, M. P. Cosma, M. Lakadamyali, Super-resolution microscopy reveals how histone tail acetylation affects DNA compaction within nucleosomes in vivo. Nucleic Acids Res 47, 8470-8484 (2019).
34. M. A. Ricci, C. Manzo, M. F. García-Parajo, M. Lakadamyali, M. P. Cosma, Chromatin Fibers Are Formed by Heterogeneous Groups of Nucleosomes In Vivo. Cell 160, 1145-1158 (2015).
35. T. Nozaki, S. Shinkai, S. Ide, K. Higashi, S. Tamura, M. A. Shimazoe, M. Nakagawa, Y. Suzuki, Y. Okada, M. Sasai, S. Onami, K. Kurokawa, S. Iida, K. Maeshima, Condensed but liquid-like domain organization of active chromatin regions in living human cells. Sci Adv 9 (2023).
36. Y. Li, V. Agrawal, R. K. A. Virk, E. Roth, W. S. Li, A. Eshein, J. Frederick, K. Huang, L. Almassalha, R. Bleher, M. A. Carignano, I. Szleifer, V. P. Dravid, V. Backman, Analysis of three-dimensional chromatin packing domains by chromatin scanning transmission electron microscopy (ChromSTEM). Sci Rep 12, 12198 (2022).
37. L. A. Mirny, The fractal globule as a model of chromatin architecture in the cell. Chromosome Research 19, 37-51 (2011).
38. G. Fudenberg, G. Getz, M. Meyerson, L. A. Mirny, High order chromatin architecture shapes the landscape of chromosomal alterations in cancer. Nat Biotechnol 29, 1109-1113 (2011).
39. K. E. Polovnikov, M. Gherardi, M. Cosentino-Lagomarsino, M. V. Tamm, Fractal Folding and Medium Viscoelasticity Contribute Jointly to Chromosome Dynamics. Phys Rev Lett 120, 088101 (2018).
40. M. V. Tamm, L. I. Nazarov, A. A. Gavrilov, A. V. Chertovich, Anomalous Diffusion in Fractal Globules. Phys Rev Lett 114, 178102 (2015).
41. G. Forte, A. Buckle, S. Boyle, D. Marenduzzo, N. Gilbert, C. A. Brackley, Transcription modulates chromatin dynamics and locus configuration sampling. Nat Struct Mol Biol 30, 1275-1285 (2023).
42. K. Huang, Y. Li, A. R. Shim, R. K. A. Virk, V. Agrawal, A. Eshein, R. J. Nap, L. M. Almassalha, V. Backman, I. Szleifer, Physical and data structure of 3D genome. Sci Adv 6 (2020).
43. T.-H. S. Hsieh, C. Cattoglio, E. Slobodyanyuk, A. S. Hansen, X. Darzacq, R. Tjian, Enhancer-promoter interactions and transcription are largely maintained upon acute loss of CTCF, cohesin, WAPL or YY1. Nat Genet 54, 1919-1932 (2022).
44. M. Gabriele, H. B. Brandão, S. Grosse-Holz, A. Jha, G. M. Dailey, C. Cattoglio, T.-H. S. Hsieh, L. Mirny, C. Zechner, A. S. Hansen, Dynamics of CTCF- and cohesin-mediated chromatin looping revealed by live-cell imaging. Science (1979) 376, 496-501 (2022).
45. Y. Liu, J. Dekker, CTCF-CTCF loops and intra-TAD interactions show differential dependence on cohesin ring integrity. Nat Cell Biol 24, 1516-1527 (2022).
46. M. A. Carignano, M. Kroeger, L. M. Almassalha, V. Agrawal, W. S. Li, E. M. Pujadas-Liwag, R. J. Nap, V. Backman, I. Szleifer, Local volume concentration, packing domains, and scaling properties of chromatin. Elife 13 (2024).
47. H. Matsuda, G. G. Putzel, V. Backman, I. Szleifer, Macromolecular Crowding as a Regulator of Gene Transcription. Biophys J 106, 1801-1810 (2014).
48. G. G. Putzel, M. Tagliazucchi, I. Szleifer, Nonmonotonic Diffusion of Particles Among Larger Attractive Crowding Spheres. Phys Rev Lett 113, 138302 (2014).
49. K. Maeshima, K. Kaizu, S. Tamura, T. Nozaki, T. Kokubo, K. Takahashi, The physical size of transcription factors is key to transcriptional regulation in chromatin domains. Journal of Physics: Condensed Matter 27, 064116 (2015).
50. J. Jumper, R. Evans, A. Pritzel, T. Green, M. Figurnov, O. Ronneberger, K. Tunyasuvunakool, R. Bates, A. Žídek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature 596, 583-589 (2021).
51. R. K. A. Virk, W. Wu, L. M. Almassalha, G. M. Bauer, Y. Li, D. VanDerway, J. Frederick, D. Zhang, A. Eshein, H. K. Roy, I. Szleifer, V. Backman, Disordered chromatin packing regulates phenotypic plasticity. Sci Adv 6 (2020).
52. L. M. Almassalha, A. Tiwari, P. T. Ruhoff, Y. Stypula-Cyrus, L. Cherkezyan, H. Matsuda, M. A. Dela Cruz, J. E. Chandler, C. White, C. Maneval, H. Subramanian, I. Szleifer, H. K. Roy, V. Backman, The Global Relationship between Chromatin Physical Topology, Fractal Structure, and Gene Expression. Sci Rep 7, 41061 (2017).
53. L. M. Almassalha, G. M. Bauer, W. Wu, L. Cherkezyan, D. Zhang, A. Kendra, S. Gladstein, J. E. Chandler, D. VanDerway, B.-L. L. Seagle, A. Ugolkov, D. D. Billadeau, T. V. O'Halloran, A. P. Mazar, H. K. Roy, I. Szleifer, S. Shahabi, V. Backman, Macrogenomic engineering via modulation of the scaling of chromatin packing density. Nat Biomed Eng 1, 902-913 (2017).
54. C. Tan, S. Saurabh, M. P. Bruchez, R. Schwartz, P. LeDuc, Molecular crowding shapes gene expression in synthetic cellular nanosystems. Nat Nanotechnol 8, 602-608 (2013).
55. Y. Lorch, R. D. Kornberg, B. Maier-Davis, Role of the histone tails in histone octamer transfer. Nucleic Acids Res 51, 3671-3678 (2023).
56. K. Maeshima, T. Matsuda, Y. Shindo, H. Imamura, S. Tamura, R. Imai, S. Kawakami, R. Nagashima, T. Soga, H. Noji, K. Oka, T. Nagai, A Transient Rise in Free Mg2+ Ions Released from ATP-Mg Hydrolysis Contributes to Mitotic Chromosome Condensation. Current Biology 28, 444-451.e6 (2018).
57. A. D. Stephens, P. Z. Liu, V. Kandula, H. Chen, L. M. Almassalha, C. Herman, V. Backman, T. O'Halloran, S. A. Adam, R. D. Goldman, E. J. Banigan, J. F. Marko, Physicochemical mechanotransduction alters nuclear shape and mechanics via heterochromatin formation. Mol Biol Cell 30, 2320-2330 (2019).
58. K. Nishimura, T. Fukagawa, H. Takisawa, T. Kakimoto, M. Kanemaki, An auxin-based degron system for the rapid depletion of proteins in nonplant cells. Nat Methods 6, 917-922 (2009).
59. A. Yesbolatova, Y. Saito, N. Kitamoto, H. Makino-Itou, R. Ajima, R. Nakano, H. Nakaoka, K. Fukui, K. Gamo, Y. Tominari, H. Takeuchi, Y. Saga, K. Hayashi, M. T. Kanemaki, The auxin-inducible degron 2 technology provides sharp degradation control in yeast, mammalian cells, and mice. Nat Commun 11, 5701 (2020).
60. O. Bensaude, Inhibiting eukaryotic transcription. Which compound to choose? How to evaluate its activity? Transcription 2, 103-108 (2011).
61. S. Gladstein, L. M. Almassalha, L. Cherkezyan, J. E. Chandler, A. Eshein, A. Eid, D. Zhang, W. Wu, G. M. Bauer, A. D. Stephens, S. Morochnik, H. Subramanian, J. F. Marko, G. A. Ameer, I. Szleifer, V. Backman, Multimodal interference-based imaging of nanoscale structure and macromolecular motion uncovers UV induced cellular paroxysm. Nat Commun 10, 1652 (2019).
62. L. M. Almassalha, G. M. Bauer, J. E. Chandler, S. Gladstein, L. Cherkezyan, Y. Stypula-Cyrus, S. Weinberg, D. Zhang, P. Thusgaard Ruhoff, H. K. Roy, H. Subramanian, N. S. Chandel, I. Szleifer, V. Backman, Label-free imaging of the native, living cellular nanoarchitecture using partial-wave spectroscopic microscopy. Proceedings of the National Academy of Sciences 113 (2016).
63. A. Castells-Garcia, I. Ed-daoui, E. González-Almela, C. Vicario, J. Ottestrom, M. Lakadamyali, M. V. Neguembor, M. P. Cosma, Super resolution microscopy reveals how elongating RNA polymerase II and nascent RNA interact with nucleosome clutches. Nucleic Acids Res 50, 175-190 (2022).
64. S. J. Heo, S. Thakur, X. Chen, C. Loebel, B. Xia, R. McBeath, J. A. Burdick, V. B. Shenoy, R. L. Mauck, M. Lakadamyali, Aberrant chromatin reorganization in cells from diseased fibrous connective tissue in response to altered chemomechanical cues. Nat Biomed Eng 7, 177-191 (2023).
65. E. A. Bowman, W. G. Kelly, RNA Polymerase II transcription elongation and Pol II CTD Ser2 phosphorylation. Nucleus 5, 224-236 (2014).
66. T. Yu, Y. Wang, Q. Hu, W. Wu, Y. Wu, W. Wei, D. Han, Y. You, N. Lin, N. Liu, “The EZH2 inhibitor GSK343 suppresses cancer stem-like phenotypes and reverses mesenchymal transition in glioma cells;” www.impactjournals.com/oncotarget.
67. B. C. Hitz, J.-W. Lee, O. Jolanki, M. S. Kagda, K. Graham, P. Sud, I. Gabdank, J. S. Strattan, C. A. Sloan, T. Dreszer, L. D. Rowe, N. R. Podduturi, V. S. Malladi, E. T. Chan, J. M. Davidson, M. Ho, S. Miyasato, M. Simison, F. Tanaka, Y. Luo, I. Whaling, E. L. Hong, B. T. Lee, R. Sandstrom, E. Rynes, J. Nelson, A. Nishida, A. Ingersoll, M. Buckley, M. Frerker, D. S. Kim, N. Boley, D. Trout, A. Dobin, S. Rahmanian, D. Wyman, G. Balderrama-Gutierrez, F. Reese, N. C. Durand, O. Dudchenko, D. Weisz, S. S. P. Rao, A. Blackburn, D. Gkountaroulis, M. Sadr, M. Olshansky, Y. Eliaz, D. Nguyen, I. Bochkov, M. S. Shamim, R. Mahajan, E. Aiden, T. Gingeras, S. Heath, M. Hirst, W. J. Kent, A. Kundaje, A. Mortazavi, B. Wold, J. M. Cherry, The ENCODE Uniform Analysis Pipelines “Elements of Order 4404 E Oregon St, Bellingham WA 98226, USA. doi: 10.1101/2023.04.04.535623.
68. An integrated encyclopedia of DNA elements in the human genome. Nature 489, 57-74 (2012).
69. Y. Luo, B. C. Hitz, I. Gabdank, J. A. Hilton, M. S. Kagda, B. Lam, Z. Myers, P. Sud, J. Jou, K. Lin, U. K. Baymuradov, K. Graham, C. Litton, S. R. Miyasato, J. S. Strattan, O. Jolanki, J.-W. Lee, F. Y. Tanaka, P. Adenekan, E. O'Neill, J. M. Cherry, New developments on the Encyclopedia of DNA Elements (ENCODE) data portal. Nucleic Acids Res 48, D882-D889 (2020).
70. R. U. Pathak, M. Soujanya, R. K. Mishra, Deterioration of nuclear morphology and architecture: A hallmark of senescence and aging. Ageing Res Rev 67, 101264 (2021).
71. A. Brandt, G. Krohne, J. Großhans, The farnesylated nuclear proteins KUGELKERN and LAMIN B promote aging-like phenotypes in Drosophila flies. Aging Cell 7, 541-551 (2008).
72. J. Kang, D. I. Benjamin, S. Kim, J. S. Salvi, G. Dhaliwal, R. Lam, A. Goshayeshi, J. O. Brett, L. Liu, T. A. Rando, Depletion of SAM leading to loss of heterochromatin drives muscle stem cell ageing. Nat Metab 6, 153-168 (2024).
73. J.-H. Lee, E. W. Kim, D. L. Croteau, V. A. Bohr, Heterochromatin: an epigenetic point of view in aging. Exp Mol Med 52, 1466-1474 (2020).
74. D. Nicetto, K. S. Zaret, Role of H3K9me3 heterochromatin in cell identity establishment and maintenance. Curr Opin Genet Dev 55, 1-10 (2019).
75. E. Benz, A. Pinel, C. Guillet, F. Capel, B. Pereira, M. De Antonio, M. Pouget, A. J. Cruz-Jentoft, D. Eglseer, E. Topinkova, R. Barazzoni, F. Rivadeneira, M. A. Ikram, M. Steur, T. Voortman, J. D. Schoufour, P. J. M. Weijs, Y. Boirie, Sarcopenia and Sarcopenic Obesity and Mortality Among Older People. JAMA Netw Open 7, e243604 (2024).
76. C. Beaudart, C. Demonceau, J. Reginster, M. Locquet, M. Cesari, A. J. Cruz Jentoft, O. Bruyère, Sarcopenia and health-related quality of life: A systematic review and meta-analysis. J Cachexia Sarcopenia Muscle 14, 1228-1243 (2023).
77. J. Xu, C. S. Wan, K. Ktoris, E. M. Reijnierse, A. B. Maier, Sarcopenia Is Associated with Mortality in Adults: A Systematic Review and Meta-Analysis. Gerontology 68, 361-376 (2022).
78. Z. Yang, K. L. MacQuarrie, E. Analau, A. E. Tyler, F. J. Dilworth, Y. Cao, S. J. Diede, S. J. Tapscott, MyoD and E-protein heterodimers switch rhabdomyosarcoma cells from an arrested myoblast phase to a differentiated state. Genes Dev 23, 694-707 (2009).
79. Y. Cao, Z. Yao, D. Sarkar, M. Lawrence, G. J. Sanchez, M. H. Parker, K. L. MacQuarrie, J. Davison, M. T. Morgan, W. L. Ruzzo, R. C. Gentleman, S. J. Tapscott, Genome-wide MyoD Binding in Skeletal Muscle Cells: A Potential for Broad Cellular Reprogramming. Dev Cell 18, 662-674 (2010).
80. Y. Nabeshima, K. Hanaoka, M. Hayasaka, E. Esuml, S. Li, I. Nonaka, Y. Nabeshima, Myogenin gene disruption results in perinatal lethality because of severe muscle defect. Nature 364, 532-535 (1993).
81. J. M. Hernández-Hernández, E. G. García-González, C. E. Brun, M. A. Rudnicki, The myogenic regulatory factors, determinants of muscle development, cell identity and regeneration. Semin Cell Dev Biol 72, 10-18 (2017).
82. V. R. Kedlian, Y. Wang, T. Liu, X. Chen, L. Bolt, C. Tudor, Z. Shen, E. S. Fasouli, E. Prigmore, V. Kleshchevnikov, J. P. Pett, T. Li, J. E. G. Lawrence, S. Perera, M. Prete, N. Huang, Q. Guo, X. Zeng, L. Yang, K. Polański, N.-J. Chipampe, M. Dabrowska, X. Li, O. A. Bayraktar, M. Patel, N. Kumasaka, K. T. Mahbubani, A. P. Xiang, K. B. Meyer, K. Saeb-Parsy, S. A. Teichmann, H. Zhang, Human skeletal muscle aging atlas. Nat Aging, doi: 10.1038/s43587-024-00613-3 (2024).
83. W. Schwarzer, N. Abdennur, A. Goloborodko, A. Pekowska, G. Fudenberg, Y. Loe-Mie, N. A. Fonseca, W. Huber, C. H. Haering, L. Mirny, F. Spitz, Two independent modes of chromatin organization revealed by cohesin removal. Nature 551, 51-56 (2017).
84. L. P. Kaelbling, M. L. Littman, A. W. Moore, Reinforcement Learning: A Survey. Journal of Artificial Intelligence Research 4, 237-285 (1996).
85. I. Mitroulis, K. Ruppova, B. Wang, L.-S. Chen, M. Grzybek, T. Grinenko, A. Eugster, M. Troullinaki, A. Palladini, I. Kourtzelis, A. Chatzigeorgiou, A. Schlitzer, M. Beyer, L. A. B. Joosten, B. Isermann, M. Lesche, A. Petzold, K. Simons, I. Henry, A. Dahl, J. L. Schultze, B. Wielockx, N. Zamboni, P. Mirtschink, Ü. Coskun, G. Hajishengallis, M. G. Netea, T. Chavakis, Modulation of Myelopoiesis Progenitors Is an Integral Component of Trained Immunity. Cell 172, 147-161.e12 (2018).
86. V. Parreno, V. Loubiere, B. Schuettengruber, L. Fritsch, C. C. Rawal, M. Erokhin, B. Gyõrffy, D. Normanno, M. Di Stefano, J. Moreaux, N. L. Butova, I. Chiolo, D. Chetverina, A.-M. Martinez, G. Cavalli, Transient loss of Polycomb components induces an epigenetic cancer fate. Nature 629, 688-696 (2024).
87. E. R. H. Walter, M. A. Fox, D. Parker, J. A. G. Williams, Enhanced selectivity for Mg²⁺ with a phosphinate-based chelate: APDAP versus APTRA. Dalton Transactions 47, 1879-1887 (2018).
88. N. C. Durand, M. S. Shamim, I. Machol, S. S. P. Rao, M. H. Huntley, E. S. Lander, E. L. Aiden, Juicer Provides a One-Click System for Analyzing Loop-Resolution Hi-C Experiments. Cell Syst 3, 95-98 (2016).
89. R. H. van der Weide, T. van den Brand, J. H. I. Haarhuis, H. Teunissen, B. D. Rowland, E. de Wit, Hi-C analyses with GENOVA: a case study with cohesin variants. NAR Genom Bioinform 3 (2021).
90. N. Abdennur, L. A. Mirny, Cooler: scalable storage for Hi-C data and other genomically labeled arrays. Bioinformatics 36, 311-316 (2020).
91. N. Abdennur, S. Abraham, G. Fudenberg, I. M. Flyamer, A. A. Galitsyna, A. Goloborodko, M. Imakaev, B. A. Oksuz, S. V. Venev, Y. Xiao, Cooltools: Enabling high-resolution Hi-C analysis in Python. PLOS Comput Biol 20, e1012067 (2024).
92. M. Ovesný, P. Křížek, J. Borkovec, Z. Švindrych, G. M. Hagen, ThunderSTORM: a comprehensive ImageJ plug-in for PALM and STORM data analysis and super-resolution imaging. Bioinformatics 30, 2389-2390 (2014).
93. W. M. Brown, M. K. Petersen, S. J. Plimpton, G. S. Grest, Liquid crystal nanodroplets in solution. J Chem Phys 130 (2009).
94. A. P. Thompson, H. M. Aktulga, R. Berger, D. S. Bolintineanu, W. M. Brown, P. S. Crozier, P. J. in 't Veld, A. Kohlmeyer, S. G. Moore, T. D. Nguyen, R. Shan, M. J. Stevens, J. Tranchida, C. Trott, S. J. Plimpton, LAMMPS—a flexible simulation tool for particle-based materials modeling at the atomic, meso, and continuum scales. Comput Phys Commun 271, 108171 (2022).

### Example 2

Multicellular organisms are shaped not only by their genes but by the ability to execute cell specific transcriptional programs to reach new states, from differentiation into myriad cell types to coherent responses to novel stimuli. Above is described how genome geometry is physically encoded into several thousand nanoscopic chromatin packing domains, each serving as the physical element of cellular transcriptional memory. Each domain encompasses heterochromatin and euchromatin into a single, cohesive power-law geometric unit guided by, and enhancing, transcription. This organization raises a regulatory paradox: the dense structure of heterochromatin facilitates coherent transcriptional activation patterns to emerge. In this example, this paradox is demonstrated to be solved by the geometric positioning of non-transcribed ‘junk’ (introns and intergenic) DNA with exons to produce surface-area to volume assemblies observed as packing domains. This redefines ‘junk’ into ‘volumetric’ DNA, showing a crucial function to support the transcription of complex genes, provide physical durability, and act as a non-mutational system for genomic sampling by optimizing the complexity of genomic space. This geometric encoding is then demonstrated to parallel the emergence of body-plan complexity, transitioning from linear geometry in S. cerevisiae to power-law geometries in complex metazoans. These findings generate new avenues of research in chromatin structure-function spanning aging, evolution, and development.

### Materials and Methods

### Geometric Positioning of Exons and Volumetric DNA in Relation to Packing Domains

Two distinct derivations are considered to assess if junk is coupled with exons to generate surface area to volume assemblies. The first derivation is as follows and concludes that D is inversely related to γ. In either derivation, the property of junk remains volumetric and exons behave as a wavy line on a reaction surface.

### Derivation 1)

Chromatin packing domains are nanoscopic, heterogeneous mass-fractal structures. The transformation of a chromatin chain into volume is defined by how mass, M, scales as a function of the radial distance, r by M˜r^{D} where D is the fractal dimension. The chromatin volume fraction, φ, at the radial distance, r, is defined by:

$$ϕ ( r ) = {{ϕ}_{0} ( \frac{{r}_{c}}{r} )}^{3 - D} , \qquad 5.$$

where φ₀ is the chromatin volume fraction at r=0 (domain center) and r_{c} is the chain radius^{2, 3, 52} The total amount of chromatin in basepairs within the domain volume will therefore be:

$$N ( r ) = {N}_{c} * {{A}_{D} ( \frac{r}{{r}_{c}} )}^{D} , \qquad 6.$$

with N being the basepairs contained within the radius, N_{c} the number of basepairs within the chain and A_{D} the packing efficiency of the chain within a domain. Noting that A_{D}=1 indicates efficient packing throughout the domain volume.

For hard 3-D objects (e.g. a hard sphere), the number of basepairs on the surface within a shell, Nₛ, of radius ΔR that is much smaller than the radius of the volume (ΔR<<r) is:

$${N}_{s} ( r ) = \frac{dN}{dr} Δ R . \qquad 7.$$

Substituting in Eq. (6), we therefore observe:

$${N}_{s} ( r ) = D * {N}_{c} * {{A}_{D} ( \frac{r}{{r}_{c}} )}^{D - 1} \frac{Δ R}{{r}_{c}} . \qquad 8.$$

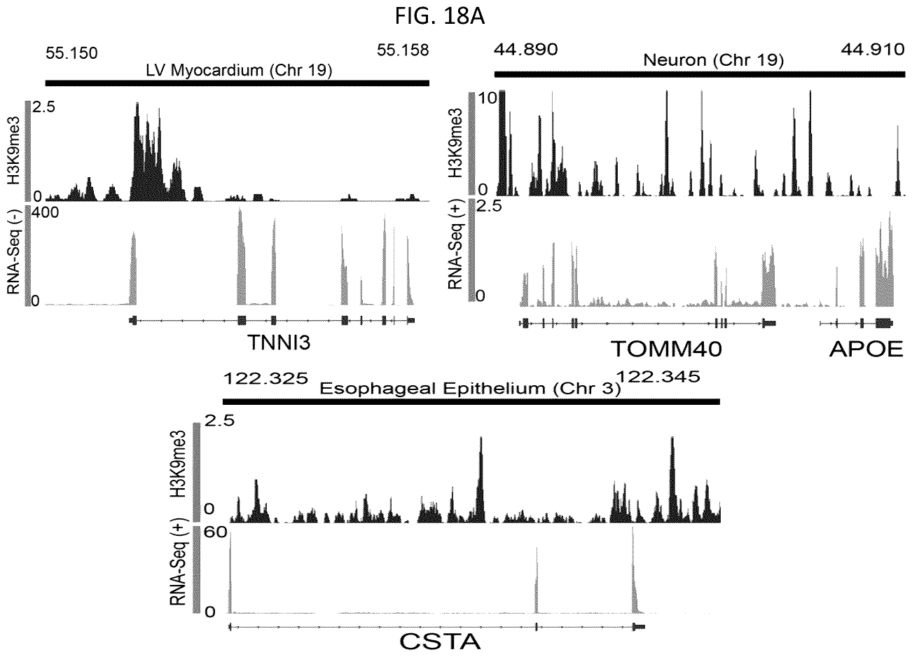

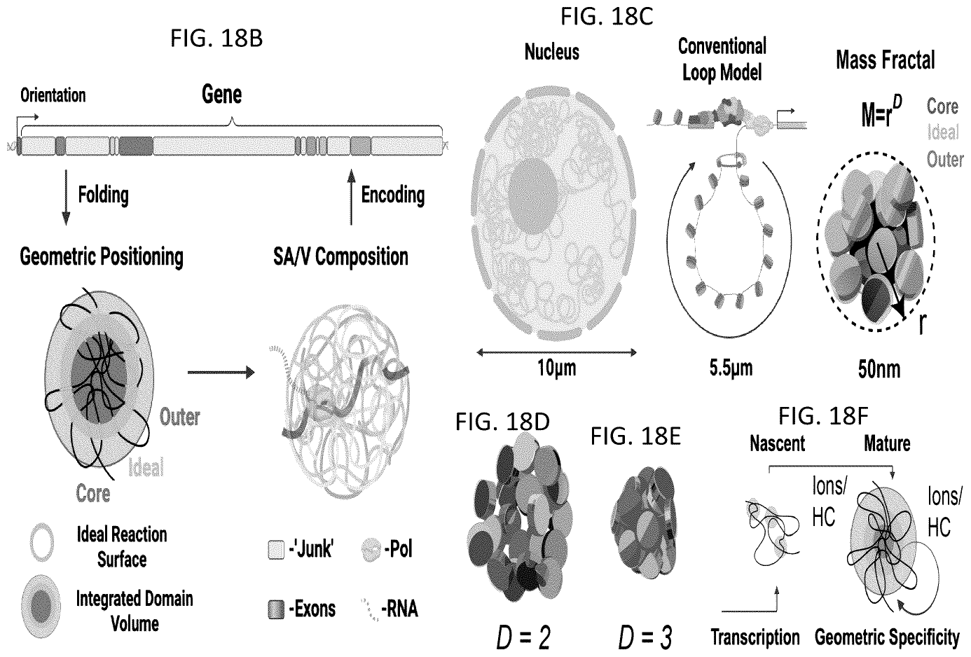

Accounting for the fact that the chain elements are a polymer that can go in and out of the hard shell (a wiggly line, FIG. 18), it is instead necessary to take the fractional derivative to capture the behavior at the surface:

$${N}_{s} ( r ) = \frac{dN}{{dr}^{β}} Δ {R}^{β} , \qquad 9.$$

where β is the order (dimension) of the derivative and ranges from 0<β<1.

Utilizing the chain rule for a function f(x):

$$\frac{df}{{dx}^{α}} = \frac{df}{dx} * \frac{dx}{{dx}^{α}} = \frac{1}{α} * {x}^{1 - α} \frac{df}{dx} . \qquad 10.$$

By Eq. (9) and Eq. (10), we therefore observe that the composition of the surface is:

$${N}_{s} ( r ) = \frac{1}{β} * {r}^{1 - β} \frac{dN}{dr} Δ {R}^{β} = \frac{D}{β} * {N}_{c} * {{A}_{D} ( \frac{r}{{r}_{c}} )}^{D - β} * {( \frac{Δ R}{{r}_{c}} )}^{β} . \qquad 11.$$

Assuming that the content of exons, E, in basepairs is approximately that of the contents of a domain ideal surface E∝N_{S} then the proportionality constant in this relationship is ∝β. That is to say that the surface is not exclusively exons but a wiggly line primarily made of exons. From this, we observe:

$$E ( r ) = D * k * {N}_{c} * {{A}_{D} ( \frac{r}{{r}_{c}} )}^{D - β} * {( \frac{Δ R}{{r}_{c}} )}^{β} , \qquad 12.$$

with k representing the fraction of the surface basepairs that are exons. Likewise, the length of a segment within a domain volume is the number of basepairs within that volume, L(r)=N(r). Transcriptional reactions occur at the ideal zone (goldilocks zone), the region where the balance between density stabilizes the intermediate complexes without overly limiting diffusivity of the reactant species^{6, 7, 38, 93}. The total length of the gene, L(r=R_{g1}), and the exons, E(r=R_{g1}) within domains to the goldilocks radius are:

$$L = {N}_{c} {{A}_{D} ( \frac{{R}_{gl}}{{r}_{c}} )}^{D} , \qquad 13.$$

which indicates that

$$( \frac{{R}_{gl}}{{r}_{c}} ) = {( L / {N}_{c} {A}_{D} )}^{1 / D} . \qquad 14.$$

Solving for E using Eq. (12) utilizing the relation from Eq. (14) the exon contents can be calculated by

$$15. E = D * k * ( {N}_{c} * {A}_{D} ) ( β / D ) * {δ}_{g l} * {( L )}^{( \frac{D - β}{D} )} with {δ}_{g l} = {( \frac{Δ {R}_{g l}}{{r}_{c}} )}^{β} .$$

For simplicity, we can now let

$$Y = D * k * ( {N}_{c} * {A}_{D} ) ( β / D ) * {δ}_{g l} and C = \frac{D - β}{D}$$

which simplifies Eq. (14) to

E=Y*L^{C} with C generally bounded between ⅔ and 1 due to the limits of D in cells of 2 to 3. 16.

The existence of power-law scaling between exon length, E, and total gene length, L suggests a scaling relationship between the intron/intergenic segment and the exon. An exponent, γ in Eq. (2) and Eq. (4) such that

$$I = \frac{{E}^{γ}}{m} ,$$

is observed.

$$If γ = 1 + \frac{1}{C} then I = \frac{{E}^{1 + 1 / C}}{m} ,$$

this would result in

$$\frac{I}{E} = \frac{{E}^{1 / C}}{m}$$

which by Eq. (15) becomes

$$\frac{I}{E} = \frac{{Y}^{1 / C} * L}{m} .$$

Given that Y is nearly 1, this results in

$$\frac{I}{E} ≅ \frac{{L}^{n}}{m}$$

consistent with the experimental observations in Eq. (1). Translating the power-law scaling described by γ into the mass-fractal dimension of domains, it is observed that

$$17. γ = 1 + \frac{1}{C} = 1 + \frac{D}{D - β} as described in Eq . ( 4 ) .$$

Positioning of elements are now considered depending on the property of the exon segments in relation to the surface elements defined by β. For β=1, the entire contents of the exon are within the hard shell. In contrast, for β=0, the exon portion is volumetrically distributed indicating that there is no geometry.

In the most likely case, where exons constitute a portion of the surface (a wiggly line) then 0<β<1 with D of domains ranging experimentally on ChromSTEM and in polymer-modeling between 2 and 3 (2<D<3)^{2, 3, 5}, this results in 2<γ<3 observed experimentally. When γ=2 and β=0, resulting in exons being distributed for any observed D (no geometric relationship). Conversely, when γ=3 and β=1, D=2 resulting in the limiting case of a chromatin polymer in a good solvent. The most frequently observed γ˜2.3 with a β˜0.7 (70% of the exons constitute the surface) then D˜2.8. It is worth noting that in this derivation the observed γ and m values plotted are those of the ensemble, and not the realized values for each individual gene. In essence, the entire genome is organized by this behavior, however, each individual gene may use segments from a neighboring segment in order to achieve the realized domain.

### Derivation 2)

It is well described that the chromatin domains as a mass-fractal behave as M˜r^{D} which is equivalent to L˜r^{D} as the generated mass within the domain is the chain of length, L, within the volume^{2, 3, 6, 37, 52}. By Eq. (1), we arrive at the following relationship:

$$18. \frac{E x o n}{Intron} ≅ \frac{m}{{r}^{D}} which in turn becomes {r}^{D} = Intron * \frac{m}{E x o n} .$$

In essence, this indicates that the intron length is proportional to the occupied fractal volume (r^{D}). Since m is less than or equal to exon length (and frequently the ratio ˜1), the generated domain volume is generated by the introns. Taking the relationship observed in Eq. (18) with Eq. (2), we arrive at:

$$19. r = {( \frac{m}{E x o n} * \frac{E x o {n}^{γ}}{m} )}^{( \frac{1}{D} )} which simplifies to {r}^{D} = E x o {n}^{( γ - 1 )} .$$

Taking the natural log and solving for γ as a function of D, we arrive at

$$20. γ = D * L o {g}_{E x o n} ( r ) + 1 .$$

Taking into account that the exons are a chain of N nucleosomes, this results in a linear segment of length N*r_{c}. In the limit where this is a chain forming the surface of a hard sphere (D=3), then the Exon length would approach β*4πr² where B is the fraction of the surface that is composed of exon and is bounded between 0 and 1. In the case where β is 0, there is no geometric relationship. Conversely, β=1 is consistent with the entire surface composed of exons. For the realized size of mature packing domains typically spanning between a radius of 50 to 150 nm, so long as the chain results in a β occupying at least 0.1% of the surface, then γ is bounded between 2 and 3 and scales with D. For a fractal surface, similar considerations can be derived as described above, however, the result is consistent with exons behaving as a wavy chain along a domain surface. It is worth noting that from Eq. (19) alone that exons scale as surface elements.

In either approach, junk DNA scales as a volumetric element whereas exon scales as generally as wavy chains on a domain surface. Thus, Derivation (1) is more likely owing to the scaling properties of individual genes, however, further experimental testing would be necessary to differentiate which transformation occurs.

### Power-Law Genomic Analysis

RefSeq genomes were obtained from Igv.org/app mapping to the UCSC genome browser assemblies. The selected genome assemblies were as follows: Human (GrCH38), Mouse (GRCm39), Zebrafish (GRCZ11), Drosophila melanogaster (dm6), and S. cerevisiae (sacCer3). These text files were converted to xlsx extensions and then imported into Mathematica v12 for subsequent analysis with custom-built code. Genes were then separated to analyze protein coding with assigned prefix “NM” and non-protein coding “NR”. The first isoform was selected for analysis of individual genes and compared to analysis with all isoforms. At the level of chromosome analysis, all isoforms were considered. Exon start and stop positions were used for segmentation and an reciprocal start/stop position for introns was generated. For simplicity, all exons were assumed to be part of the gene within the isoform variant. For chromosome wide analysis to account for the direction of transcriptional reading frames, genes were separated by their location to the positive or negative strand orientation for analysis. Multi-start or multi-stop exon overlapping events accounted for less than 7% of exon positions but were omitted in whole chromosome analysis for simplicity. Hinge elements were subsequently identified either for the whole chromosome in the read orientation (positive, negative) by mapping in the orientation of the read-frame (exon-junk) such that their size was below the predicted threshold either 200 bp (1 nucleosome hinge) or 500 bp (2.5 nucleosome hinge). The length of transcribed (coding) and non-transcribed (junk) sequences were summed in the intervening segments. The equations above were compared as described for the observed exon/intron ratio verses gene length, exon vs intron, and coding vs junk in the respective figures. Randomization of only exons occurred by utilizing the generated lengths of exons and randomly repositioning these segments throughout. Randomization of an exon with its associated intron occurred by random permutation of the position of the shared elements along the generated lists for each chromosome. The custom software is uploaded with the accompanying RefSeq files necessary to generate the findings enclosed at Dryad.

### Chromatin Connectivity, Enhancer, and Epigenetic Modifications

The respective data for High-throughput Chromatin Conformation Capture (Hi-C/Micro-C), Chromatin interaction analysis with paired end tag (ChiaPET), ChIP-Seq, and RNA-Sequencing Analysis (RNA-Seq) were all obtained from ENCODE. The respective bed and bedpe files used were uploaded. The analysis for composition of loops, TADs, and ChiaPET loops were described as above where the total content in both orientations were considered for each chromosome. For analysis of element overlap with hinge positions, the identified 200 bp hinges were used and the likelihood of any portion overlapping with a histone modification peak or enhancer peak was calculated. As a control variation in the read lengths covered by each element, the hinges observed when exons were randomly scrambled across the chromosome length were used. A two-tailed t-test comparing the observed frequency of overlap of the feature with a hinge per chromosome was compared to the observed frequency in the randomly generated hinges.

### Chromosome Paint

Human myoblasts were differentiated into myoblasts and chromosome painting was performed on myotubes as described previously^{33, 46}. Images were acquired on a Nikon Confocal Microscope or a Zeiss LSM 800 Confocal microscope. Imaging was done with a Plan Apo VC 100×1.4 NA oil objective as a multidimensional z-stack. The acquired 3D image stacks were then fed through imaging processing pipelines utilizing standard tools on Cell Profiler and Fiji pipelines performed the functions of translating images to maximal projections, calculating distance, object size, nuclear size, and radius^{94, 95}

### Introduction

As noted above, there is broad consensus that the basic functional assembly of the 2 meters of human DNA is as beads-on-a-string nucleosome networks, observed on ChromEM imaging in both high- and low-density regions of the nucleus.¹⁻³. Above this length scale, chromatin assembles into packing domains that were resolved through the development of ChromSTEM tomography.²⁻⁵. PDs are power-law, mass-fractal geometric assemblies (mass-density scaling within the radius occupied) that are distinct from topologically associated domains (TADs) observed on Hi-C. Structurally, they are composed of 10 Kbp to ˜2 Mbp of DNA spanning a radius between 10-200 nm. PD formation is co-dependent on transcription and loop extrusion: generating long-range connections from enhancer-promoter, promoter-promoter, and cohesin-loops produces nascent (small, poorly packed domains) PDs. Efficiently packing DNA into PDs (maturation) results from charge stabilization by multivalent ions and excluded volume producing geometric specificity for chromatin remodeling enzymes (i.e. small heterochromatin enzymes preferentially localize to the interior). Functionally, mature PDs are a cohesive, geometrically defined unit with three zones: a high-density core, an intermediate-density ‘ideal reaction zone’, and a low-density outer zone. This ‘ideal reaction zone’ or ‘ideal zone’ is relevant to gene transcription: it balances the benefit of local density through the entropic gain of forming intermediate complexes with the decreased diffusibility of the reactant species.^{2, 6-8}. The high-density ‘core’ creates stability while optimizing space in a crowded nucleus and the ‘outer zone’ provides accessible binding sites to transcription factors. The twofold consequence of this geometry resembles a physically encoded ‘transcriptional memory’: (1) transcription shapes and defines the composition of PDs by forming nascent domains, while (2) high-density, heterochromatic regions sustain optimal transcription by creating favorable physiochemical conditions along an ‘ideal zone’ surface. These encoded memories can be sustained for prolonged periods due to geometric specificity of chromatin remodeling enzymes, providing the mechanism for post-mitotic cells to sustain durable patterns of transcription even after the initial signal dissipates. In replicative cells, this geometric specificity is likely projected as 1-D epigenetic modifications heritable through division cycles.

However, the coupling between transcription and heterochromatin into a single functional geometric unit creates a paradox from the perspective of a gene body. Where precisely is heterochromatin deposited to optimize the transcription of a gene without sterically inhibiting the transcriptional machinery? If the entirety of a large gene is always transcribed, how does the polymerase deal with high density repressive marks placed within domain cores? Experimentally across tissues, large genes appear to contain domain cores (H3K9me3 or H3K27me3) within intronic and adjacent intergenic segments as transcription proceeds within adjacent exons (FIG. 18A) mirroring a surface area-to-volume assembly (SA/V). Does this positioning indicate that intergenic segments between genes can structurally function like introns? Could this shared structural property explain the emergence of read-through pathological events in diseases of aging^{9, 10}? Further, how does this system account for gene overlap in anti-parallel orientations?10 Do genes non-randomly generate domain geometries primarily in the orientation of the reading frame? Overall, these questions converge on a fundamental physical problem in the human nucleus: is there a genetically encoded solution in the genome that translates into the SA/V necessary for transcriptional reactions to efficiently occur within the manifested packing domains?

Given the findings in gene bodies across tissues (FIG. 18A), it was expected that non-transcribed ‘junk’ segments (introns/intergenic DNA) non-randomly pairs with adjacent exons to provide the encoded solution to these SA/V problems¹¹⁻¹⁵. These segments predictably translate into geometrically integrated units in the orientation of the reading frame from the linear sequence. For this hypothesis to be valid: (1) there would be a length-dependent pairing of non-transcribed junk segments with adjacent exons to generate SA/V features, and (2) this pairing will depend on the orientation of gene transcription. This would indicate that gene length, gene spacing, and the pairing of intergenic/intronic segments with exons is non-random in human cells. Within this work, this hypothesis was tested, showing that exons are geometrically coupled to their adjacent non-transcribed junk segment to form power-law SA/V assemblies oriented with the transcriptional reading frame. Whether this is a general solution to efficiently pack DNA into the eukaryotic nucleus or an evolutionary mechanism to generate long-lasting but modifiable PD structures was then explored. The SA/V genomic organization was absent in S. cerevisiae, whose much smaller genome organizes into nearly linear compositions in both genes and chromosomes. Instead, gradual transitioning from a linear to power-law genome in complex, multicellular organisms was observed. Collectively, this indicates a physically encoded ‘geometric code’ linking transcribed and adjacent non-transcribed units into cohesive physical structures. This geometric code immediately generates new avenues of research across diseases of aging, species evolution, RNA transcript type, and complex organ development rooted in physical genomics.

### Results

### Genes and Loops in a Crowded Nucleus

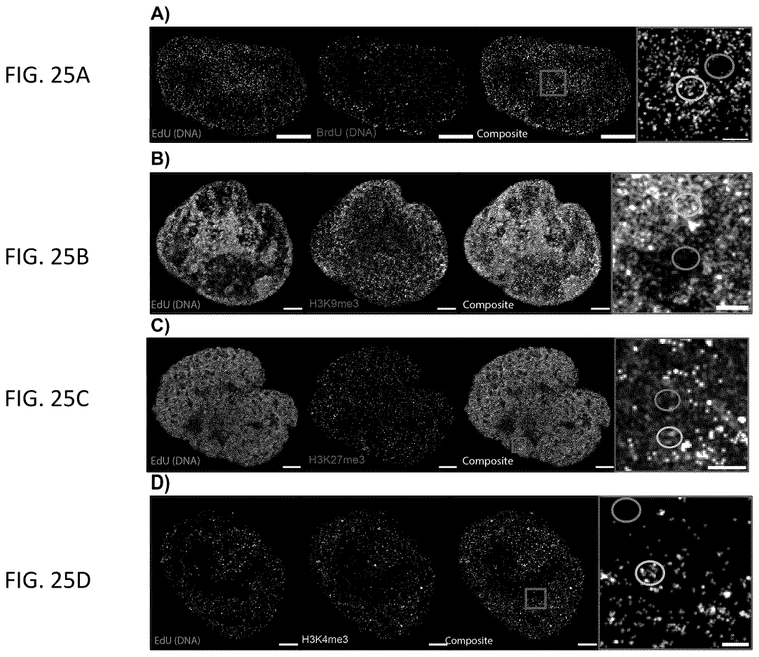

There are numerous sequence-specific genomic regulatory elements of transcription: enhancers, promoters, insulators, and silencers, among others¹⁶⁻¹⁹. Their sequence compositions are widely studied in human disease and form the basis of much of our understanding of the regulation of gene expression. They are particularly suited for study by proximity capture methods (Hi-C, Sprite, etc)²⁰⁻²² and in situ hybridization imaging²³⁻²⁵ as a long-range connection is a deviation from how a polymer, such as chromatin, would otherwise expand in space. For structural studies of chromatin, super-resolution imaging is frequently employed to study the nanoscale structure of the genome in situ^{2, 4, 26-33}. However, reconstructions and analysis using super-resolution methods is fundamentally dependent on the ability to compare the observed reconstructed structure to findings on electron microscopy. Therefore, assumptions in super-resolution imaging of chromatin can conceal the ground-truth structure if they are not grounded in ChromSTEM or ChromEM. For example, the size of antibodies alone produces a non-monotonic label density as a function of DNA density. The result from the steric hinderance of antibodies is that both high-density and low-density regions could appear devoid of signal in super-resolution imaging (FIGS. 25a-d)^{15, 34}. While hybridization would represent an ideal solution, formamide denaturation for sequence-specific fluorescent in situ hybridization (FISH) imaging collapses nanoscale domain structure³⁵⁻³⁷. While FISH reliably identifies strong long-range genomic connections, formamide destabilization means it is poorly suited to provide nanoscale geometric information of chromatin³⁷. Collectively, this indicates that only recently were the tools (via ChromEM and ChromSTEM) available to accurately probe questions pertaining to nanoscale human genomic geometry.

As a result of the previously unknown limitations of these imaging methods, we may know very little about how to translate the linear genome into the physical geometries observed on ChromEM/ChromSTEM imaging^{2, 3}. To link the linear and 3-D sequence space with cell function, the focus of gene transcription was first expanded to include both the physical and informational properties of genes themselves in the process of gene transcription. Transcription is a series of diffusion-limited chemical reactions⁶⁻⁸. The reactant species include transcription factors, polymerases, and DNA, among many others, with the output product a coherent segment of RNA^{2, 6-8, 38} Therefore, independent of the type of RNA product, microRNA (miRNA), long noncoding RNA (lncRNA), and protein-coding messenger RNA (mRNA), among many others, the DNA sequence producing these molecules contains sequence-dependent information and occupies physical space within the nucleus (FIG. 18B). In the orientation of the reading frame and from the perspective of the stored sequence information, DNA transcribed and processed into a functional RNA is referred to as ‘exons’ and the DNA that is infrequently transcribed and processed as introns (within a gene body) and intergenic segments (between gene bodies) (FIG. 18B). Collectively, the colloquial term ‘junk DNA’ is used when referring to both introns and intergenic segments to demarcate DNA not generally transcribed into an RNA product¹¹⁻¹³. There are many additional regulatory systems stored within different types of produced RNA 39-41 However, a focus here is testing whether there is a predictable translation from the 3-D geometry observed on electron microscopy into the linear chromatin polymer encoding information for efficient transcriptional reactions. For simplicity, a two-element schema of coding segments (exons) and non-coding segments (junk) are retained in this disclosure¹¹⁻¹³. Finally, no assumptions are made about sequence specificity differentiating between exon and junk segments with chromatin remodeling enzymes, instead focusing on the physical assemblies in the context of 3-D geometry and transcription reactions.

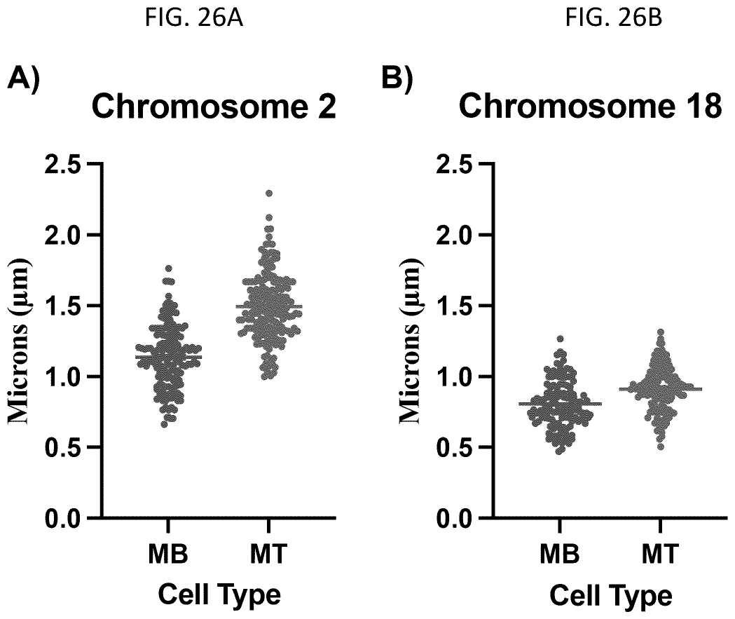

To translate DNA segments into space-occupying physical structures, how DNA organizes into the beads-on-a-string of disordered nucleosomes and then into higher physical assemblies is considered^{1-3, 26, 27, 42}. There are several histone protein and nucleosome variants within human cells. Despite these variations, the sequence length within a monomer typically ranges from 130 to 160 bp with a linker segment of up to 80 bp⁴³⁻⁴⁵. Likewise, nucleosomes potentially slide along the linear segment for ˜100 bp. As a result, somewhere between 130 bp to ˜300 bp is a reasonable discretization of sequence-length into a space-filling unit. For illustrative purposes and consistency, 200 bp and myosin heavy chain 1 (Myh1) are selected as a representative example. Myh1 is composed of ˜26,000 base pairs, which translates into a linear assembly of ˜130 nucleosomes. A nucleosome is approximately a cylinder, with diameter of ˜11 nm and a height of ˜5.5 nm. Stacked end-to-end, Myh1 would be a ˜715 nm loop or rod with a diameter of 11 nm. For comparison, chromosome 2 is composed of 243 Mbp and in mature muscle cells has a radius of ˜1.5 μm (FIGS. 26a-26b) 46. Extended further, an enhancer-promoter loop that is 100 kbp would be a 5.5 μm loop that roughly spans the radius of the nucleus if not folded in space (FIG. 18C)^{47, 48} This state is rarely observed experimentally⁴⁷⁻⁴⁹. Accounting for the efficient assembly of genes uses a different geometry. While the exact configuration of Myh1 (and all other genes), is unknown in every cell in all conditions, the measured physical structure of the human genome at length scales between 10 kbp and ˜2 mbp is that of a mass-fractal observed as packing domains on ChromSTEM. Above these scales, it behaves as a territorial polymer that doesn't depend on the underlying structures. This indicates that genes, especially large genes >5 kbp such as Myh1, are better described by a mass-fractal arrangement, where the nucleosomes occupy the space in a power-law manner^{2-5, 15, 50, 51} (FIG. 18C). Above these scales, at the micrometer length-scale, chromatin geometry is not defined by the structure of the domains³.

If large genes such as Myh1 are mass-fractal structures, their geometry can be statistically described by how the monomer (˜a nucleosome) spans the occupied volume as linked segments by its polymer structure (FIG. 18C). This property is defined by the mass (nucleosomes) scaling as a function of radius to the dimension, D, by the equation Mass (nucleosomes)=r^{D} which was confirmed on ChromSTEM tomography^{2, 3, 52, 53}. There are two limiting cases of mass-fractals in chromatin that are worth considering for illustrative purposes. A random walk without any attractive potential or confinement will produce a mass-fractal with scaling of D=2, which has a uniform distribution of density within a typical domain volume (FIG. 18d). An extension of this is a confined random walk, the fractal globule, which will have a contact scaling of D=3 (FIG. 18e)⁵³. This configuration again has a uniform distribution of density, but the volume is fully filled. In living human cells and in ChromSTEM resolved packing domains, D values are observed to range between 2.2 and 2.8, indicating non-uniformly distributed density with a radial decay from high-density interiors to low-density periphery^{2, 3}. This density gradient pairs the high-density (heterochromatin) interiors with lower density (euchromatin) into a single functional unit to generate transcriptional memory (FIGS. 18b, 18c, 18f). This occurs because the density gradient creates geometric specificity for enzymes due to their size and transcription defines the components of the segment^{26, 34}. However, this creates a regulatory paradox in the gene body since RNA polymerases would seemingly ‘pass through’ a high-density heterochromatin segment. This occurs if one were to assume that transcription of a gene occurs in its entirety through the gene body, including all the intron segments. However, recent work in has demonstrated that the rate of observed splicing can greatly exceed the rate of transcription for long introns, suggesting that most of the sequence of large introns is bypassed⁵⁴⁻⁵⁶. The result is that this paradox can be resolved by 3-D organization if these reactions are occurring along an ideal zone ‘surface’ (FIG. 18b).

### Human Genes are a Predictable, Power-Law Geometric Assembly of Exons and Introns

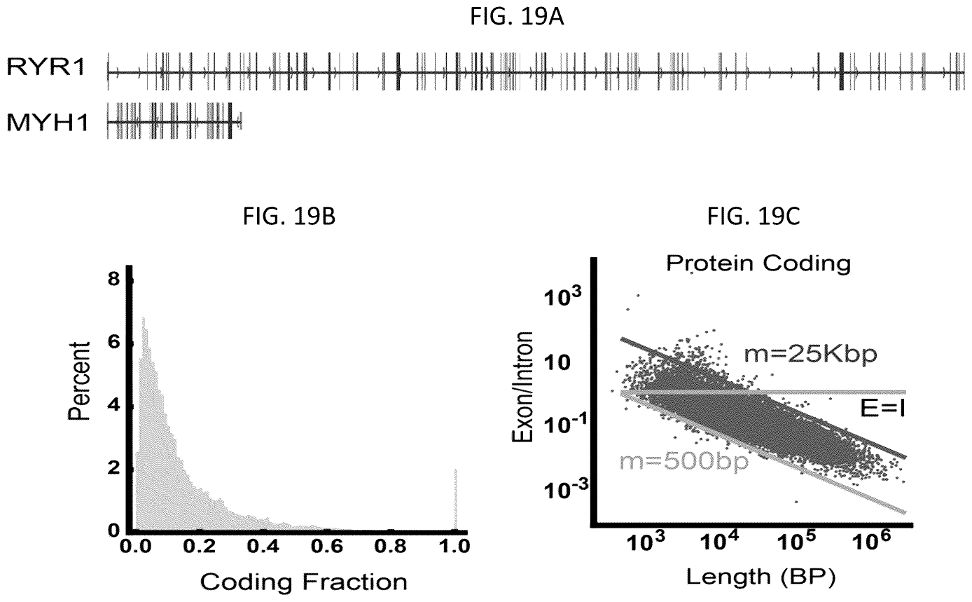

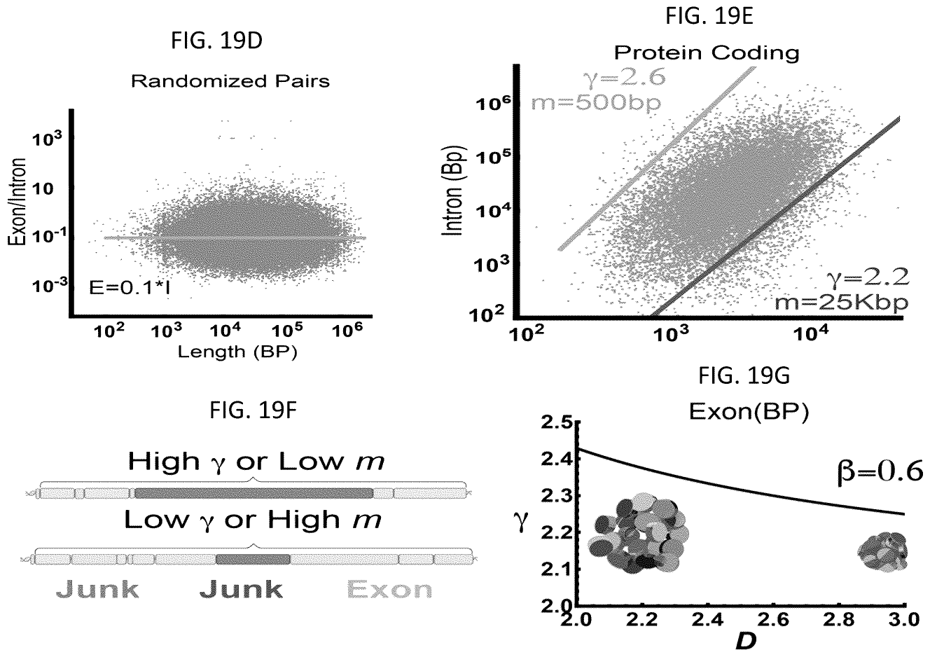

Organizing genes into power-law geometric domains has functional benefits: space is optimized, enzyme positioning is geometrically encoded, and transcriptional reactions are stabilized. In essence, this organization generates the SA/V ratios necessary for reactions to efficiently occur in the crowded nucleus. What is missing is whether this becomes imprinted as a geometric code stored within the linear genome. Since 98% of the human genome is composed of infrequently transcribed ‘junk’ DNA, this is an ideal candidate for the storage of density information to produce the SA/V of PDs. It was expected that if most human genes use power-law geometry for improved function, then an inverse relationship between the proportion transcribed (exons) exists relative to space-organizing segments (introns) as a function of the gene length. This is akin to the inverse relationship between SA/V and the radius of a sphere, but dependent on the translation of a polymer into a volume. This inverse relationship would indicate that intron size increases as a power-law of exon length to position the coding fraction to the ideal zone. This would imprint an SA/V relationship encoded in the linear segment lengths (FIG. 18c). The result is a critical function of non-coding segments divorced from the nucleotide composition to effectively fill the occupied space as a mass-fractal PD to correctly position coding segments in the path of the active polymerase. An illustrative example is a comparison between Myh1 and Ryr1. Both genes are crucial to muscle function but are not transcription factors directly, with very distinct functions. Human Myh1 is composed of ˜6,000 bp of exon sequences and ˜20,000 bp of non-coding sequences. In contrast, Ryr1 is composed of 15,000 bp of exon sequences (FIG. 19a). If exon length is not related to intron length, one would reasonably assume that exon length would be ˜2% of any gene body. This is because only 2% of the whole genome is exons. Instead, there is a broad distribution in the exon fraction within genes, with a median of 9.8% (FIG. 19b, interquartile range of 4.6% to 21.1%). An alternative possibility is that intron length linearly increases with the exon length to facilitate bending of the synthesized RNA for splicing. In this case, one would observe the intron composition to be ˜60,000 bp in Ryr1 (˜3× as long as that of Myh1). Instead, and consistent with a power-law geometry for a space-filling function of introns to facilitate positioning of transcribed segments to the ideal zone, the total intron length of Ryr1 is 145,000 bp.

To test the hypothesis that exon length is related to intron length by this geometric power-law relationship, the publicly available human reference genome GRCh38 was used, with annotations from RefSeq.⁵⁷ The ratio of exons to introns vs total length was calculated for all protein-coding genes and found that much of the human genome is described by this geometric principle (FIG. 19c, Example 2 Materials and Methods). This relationship is defined as follows by Equation (1) where m is a scalar in basepairs:

$$\frac{E x o n}{Intron} ≅ \frac{m}{{Length}^{n}} . \qquad 1 )$$

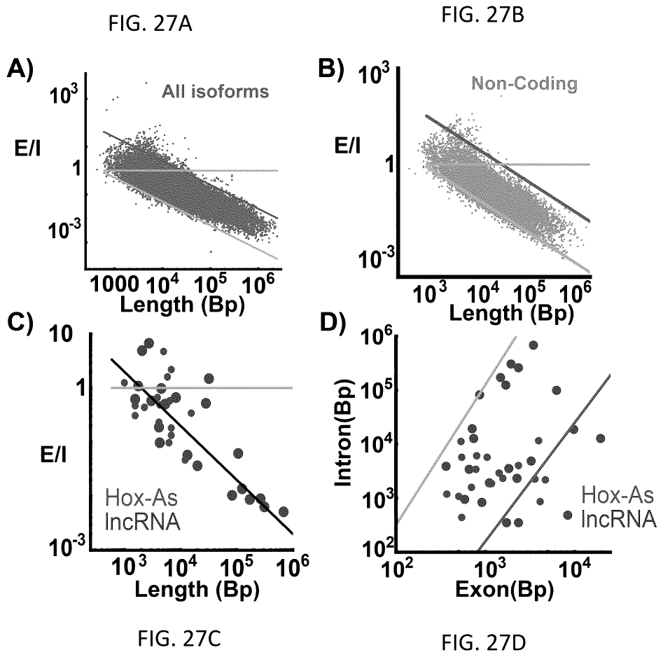

Within the human genome, m ranges from 500 bp to 25,000 bp, which would be consistent with ˜2 to 125 nucleosomes and n is close to 1. This is observed both using a single isoform or all the variant isoforms within the RefSeq database (FIGS. 27a-d). An alternative explanation for such a trend is that as gene length increases, the fraction that is coding will decrease. This is less likely since a more reasonable, statistically grounded null hypothesis is that the ratio of exon to gene length would be randomly distributed around the constant fraction observed throughout whole chromosomes. However, given this consideration, whether gene length is geometrically related to the exon length was next explored. First, the exon to intron (E/I) with gene length for all coding isoforms was randomly redistributed, which produced a size-independent constant ratio of ˜0.1 (FIG. 19d). Next, if exon length defines the intron length for domain formation by an SA/V-like relationship, intron length would be a power-law of exon length. The values of the exponent will range between 2 and 3 related to the conversion of the linear chain into the power-law volumes observed in imaging. Validating this hypothesis, this relationship (FIG. 19e) was observed. This is captured by the relationship described in Equation (2) where m again ranges from 500 bp to 25,000 bp:

$$Intron ∝ E x o {n}^{γ} / m . \qquad 2 )$$

Understanding the function of the scalar, m, and the exponent, γ, requires considering how these translate information from the linear chain into 3-D geometry. Along the linear genome, m and γ control the density of coding information, with a larger m value (˜25000 bp) and lower γ(˜2) producing less spacing to generate the desired configuration. In contrast, a smaller m (˜500 bp) and larger γ(˜3) produce more linear spacing to achieve the effective 3D configurations. However, it is possible that genes utilize both degrees of freedom to determine how to translate into an effective configuration. A high m and lower γ state has high information density. As a result, structural genes such as Myh1 (m˜7800, γ=2.2) adopt more complex geometries. The variable γ is inversely proportional to D as the transformation of the chain along the occupied volume, quantified by

$$γ = 1 + \frac{D}{D - β} , \qquad 3 )$$

where γ is the fraction of exon positioned within the ideal zone (Ex 2 Materials and Methods). As a result, genes with low γ states will adopt a higher-D configuration for optimal function, in essence necessitating the deposition of high-density chromatin within the chain segment that translates into the volume (FIG. 19f).
Power-Law Geometry is Associated with Organo-Axial Development

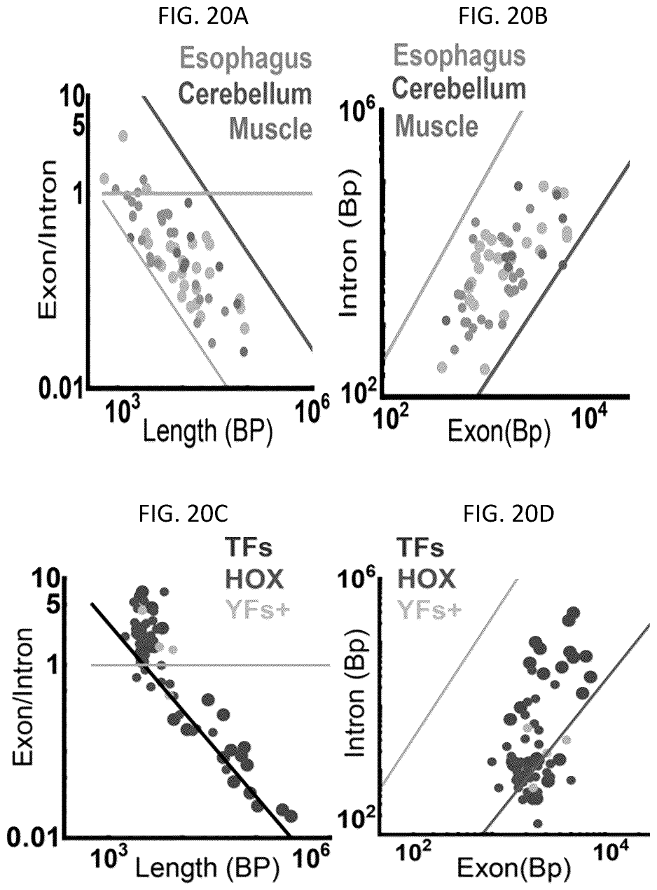

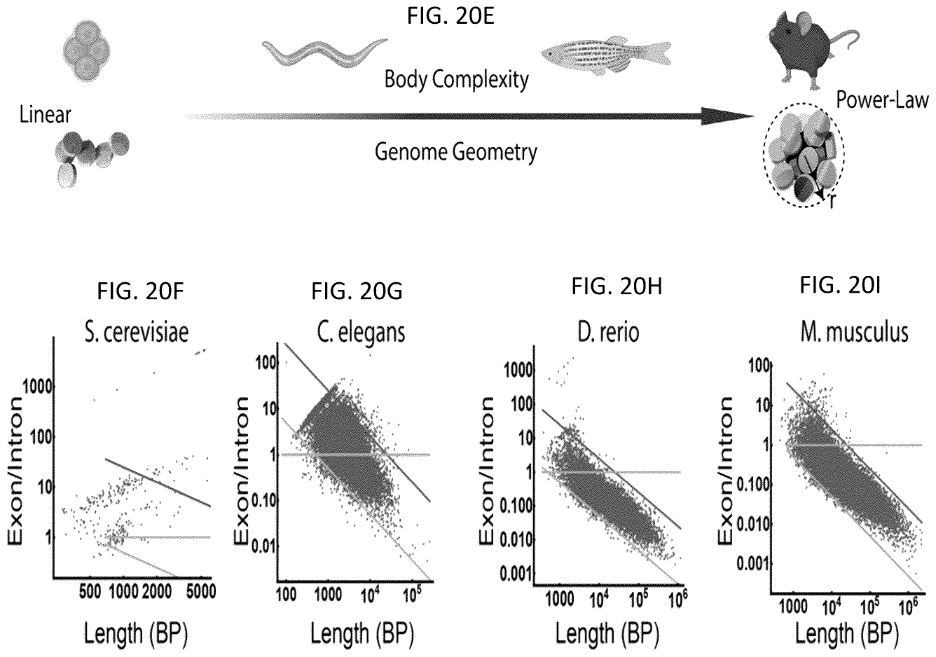

These relationships indicate that this geometry is conserved across the human genome. Therefore, whether this simply reflects the process of splicing, or a novel regulatory framework based on physical interactions was considered. Two null hypotheses were considered: (1) this geometry lacks any relationship to cell function and (2) it is merely an unintended consequence of RNA splicing. To test if this encodes a function, differentiated tissues from each germ layer were analyzed: esophageal mucosa (endoderm), cardiac muscle (mesoderm), and cortical neurons (ectoderm). GTEx expression data from these three sites was utilized and genes selected that were preferentially associated with that tissue (Data not shown)⁵⁸. Strikingly, across these diverse tissues, the conservation of this power-law geometry for genes involved in maintaining tissue function across the human life-span was observed (FIGS. 20a and 20b). Next, transcription factors were randomly selected across functional families given the degree of complexity of transcription factor networks in determining organo-axial development (Data not shown) and compared their behavior with Yamanaka factors and HOX genes.^{59, 60}. Interestingly, most Yamanaka factors and HOX genes deviated away from this power-law organization (FIGS. 20c and 20d). In Yamanaka factors and HOX genes, the observed exon to intron ratio appeared to favor synthesis of exon segments over production of introns (E/I>1). Despite this general deviation, a few HOX genes strongly associated with a geometric domain composition (i.e. intron length is a power-law of exon length). Therefore, the behavior of transcription factors was analyzed in relation to developmental timing and found that short genes involved in early development (pluripotency factors) tended to favor exon enrichment whereas genes involved in end-organ development (e.g. MITF61 or RUNX262) were associated with power-law composition (FIGS. 20c and 20d). Collectively, this indicated at least two complementary systems encoded of transcription factor programs: primordial factors favoring the linear density of information and tissue-specific factors favoring complex assemblies.

Next, comparative genomics was utilized to test if this organization is an unintended consequence of RNA splicing or a novel regulatory framework associated with complexity. Since genes involved in complex tissues functions are organized as power-law assemblies (FIGS. 20a-d), it was expected that the power-law composition of genes is beneficial for organoaxial development. Specifically, post-mitotic cycle cells (neurons, muscle) can maintain function for extended periods by physically encoding domain structure within the genes essential to their long term function. While developmental cues can activate these domains, once they are in place they can be sustained by geometric specificity²⁶. If this were the case, power-law compositions would ‘emerge’ with the presence of body plan complexity and not with splicing itself. In organisms that rely on splicing for complexity (short splicing segments) instead of domains (variations in length), genes containing more exon than intron (E/I>1) were expected. Therefore, whether the observed phenomenon was directly the result of the existence of RNA splicing, or a method of functional organization that utilized it was tested. Comparative analysis across eukaryotic genomes capable of alternative splicing was performed, the following were specifically chosen for analysis due to their well characterized genomes and the existence of nucleosomes, introns, and splicing machinery: S. cerevisiae (a model of monocellular organism with splicing and introns), C. elegans (multi-cellular organism with simple organoaxial positioning), D. melanogaster and D. rerio (multi-cellular organisms with complex organoaxial positioning), and M. musculus (non-primate mammal with complex organoaxial positioning)⁶³. Consistent with the hypothesis that geometric encoding could facilitate organ complexity, power-law assembly of exons with introns emerged with organ specification, transitioning in parallel with developmental complexity (FIGS. 20E-20I). The transformation from exon enrichment (S. cerevisiae) was observed first toward a power-law composition where exons outweigh introns (C. elegans). This transition toward a power law interestingly occurred as body plan complexity increased, suggesting an evolutionary benefit for producing geometric complexity (FIGS. 20E-20I). Overall, this transition indicated that the power-law composition of genes built upon splicing, introns, and nucleosomes, but was not merely a secondary product of splicing activity.

### Non-Protein Coding RNA Genes are Power-Law Structures

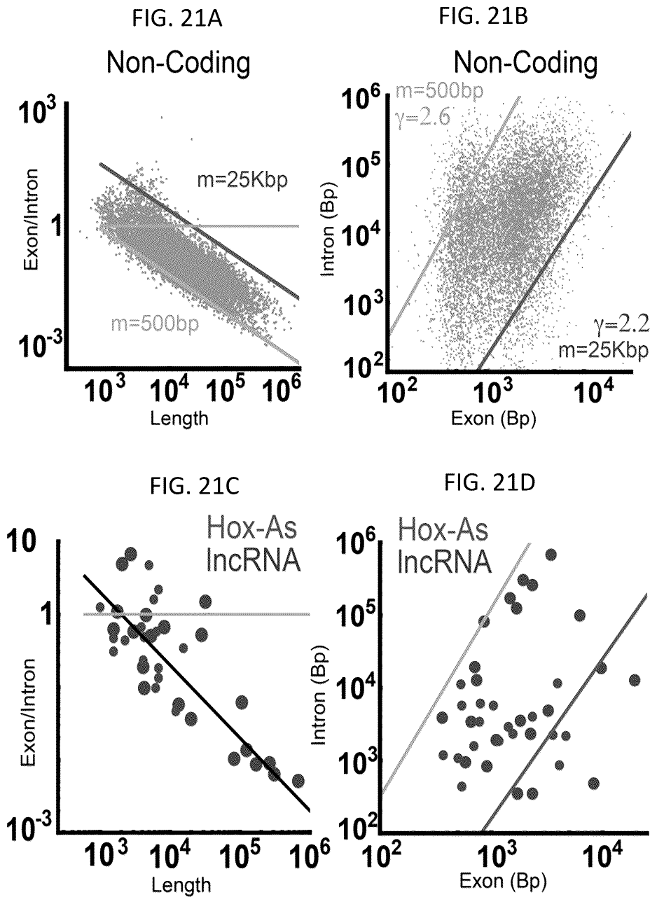

Next, the hypothesis that this geometric assembly intersects with the physical principles needed for efficient transcription reactions independent of the RNA product (mRNA, miRNA, lncRNA, pseudogenes, etc) was tested. If this observation was indeed rooted in the physical principles of SA/V and the chemical reactions of transcription, it would generalize across transcript type. If true, this finding would make the chemical process of transcription reactions themselves intertwined with and central to power-law domain geometry within the human genome. Remarkably, power-law composition of non-protein coding DNA segments across the human genome (FIGS. 21a-c) was again observed with identical constraints of m and D as protein-coding genes. A subset of long-noncoding RNAs associated with the HOX clusters in humans and in tissue development was again investigated^{64, 65}. Interestingly, the lncRNAs associated with HOX genes in humans displayed a comparable organizational structure to that observed in HOX genes that encode for proteins (FIG. 21D). Likewise, long-noncoding RNAs that were involved in tissue formation and development had similar behavior to protein coding genes involved in terminal differentiation (FIG. 21D). A subset of these lncRNA involved in stemness across multiple tissues were organized like Yamanaka factors (e.g. H19)⁶⁴⁻⁶⁶. Collectively, these findings indicated transcription is intrinsically coupled with the power-law geometric organization of the human genome, and not the function of the final product (i.e. proteins or non-coding RNA).

### The Non-Random Power-Law Geometric Assembly of Human Chromosomes

Since human genes organize into power-law compositions independent of RNA product, it was expected that human chromosomes would organize as coherent assemblies of domains. From the physical perspective, intergenic spaces would have similar structural properties as introns. Collectively, if this were true, exons would be paired with junk (introns, intergenic regions) similar to the organization of introns within individual genes. For such an organization to exist and interact with transcription, it necessitates positioning of coding segments across genes non-randomly with junk segment as a power-law in the orientation of the reading frame would be necessary. This relationship would indicate that, in general, regularly transcribed human coding material uses enough non-coding material to support its 3-D geometry. This finding would have a profound evolutionary implication on the adjacent positioning of genes. It would indicate that length and spacing, and not the sequences alone, can be under selective pressure. Further, gene and exon position would not be a random event but is instead driven by physical and chemical principles.

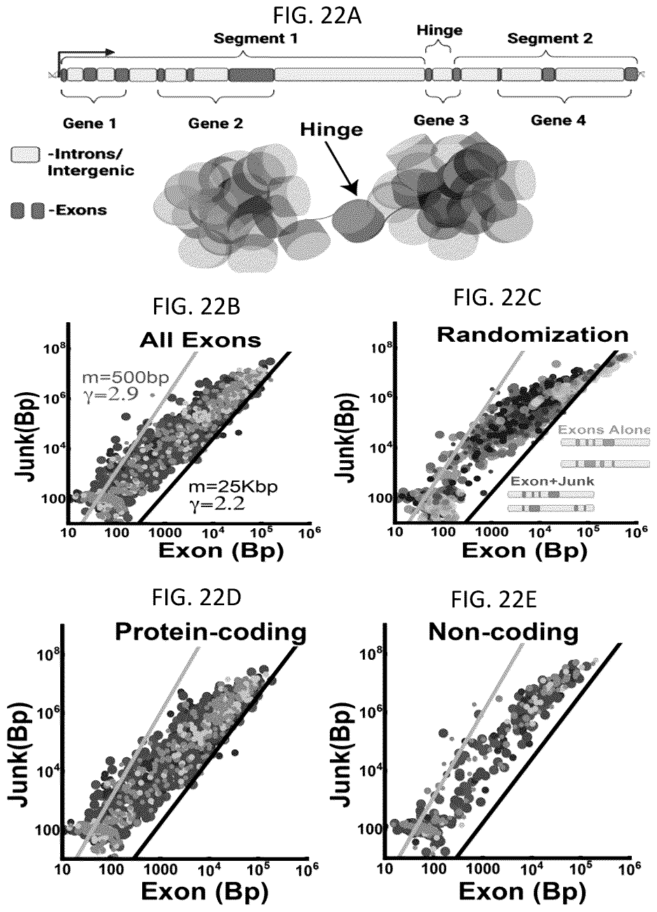

On both ChromSTEM imaging and in polymer simulations, packing domains were observed to be separated by an interdomain space²⁻⁵. This indicated the existence of linker or hinge element within the physical chain to separate packing domains in space. Without the presence of a hinge spacer, domains become superimposed in 3D space and no inter-domain space was present to accommodate passage of non-chromatin bound macromolecules such as RNA (FIG. 22A). As the basic-structure of the genome is nucleosomes organized as beads-on-a-string, it was expected that hinge elements would represent bifurcations that contained a coding segment (for transcription specificity) and a non-coding segment to correctly fill the 3-D space. If hinges were instead composed of only coding segments, the supercoiling generated by polymerase could potentially alter the composition of the hinge segment^{67, 68}. Alternatively, if the hinge were completely non-coding, there would be no functional specification to delineate segments in a transcriptionally specified manner. At minimum, one would anticipate that a hinge element would therefore be around the composition of one to a few nucleosomes in length (˜140-500 bp). Such segmentation effectively separates between domains that could range between 10-30 nm (if the hinge is completely collapsed into nucleosomes) or to ˜100 nm of stretched DNA purely from the length of uncoiled DNA. At maximum, it should not span more than several nucleosomes (˜1000 bp) in length, or it would begin to interact with nucleosome remodeling complexes like domains via geometric specificity. As a result, this provides a dynamic range across cell types and biomechanical forces exerted on the cell nucleus rooted in physical observations of chromatin.

Validating the central hypothesis, the immediate consequence of accounting for spacing between domains (hinge bifurcation) was the organization of every human chromosome into power-law SA/V segments of transcribed/junk-units in the reading orientation (FIGS. 22B-C). Further the organization in the human genome across chromosomes reflects the same principles as in individual genes as described by Eq. 3 with m and γ adopting the same values and meaning:

$$Junk ∝ {Exon}^{γ} / m . \qquad 4 )$$

To test if this occurs from transcription alone (coding elements) or an intrinsic pairing of coding—/junk—units together, the coding elements were randomly distributed alone or as a paired element (coding+junk). With the permutation of coding element position alone, only hinge like positions and large-scale (>several MBP) segments occur. Indeed, domain length scale compositions are completely lost in both strand orientations (FIG. 22d). Consistent with an intrinsic coupling between adjacent coding-/junk-segments oriented in the direction of RNA polymerase, power-law assembly is conserved when the coding-/junk-units are permuted together (FIG. 22e). This finding indicates a physical coupling between a coding segment with its associated junk segment in the read orientation to produce SA/V assemblies (FIGS. 22d-22e). Interestingly, the information stored in paired coding-/junk units is not delineated by gene boundaries, but instead that segmentation is intended to support each reading segment with a space-filling unit. If this process is intrinsically linked to transcription and not the end product, it should be preserved with either protein coding or non-coding segmentation. Independent of the type of RNA generated (protein coding RNA, non-protein coding RNA), chromosomes assemble into power-law segments of coding-/junk-elements (FIG. 22f). Collectively, this indicated that chromosome assembly is defined by the act of transcription itself reflected in the physical properties observed in PDs.
Chromosomes Transformed from Linear to Power-Law Geometric Assemblies

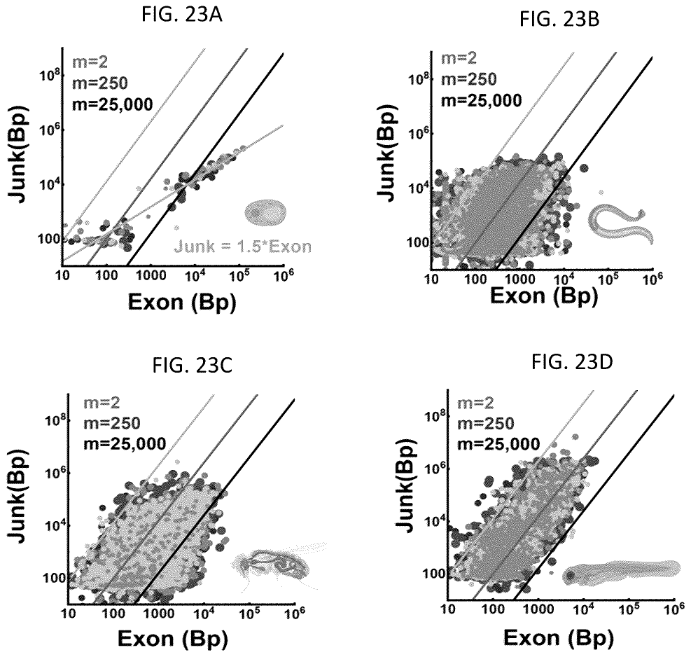

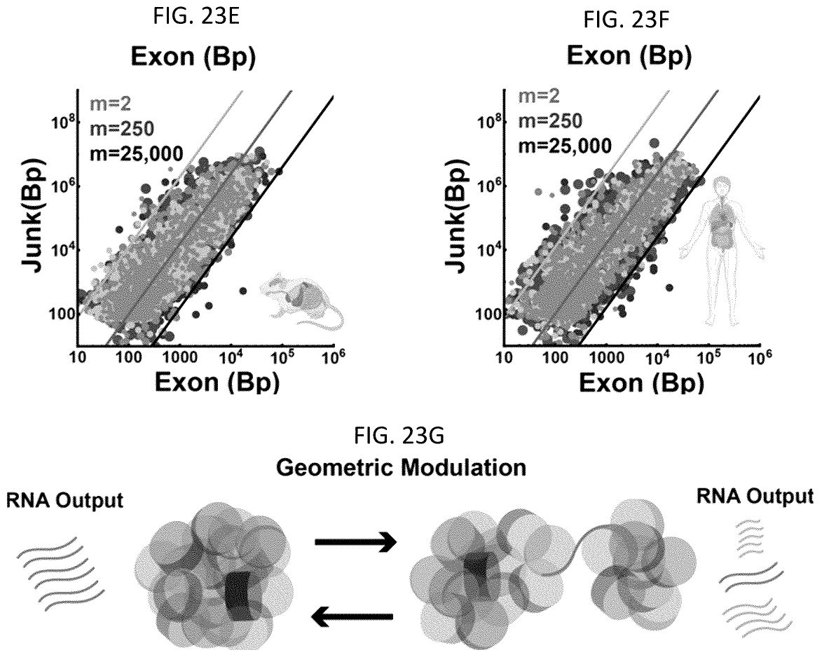

Given these findings demonstrating a transformation of genes in eukaryotes in association with the development of body plan complexity, it was expected that chromosomes should transition in a similar manner. This would imply that the linear positioning of genes in the orientation of the reading frame is an evolutionary property for the reasons described above. The RefSeq genomes⁵⁷ for respective organisms across eukaryotic complexity were utilized and their chromosomes with hinge segmentation segmented. Consistent with transformation of genes, chromosomes were observed to be linear assemblies in S. cerevisiae (junk˜exon) (FIG. 23a) in both read orientations. This indicated that the human genome, which organizes as a power-law, organizes by fundamentally distinct physical principles. Chromosomes in eukaryotes were next examined as a function of their body plan complexity from C. elegans to mice. Consistent with the observation of gene transformation into power-law compositions with the progression of complexity, this was observed with chromosomes (FIGS. 23b-e). The transition between drosophila and zebrafish was the most remarkable, with chromosomes transitioning from a mix of linear and power-law pairs into mainly a power-law structure (FIGS. 23b-c). As expected, the genome with the most similar geometry to the human genome was that of the mouse, which organized into comparable partitions of coding/junk pairs (FIGS. 23e and 23f). From the perspective of SA/V in the S. cerevisiae genome, if domains still form, it needs to come at the expense of the neighboring genes. An alternative is that transcription in S. cerevisiae instead occurs in between domains along the linear space connecting domains. More likely based on prior literature demonstrating that accessibility is relatively uniform throughout S. cerevisiae is that genes adopt a generally loose but inefficient configuration. In either case, packed assemblies would behave primarily to produce antagonistic transcription states in the neighboring genes; one gene must be off for another to be on to facilitate packed space. As a result, in 1-D, compaction within S. cerevisiae effectively translates into a 3-D barrier⁶⁹. In contrast, in genomes organized as power-law assemblies (D. melanogaster onward), deposition of heterochromatin marks in introns/intergenic junk conferred the ability to facilitate a geometric modular enhancement to the associated exons by creating and regulating the elements positioned on the reaction surface (FIG. 23g).

TADs and Loops Integrate with but do not Define Power-Law Geometry

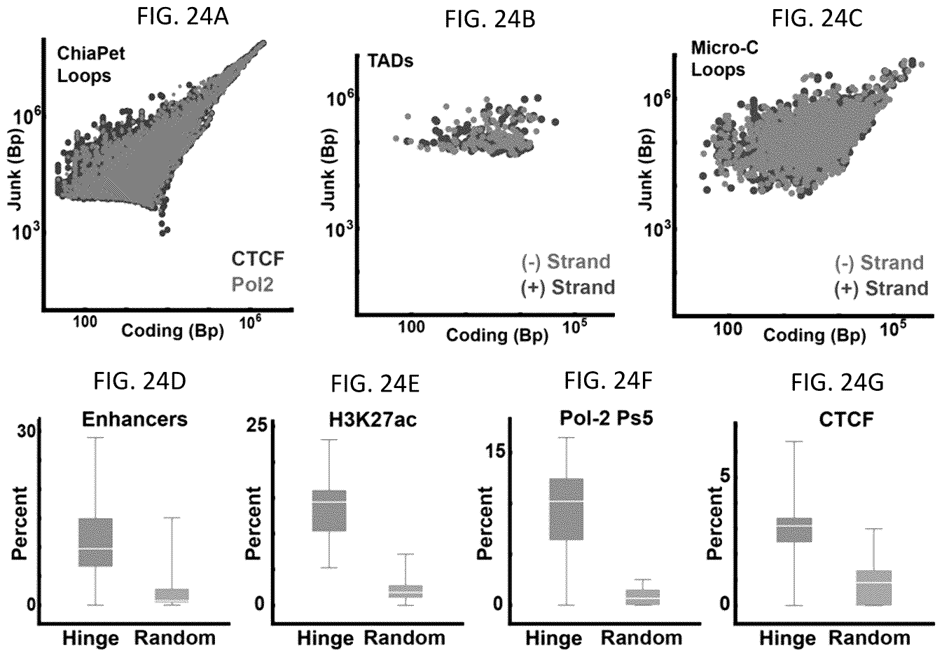

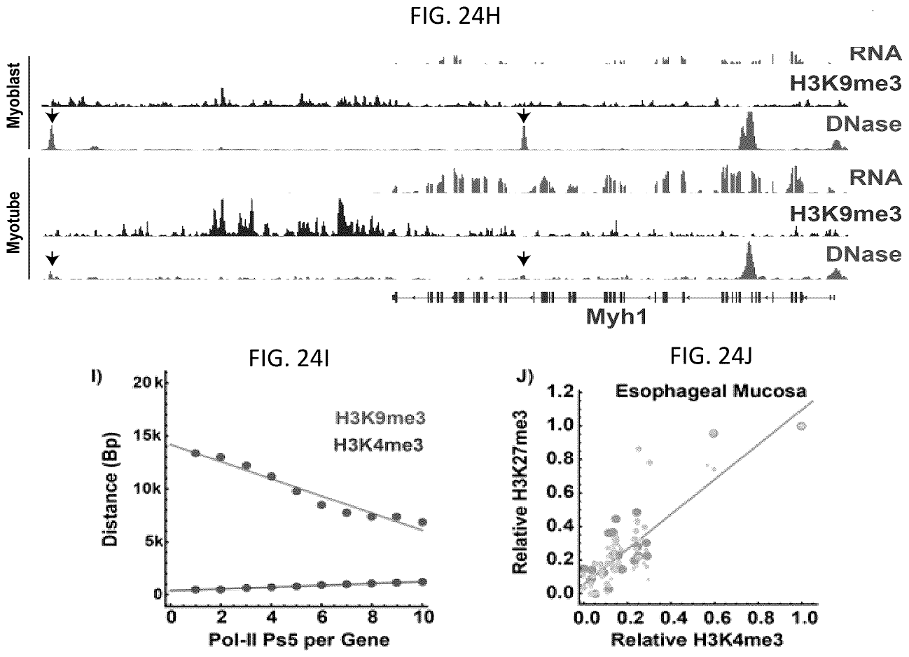

Given the role of connectivity in coordinating chromatin organization, transcription, and development in humans, whether these observations are explained by loops and TADs measured on Micro-C/Hi-C or paired-end tag sequencing (ChiaPET)^{20-22, 70} was next investigated. Publicly available ChiaPET and Hi-C data for HCT-116 cells through ENCODE for both CTCF and RNA polymerase II (Polr2a)⁷¹⁻⁷³ was utilized. Although these are cancer cells, HCT116 cells have a diploid genome, well characterized features including transcription factor binding sites, nucleosome modifications, and elimination systems targeting chromatin remodeling complexes through the auxin-inducible degron system. Since all chromosomes obey power-law segmentation into coding-/junk-units, the analysis is focused on chromosome 17. Consistent with this physical encoding representing a novel regulatory structure and not merely the manifestation of connectivity, the majority of either Pol II or CTCF driven loops represent deviations away from power-law organization (FIG. 24a). Next, a similar analysis was performed on loop domains, TADs, and compartments obtained from Micro-C. As observed with CTCF and Polr2a loops on Chia-PET, both Micro-C loop domains and TADs display a similar behavior (FIGS. 24b-c). Interestingly, A- and B-compartments were both similarly composed of coding/junk elements but were typically at sizes well above that of packing domains (>2 Mbp in length). Further, the composition of compartments was similar to segments preserved by the random permutation of coding segments across the genome, suggesting that they represented a distinct regulatory regime (FIG. 22). In both ChiaPet and Micro-C, a subset of TADs and loops were composed of power-law assemblies of coding-junk elements (FIGS. 24 a-c). These findings indicated that power-law segmentation was not reflected solely in loop events, but instead represented the organizational principles that arise from geometric assembly. These findings were then verified across all chromosome and supported by the very low contact scaling decay behavior within TADs. This suggests that while some loops provide sequence specificity for domain organization, most provide a complementary functional role. Given the limited overlap between connectivity and coding-/junk-compositions, whether a hinge element represented the manifestation of an enhancer or a TAD/loop anchor was next studied. It was expected that hinges would likely be enriched for ‘enhancer’ features as they would demarcate the behavior of adjacent genes into power-law assemblies. Utilizing ENCODE and the Atlas of Enhancers¹⁷, the frequency that these features occurred with 200 bp or smaller hinges in HCT-116 cells was calculated. Consistent with the findings of loops and TADs, only 3% of hinge elements were bound by CTCF compared with a ˜10% occupancy with transcriptionally active RNA polymerase II (Pol2-Ps5) in HCT116 cells (FIGS. 24d-24g). Likewise, even though hinges were enriched for euchromatin nucleosome modifications (H3K27ac ˜15% of hinges, H3K4me3 occupying ˜13% of hinges) only 10% of enhancers in HCT116 cells aligned with a hinge segment (FIGS. 24d-24g). Reciprocally, only 6% of hinge elements were identified to align with enhancer positions. Collectively, this indicated that these sequence-specific functions intersect with the geometric structure of chromosomes as expected in relation to the act of transcription.

This organizational system in human genes, cells, and tissue was investigated. Findings here and in recent work indicate that transcriptional activation is accompanied by a (1) loss of accessibility in introns and intergenic segments even as the transcriptional start site remains open with (2) an accumulation of heterochromatin in the junk segments. At the cell and tissue level, one would anticipate coupling between heterochromatin and euchromatin at a global level and that increased transcriptional activity corresponds to shorter distances to a high density (H3K9me3) heterochromatin core. We recently described the deposition of H3K9me3 core in the gene body and distal intergenic space of skeletal muscle myosin heavy chain genes (Myh1 and Myh2), which are structural proteins necessary for mature muscle cell function. Therefore, changes in accessibility to these loci were examined using publicly DNAse-Seq in myoblasts (immature muscle) compared with differentiated into myotubes (mature muscle). The loss of accessibility in junk segments was observed as transcription is amplified in both genes (FIG. 24h). Next, whether there is evidence of the core formation system within induced pluripotent stem cells using GM23338 cells was tested. As shown, as transcriptional activity within a gene body increased, the distance to the nearest high-density core (H3K9me3) decreased (FIG. 24I) and that heterochromatin (H3K27me3) correlated at a chromosomal level with amount of active RNA polymerase II (FIG. 22). Interestingly, the distance in pluripotent stem cells to the nearest core was considerably smaller than observed in HCT-116 cells and in SK-N-SH neuroblastoma cells (FIG. 22), suggesting that domain size might be linked to cell differentiation states. Whether similar patterns are present in terminally differentiated tissue was then investigated. Utilizing data available through ENCODE, whether the degree of heterochromatin (H3K27me3) correlated with the amount of euchromatin (H3K4me3) was investigated. Across multiple human samples, this indeed is the case (FIG. 24j). When paired with the findings across genes in tissues (FIG. 18) and recent finding of this process occurring in ovarian cancer stem cells, these findings indicate that these organizational principles span multiple different human tissues, cells, and genes representing a generalized framework for chromatin organization in human tissue.

### Discussion

These findings indicate that terms including ‘junk’, ‘intronic’, ‘intergenic’, and ‘noncoding’ DNA may have obscured the functional role of these segments to act as ‘Volumetric DNA’ in complex, multicellular eukaryotes¹¹⁻¹³. This is likely due to assigning the value of chromatin as a mechanism to store/control information to be transcribed into RNA instead of as a processing system for genomic information. As described, the linear organization of the human genome intersects with the 3-D geometric elements observed on ChromSTEM imaging such that the positioning of elements emerges from physiochemical principles (FIG. 18)²⁻⁵. When paired with prior work showing that heterochromatin in human cells is functionally paired with transcriptional activation, the properties of domains are rooted in the physical genome. These include SA/V, diffusability, efficient packing, and the stabilization of intermediary complexes; these are all key processes in chemical reactions^{7, 8, 74} that are crucial to cell behavior (FIGS. 19-21). That these patterns are present both in protein-coding and non-protein coding genes indicates that regulation is rooted in the physiochemical properties of the transcriptional reactions and not in the end product function (FIG. 21). This encoding is manifested through the power-law geometric positioning between exons and volumetric segments, likely to coordinate appropriate positioning of these transcribed segments for efficient reactions to occur for the ideal configuration. Indeed, the organizational principle indicates that an exon is functionally paired with an adjacent volumetric segment as the structures remain stable when these are permuted together (FIGS. 22c-d). It is notable that while most of the human and mouse genomes organize by this principle (FIGS. 19, 20, 22, and 23), the transformation from exon-rich configurations to volumetric appears to correlate with the degree of body plan complexity. That this organization intersects with, but is not a reflection of, genome connectivity suggests that these are likely co-regulatory processes (FIG. 24) that warrants further investigation. Having proved the hypothesis that the lengths of exons, introns, and intergenic segments geometrically encode the SA/V principles of packing domains to interact with transcription, several new fundamental questions arise. The following transformative positions for evolution, aging/development, transcriptional regulation, and genomic function are proposed based on the intersection of the molecular and physical genome:

1) Domain geometry facilitates the rapid emergence of body plan diversity. A transformation is demonstrated from linear into power-law geometries from S. cerevisiae onward to humans (FIGS. 19-23). This transformation parallels increasing body plan complexity, potentially converging at the time of the Cambrian Expansion⁷⁵⁻⁷⁷. Are packing domains a non-mutational innovation that facilitated the rapid expansion by storing transcriptional states for prolonged periods (FIG. 23g)? From this physical perspective, modularity of domains allows a rapid, non-mutationally driven generation of new cell states, shapes, and behaviors. This occurs because changing configurations does not require any sequence mutations. The selective pressure converges instead on gene position, orientation, and segment lengths to generate cohesive assemblies. What is perceived as ‘neutral drift’ of the sequence is potentially under a different facet of fitness. This allows increased sampling of these regions for potentially beneficial states while maintaining a ‘default’ state as volumetric elements. These findings suggest that selection is driven by the benefit of the system, and not necessarily the gene alone^{11, 12}. Such a transformation would build upon prior cellular innovations (genes, nucleosomes, splicing, nuclear envelopes, mitochondria) to magnify the degrees of freedom in the genome. The patterns observed in the position of coding/volumetric elements appear to indicate the domain SA/V information that guides transcription reactions became crucial enough to propagate within the linear human and mouse genomes. Future work analyzing the phylogenetic transformation of metazoans around the Cambrian Expansion through this lens of geometry and physical genomics can address this question. Whether similar principles organize plant and archaeal genomes could further our understanding of genome organization translating into function.

### 2) SA/V Stabilization of Mature Domains Defines Cell Responses Across Decades.

Nuclear swelling and heterochromatin loss are hallmarks of aging across human tissues⁷⁸⁻⁸⁰. Tissue development is defined by the non-random deposition of heterochromatin⁸¹. While TADs, loops, and compartments appear resilient to nuclear volume, the SA/V ratios in packing domains would be profoundly impacted by these changes^{5, 82, 83}. Among the risk factors for Alzheimers' Disease is the read-through fusion of ApoE and Tomm4084 (FIG. 18a). Is nuclear expansion and heterochromatin loss contributing to transcriptional ‘fusions’ by positioning segments along the ideal zone surface as a single element⁹? Could similar processes be involved in epithelial disruption in inflammatory diseases⁸⁵? Do chromosome fusions, fragmentation, and copy number variations transform the positioning of exons to produce pathological states in cancers⁸⁶? Is it possible to control exon skipping by modifying the chain length or hinge element? Analysis of these transformations and their manipulation based on physical principles could provide insights to and treatments for these and other complex diseases. Likewise, even as all domains are unified assemblies of heterochromatin and euchromatin integrated into a single element, the radius of a domain and the efficiency of packing will impact the proportions that are surface relative to the volume generated. Could the interior of the nucleus be enriched primarily in relative smaller, efficiently packed domains resulting in an abundance of surface features (A-/euchromatin)? Conversely, could the periphery be dominated by larger core volumes leading to a perception of increased core features (B-/heterochromatin)? Could some features of connectivity, loops/TADs, represent the connectivity along packing domain surfaces (FIG. 24)? If so, this would explain why the transformation from low resolution imaging to nanoscopic studies results in a distinct transformation of the architecture^{2-4, 33, 87, 88}. When considered in the context that excluded volume impacts the capacity of relatively large antibodies to target high density domain cores, this provides a framework to unify observations across length-scales.

### 3) Transcriptional Memories Define Domain Complexity.

Among the mysteries of transcriptional patterns is excess binding sites for key transcription factors (e.g. Myod) that are not utilized by the mature tissue^{89, 90}. An elegant solution is that these elements are replicated in a manner like other non-coding regions in order to provide potential sources of new domains. The observation that SA/V organizes in the direction of the reading frame indicates that a coding segment in the anti-parallel direction can instead act as a volumetric element. Since segment length and not necessarily sequence needs to be conserved to give rise to SA/V properties, could the insertion of transcription factor binding sites allow the ability for future testing of possible domain states while integrating with existing machinery? In effect, the default positions needed for a human body plan are encoded in the measured positions of coding/volumetric pairs, but potential conformations can be realized to generate new adaptations. If these adaptations improve fitness, they could be hard coded for the next progeny. Repositioning of these elements and the deposition of domain-defining elements in the linear assembly could provide insight into how sequence specificity intersects with geometric specificity for regulatory processes.

4) Chromatin Generates ‘Geometric Computational Units’ to Integrate Current Signals with Past States

By considering physical properties of the genome at the intersection with transcription reactions, these elements mirror aspects of learning, computation, and neural networks^{91, 92}. This work demonstrates that the transcriptional memory of domains is imprinted onto the genome itself to provide heritable and durable complexity. By considering the physical genome through this lens, is it more appropriate to consider packing domains as ‘geometric computational domains’? Some of the positional elements observed mirror elements of logic operators or computations, such as “AND” where genes in a paired segment become co-transcribed and “OR” states where one gene competes with another for space. Further, transcriptional memories echo the process of reinforcement learning in neural networks.^{91, 92} Since the spacing includes introns and intergenic segments, it is a system to coherently optimize the physiochemical conditions in the nucleus. Viewed from the perspective of a processing system, increasing the complexity of the genome in 1-D (low M or high γ) is solved by reducing complexity in the ideal surface from 3D geometry adopted by the chain. The expansion of unused transcription factor binding sites or gene fragments that act as volumetric elements until engaged indicates that many additional configurations (latency) can be realized in human cells if needed. Acting as geometric processors, the structures formed would have a broad latitude to encode cell-specific plans by coordinating the timing and segmentation into SA/V units. If conditions were to change, alterations in configuration provide a rapid source of sampling information. That many primordial transcription factors (e.g. Sox2, Nanog) are information dense in the linear segments suggest they could be efficiently transcribed even as configurations were transformed throughout the nucleus. One could test this by permuting the order of signaling events and measuring if configurations are predictably paired to the transcriptional response. Finally, one could consider the synthesis of novel genes, gene therapies, or whole chromosomes in cells by manipulating the SA/V coupling between a coding-/volumetric-pairs.

This insight indicates that manipulation of non-transcribed non-coding elements to control the surface area to volume distribution of genomic contents is programmable. How changing the length and positioning between exons, genes, or sets of genes will impact complex transcriptional states can be predicted. This contrasts with the existing probabilistic approach to gene activation. Instead of combinations of the correct signals being the primary determinant of cell behavior, knowing what configurations are present, what configurations are possible, and how to change the positions will generate new behaviors to the exact same signals.

The observation that volumetric DNA emerged with increasing body plan complexity indicates that evolutionary fitness can be programmed by manipulating the surface area to volume principles of domains in different organisms. This creates a non-mutational toolbox for rapid genomic sampling to occur as configurations can be shifted so that new responses to stressors can be identified. Since this represents a new schema for evolutionary fitness by providing organisms with more advanced ‘geometric processing unit’ to generate novel solutions to new stressors.

Putting these concepts together, these findings create the principles necessary to generate coherent novel chromosomes with non-random orientation of gene contents to create new complex traits for multicellular interactions. These synthetic chromosomes in principle can be transferred into existing organisms to improve fitness (e.g. enhance coral response to pH, temperature and oxygen tension at all once with modules that are driven by the environment). This ability to generate geometrically encoded computations within complex genetic contents creates an avenue for the generation of de novo multicellular scaffolds for geometric computational systems. Similar computing attempts have been proposed with polymers, DNA sequence, and simple organisms, however, the information capacity within packing domain geometry is several orders of magnitude greater in scope. A single packing domain composed of 100 Kbp with the goal of generating ˜40 positional elements has 10\hat{ }33 possible states. The formed configurations respond to inputs including the total volume of the system, the concentrations of enzymes, the strength and recurrence of the input signal, the ionic conditions, and the redox state. This generates multiple input/output pathways for computation.

### REFERENCES

1. Ou, H. D. et al. ChromEMT: Visualizing 3D chromatin structure and compaction in interphase and mitotic cells. Science (1979) 357, (2017).
2. Li, Y. et al. Nanoscale chromatin imaging and analysis platform bridges 4D chromatin organization with molecular function. Sci Adv 7, (2021).
3. Li, Y. et al. Analysis of three-dimensional chromatin packing domains by chromatin scanning transmission electron microscopy (ChromSTEM). Sci Rep 12, 12198 (2022).
4. Wang, X. et al. Chromatin reprogramming and bone regeneration in vitro and in vivo via the microtopography-induced constriction of cell nuclei. Nat Biomed Eng 7, 1514-1529 (2023).
5. Carignano, M. A. et al. Local volume concentration, packing domains, and scaling properties of chromatin. Elife 13, (2024).
6. Almassalha, L. M. et al. Macrogenomic engineering via modulation of the scaling of chromatin packing density. Nat Biomed Eng 1, 902-913 (2017).
7. Matsuda, H., Putzel, G. G., Backman, V. & Szleifer, I. Macromolecular Crowding as a Regulator of Gene Transcription. Biophys J 106, 1801-1810 (2014).
8. Tan, C., Saurabh, S., Bruchez, M. P., Schwartz, R. & LeDuc, P. Molecular crowding shapes gene expression in synthetic cellular nanosystems. Nat Nanotechnol 8, 602-608 (2013).
9. Pabis, K. et al. A concerted increase in readthrough and intron retention drives transposon expression during aging and senescence. Elife 12, (2024).
10. Muniz, L. et al. Control of Gene Expression in Senescence through Transcriptional Read-Through of Convergent Protein-Coding Genes. Cell Rep 21, 2433-2446 (2017).
11. Doolittle, W. F. & Sapienza, C. Selfish genes, the phenotype paradigm and genome evolution. Nature 284, 601-603 (1980).
12. Orgel, L. E. & Crick, F. H. C. Selfish DNA: the ultimate parasite. Nature 284, 604-607 (1980).
13. Ohno, S. So much ‘junk’ DNA in our genome. Brookhaven Symp Biol. 23, 366-370 (1972).
14. Cavalier-Smith, T. Nuclear volume control by nucleoskeletal DNA, selection for cell volume and cell growth rate, and the solution of the DNA C-value paradox. J Cell Sci 34, 247-278 (1978).
15. Hong, J., Cavga, A. D., Shah, D., Laue, E. & Taipale, J. Scaling laws of human transcriptional activity. Preprint at doi.org/10.1101/2023.08.10.551625 (2023).
16. Hernández-Hernández, J. M., García-González, E. G., Brun, C. E. & Rudnicki, M. A. The myogenic regulatory factors, determinants of muscle development, cell identity and regeneration. Semin Cell Dev Biol 72, 10-18 (2017).
17. Gao, T. & Qian, J. EnhancerAtlas 2.0: an updated resource with enhancer annotation in 586 tissue/cell types across nine species. Nucleic Acids Res (2019) doi: 10.1093/nar/gkz980.
18. Brown, J. M. et al. A tissue-specific self-interacting chromatin domain forms independently of enhancer-promoter interactions. Nat Commun 9, 3849 (2018).
19. Hsieh, T.-H. S. et al. Enhancer-promoter interactions and transcription are largely maintained upon acute loss of CTCF, cohesin, WAPL or YY1. Nat Genet 54, 1919-1932 (2022).
20. Lieberman-Aiden, E. et al. Comprehensive Mapping of Long-Range Interactions Reveals Folding Principles of the Human Genome. Science (1979) 326, 289-293 (2009).
21. Rao, S. S. P. et al. A 3D Map of the Human Genome at Kilobase Resolution Reveals Principles of Chromatin Looping. Cell 159, 1665-1680 (2014).
22. Rao, S. S. P. et al. Cohesin Loss Eliminates All Loop Domains. Cell 171, 305-320.e24 (2017).
23. Bintu, B. et al. Super-resolution chromatin tracing reveals domains and cooperative interactions in single cells. Science (1979) 362, (2018).
24. Boettiger, A. N. et al. Super-resolution imaging reveals distinct chromatin folding for different epigenetic states. Nature 529, 418-422 (2016).
25. Hafner, A. et al. Loop stacking organizes genome folding from TADs to chromosomes. Mol Cell 83, 1377-1392.e6 (2023).
26. Miron, E. et al. Chromatin arranges in chains of mesoscale domains with nanoscale functional topography independent of cohesin. Sci Adv 6, (2020).
27. Szabo, Q. et al. Regulation of single-cell genome organization into TADs and chromatin nanodomains. Nat Genet 52, 1151-1157 (2020).
28. Szabo, Q., Bantignies, F. & Cavalli, G. Principles of genome folding into topologically associating domains. Sci Adv 5, (2019).
29. Otterstrom, J. et al. Super-resolution microscopy reveals how histone tail acetylation affects DNA compaction within nucleosomes in vivo. Nucleic Acids Res 47, 8470-8484 (2019).
30. Castells-Garcia, A. et al. Super resolution microscopy reveals how elongating RNA polymerase II and nascent RNA interact with nucleosome clutches. Nucleic Acids Res 50, 175-190 (2022).
31. Neguembor, M. V. et al. MiOS, an integrated imaging and computational strategy to model gene folding with nucleosome resolution. Nat Struct Mol Biol 29, 1011-1023 (2022).
32. Ricci, M. A., Manzo, C., García-Parajo, M. F., Lakadamyali, M. & Cosma, M. P. Chromatin Fibers Are Formed by Heterogeneous Groups of Nucleosomes In Vivo. Cell 160, 1145-1158 (2015).
33. Pujadas Liwag, E. M. et al. Depletion of lamins B1 and B2 promotes chromatin mobility and induces differential gene expression by a mesoscale-motion-dependent mechanism. Genome Biol 25, 77 (2024).
34. Maeshima, K. et al. The physical size of transcription factors is key to transcriptional regulation in chromatin domains. Journal of Physics: Condensed Matter 27, 064116 (2015).
35. Solovei, I. et al. Spatial Preservation of Nuclear Chromatin Architecture during Three-Dimensional Fluorescence in Situ Hybridization (3D-FISH). Exp Cell Res 276, 10-23 (2002).
36. Brown, J. M., De Ornellas, S., Parisi, E., Schermelleh, L. & Buckle, V. J. RASER-FISH: non-denaturing fluorescence in situ hybridization for preservation of three-dimensional interphase chromatin structure. Nat Protoc 17, 1306-1331 (2022).
37. Shim, A. R. et al. Formamide denaturation of double-stranded DNA for fluorescence in situ hybridization (FISH) distorts nanoscale chromatin structure. PLOS One 19, e0301000 (2024).
38. Virk, R. K. A. et al. Disordered chromatin packing regulates phenotypic plasticity. Sci Adv 6, (2020).
39. Statello, L., Guo, C.-J., Chen, L.-L. & Huarte, M. Gene regulation by long non-coding RNAs and its biological functions. Nat Rev Mol Cell Biol 22, 96-118 (2021).
40. Shang, R., Lee, S., Senavirathne, G. & Lai, E. C. microRNAs in action: biogenesis, function and regulation. Nat Rev Genet 24, 816-833 (2023).
41. Morris, K. V. & Mattick, J. S. The rise of regulatory RNA. Nat Rev Genet 15, 423-437 (2014).
42. Nozaki, T. et al. Condensed but liquid-like domain organization of active chromatin regions in living human cells. Sci Adv 9, (2023).
43. Talbert, P. B. & Henikoff, S. Histone variants at a glance. J Cell Sci 134, (2021).
44. Zhang, M. et al. Angle between DNA linker and nucleosome core particle regulates array compaction revealed by individual-particle cryo-electron tomography. Nat Commun 15, 4395 (2024).
45. Szerlong, H. J. & Hansen, J. C. Nucleosome distribution and linker DNA: connecting nuclear function to dynamic chromatin structure This paper is one of a selection of papers published in a Special Issue entitled 31st Annual International Asilomar Chromatin and Chromosomes Conference, and has undergone the Journal's usual peer review process. Biochemistry and Cell Biology 89, 24-34 (2011).
46. Ibarra, J. et al. Differentiation-dependent chromosomal organization changes in normal myogenic cells are absent in rhabdomyosarcoma cells. Front Cell Dev Biol 11, (2023).
47. Sabaté, T. et al. Universal dynamics of cohesin-mediated loop extrusion. bioRxiv 2024.08.09.605990 (2024) doi: 10.1101/2024.08.09.605990.
48. Gabriele, M. et al. Dynamics of CTCF- and cohesin-mediated chromatin looping revealed by live-cell imaging. Science (1979) 376, 496-501 (2022).
49. Brückner, D. B., Chen, H., Barinov, L., Zoller, B. & Gregor, T. Stochastic motion and transcriptional dynamics of pairs of distal DNA loci on a compacted chromosome. Science (1979) 380, 1357-1362 (2023).
50. Bancaud, A. et al. Molecular crowding affects diffusion and binding of nuclear proteins in heterochromatin and reveals the fractal organization of chromatin. EMBO J 28, 3785-3798 (2009).
51. Metze, K. Fractal dimension of chromatin: potential molecular diagnostic applications for cancer prognosis. Expert Rev Mol Diagn 13, 719-735 (2013).
52. Almassalha, L. M. et al. The Global Relationship between Chromatin Physical Topology, Fractal Structure, and Gene Expression. Sci Rep 7, 41061 (2017).
53. Mirny, L. A. The fractal globule as a model of chromatin architecture in the cell. Chromosome Research 19, 37-51 (2011).
54. Oesterreich, F. C. et al. Splicing of Nascent RNA Coincides with Intron Exit from RNA Polymerase II. Cell 165, 372-381 (2016).
55. Shenasa, H. & Bentley, D. L. Pre-mRNA splicing and its cotranscriptional connections. Trends Genet 39, 672-685 (2023).
56. Zeng, Y. et al. Profiling lariat intermediates reveals genetic determinants of early and late co-transcriptional splicing. Mol Cell 82, 4681-4699.e8 (2022).
57. O'Leary, N. A. et al. Reference sequence (RefSeq) database at NCBI: current status, taxonomic expansion, and functional annotation. Nucleic Acids Res 44, D733-D745 (2016).
58. Lonsdale, J. et al. The Genotype-Tissue Expression (GTEx) project. Nat Genet 45, 580-585 (2013).
59. Takahashi, K. & Yamanaka, S. A decade of transcription factor-mediated reprogramming to pluripotency. Nat Rev Mol Cell Biol 17, 183-193 (2016).
60. Pearson, J. C., Lemons, D. & McGinnis, W. Modulating Hox gene functions during animal body patterning. Nat Rev Genet 6, 893-904 (2005).
61. Goding, C. R. & Arnheiter, H. MITF—the first 25 years. Genes Dev 33, 983-1007 (2019).
62. Kim, W.-J., Shin, H.-L., Kim, B.-S., Kim, H.-J. & Ryoo, H.-M. RUNX2-modifying enzymes: therapeutic targets for bone diseases. Exp Mol Med 52, 1178-1184 (2020).
63. Gerhard, G. S. Comparative aspects of zebrafish (Danio rerio) as a model for aging research. Exp Gerontol 38, 1333-1341 (2003).
64. Fatica, A. & Bozzoni, I. Long non-coding RNAs: new players in cell differentiation and development. Nat Rev Genet 15, 7-21 (2014).
65. Derrien, T. et al. The GENCODE v7 catalog of human long noncoding RNAs: Analysis of their gene structure, evolution, and expression. Genome Res 22, 1775-1789 (2012).
66. Liao, J. et al. Long noncoding RNA (lncRNA) H19: An essential developmental regulator with expanding roles in cancer, stem cell differentiation, and metabolic diseases. Genes Dis 10, 1351-1366 (2023).
67. Neguembor, M. V. et al. Transcription-mediated supercoiling regulates genome folding and loop formation. Mol Cell 81, 3065-3081.e12 (2021).
68. Janissen, R., Barth, R., Polinder, M., van der Torre, J. & Dekker, C. Single-molecule visualization of twin-supercoiled domains generated during transcription. Nucleic Acids Res 52, 1677-1687 (2024).
69. Delamarre, A. et al. Chromatin architecture mapping by multiplex proximity tagging. Preprint at doi.org/10.1101/2024.11.12.623258 (2024).
70. Fullwood, M. J. et al. An oestrogen-receptor-α-bound human chromatin interactome. Nature 462, 58-64 (2009).
71. Hitz, B. C. et al. The ENCODE Uniform Analysis Pipelines “Elements of Order 4404 E Oregon St, Bellingham WA 98226, USA. doi: 10.1101/2023.04.04.535623.
72. An integrated encyclopedia of DNA elements in the human genome. Nature 489, 57-74 (2012).
73. Luo, Y. et al. New developments on the Encyclopedia of DNA Elements (ENCODE) data portal. Nucleic Acids Res 48, D882-D889 (2020).
74. Putzel, G. G., Tagliazucchi, M. & Szleifer, I. Nonmonotonic Diffusion of Particles Among Larger Attractive Crowding Spheres. Phys Rev Lett 113, 138302 (2014).
75. Zhang, X. & Shu, D. Current understanding on the Cambrian Explosion: questions and answers. PalZ 95, 641-660 (2021).
76. Roy, M., Kim, N., Xing, Y. & Lee, C. The effect of intron length on exon creation ratios during the evolution of mammalian genomes. RNA 14, 2261-2273 (2008).
77. Ruiz-Trillo, I., Kin, K. & Casacuberta, E. The Origin of Metazoan Multicellularity: A Potential Microbial Black Swan Event. Annu Rev Microbiol 77, 499-516 (2023).
78. Lee, J.-H., Kim, E. W., Croteau, D. L. & Bohr, V. A. Heterochromatin: an epigenetic point of view in aging. Exp Mol Med 52, 1466-1474 (2020).
79. Kedlian, V. R. et al. Human skeletal muscle aging atlas. Nat Aging (2024) doi: 10.1038/s43587-024-00613-3.
80. Pathak, R. U., Soujanya, M. & Mishra, R. K. Deterioration of nuclear morphology and architecture: A hallmark of senescence and aging. Ageing Res Rev 67, 101264 (2021).
81. Nicetto, D. & Zaret, K. S. Role of H3K9me3 heterochromatin in cell identity establishment and maintenance. Curr Opin Genet Dev 55, 1-10 (2019).
82. Liu, Y. & Dekker, J. CTCF-CTCF loops and intra-TAD interactions show differential dependence on cohesin ring integrity. Nat Cell Biol 24, 1516-1527 (2022).
83. Sanders, J. T. et al. Loops, topologically associating domains, compartments, and territories are elastic and robust to dramatic nuclear volume swelling. Sci Rep 12, 4721 (2022).
84. Xu, J. et al. TOMM40-APOE chimera linking Alzheimer's highest risk genes: a new pathway for mitochondria regulation and APOE4 pathogenesis. Preprint at doi.org/10.1101/2024.10.09.617477 (2024).
85. Stankey, C. T. et al. A disease-associated gene desert directs macrophage inflammation through ETS2. Nature 630, 447-456 (2024).
86. Bailey, C. et al. Origins and impact of extrachromosomal DNA. Nature 635, 193-200 (2024).
87. Falk, M. et al. Heterochromatin drives compartmentalization of inverted and conventional nuclei. Nature 570, 395-399 (2019).
88. Rahman, F. et al. Mapping the nuclear landscape with multiplexed super-resolution fluorescence microscopy. Preprint at doi.org/10.1101/2024.07.27.605159 (2024).
89. Yang, Z. et al. MyoD and E-protein heterodimers switch rhabdomyosarcoma cells from an arrested myoblast phase to a differentiated state. Genes Dev 23, 694-707 (2009).
90. Cao, Y. et al. Genome-wide MyoD Binding in Skeletal Muscle Cells: A Potential for Broad Cellular Reprogramming. Dev Cell 18, 662-674 (2010).
91. Kaelbling, L. P., Littman, M. L. & Moore, A. W. Reinforcement Learning: A Survey. Journal of Artificial Intelligence Research 4, 237-285 (1996).
92. van Otterlo, M. & Wiering, M. Reinforcement Learning and Markov Decision Processes. in 3-42 (2012). doi: 10.1007/978-3-642-27645-3_1.
93. Shim, A. R. et al. Dynamic Crowding Regulates Transcription. Biophys J 118, 2117-2129 (2020).
94. Stirling, D. R. et al. CellProfiler 4: improvements in speed, utility and usability. BMC Bioinformatics 22, 433 (2021).
95. Schindelin, J. et al. Fiji: an open-source platform for biological-image analysis. Nat Methods 9, 676-682 (2012).

We claim:

1. A method of regulating gene transcription in a cell comprising:
forming one or more chromatin packing domain in the cell; stabilizing the one or more chromatin packing domain in the cell; and preventing decay of the stabilized chromatin packing domain.

2. The method of claim 1, wherein forming the one or more chromatin packing domain comprises forming a nascent chromatin packing domain.

3. The method of claim 1, wherein the nascent chromatin packing domain is formed by one or more of transient contact of chromatin fiber from spatial confinement, loop extrusion, and transcriptionally mediated contact of chromatin fiber.

4. The method of claim 1, wherein stabilizing the one or more chromatin packing domain comprises maintaining or increasing nucleosome-modifying enzymes and ion concentration in the domain.

5. The method of claim 4, wherein the nucleosome-modifying enzymes are selected from heterochromatin enzyme and euchromatin enzyme.

6. The method of claim 4, wherein the ion is calcium and/or magnesium.

7. The method of claim 1, wherein the stabilized chromatin packing domain comprises a high-density inner core, an intermediate zone, and low-density outer zone.

8. The method of claim 7, wherein the high-density inner core comprises heterochromatin enzyme.

9. The method of claim 7, wherein the intermediate zone comprises heterochromatin enzyme and/or RNA polymerase enzyme.

10. The method of claim 7, wherein low-density outer zone comprises euchromatin enzyme.

11. The method of claim 1, wherein forming one or more chromatin packing domain comprises modulating a surface-to-volume assembly of chromatin segments.

12. The method of claim 11, wherein modulating a surface-to-volume assembly comprises pairing exons with an adjacent volumetric DNA segments.

13. The method of claim 12, wherein the volumetric DNA segments comprise introns and intergenic segments.
